SEQUENCE LISTING (29 sequences)

SEQ ID NO: 1 - DNA, 1104 bp, Artificial Sequence
  Factor VIII A2 subunit nucleic acid sequence
gatcctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaagatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaac

SEQ ID NO: 2 - protein, 368 aa, Artificial Sequence
  Factor VIII A2 subunit amino acid sequence
SVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPR

SEQ ID NO: 3 - DNA, 483 bp, Artificial Sequence
  Factor VIII C2 subunit nucleic acid sequence
gatccaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacc

SEQ ID NO: 4 - protein, 161 aa, Artificial Sequence
  Factor VIII C2 subunit amino acid sequence
NSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 5 - DNA, 135 bp, Artificial Sequence
  CD8 alpha chain hinge
ctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcct

SEQ ID NO: 6 - DNA, 75 bp, Artificial Sequence
  Transmembrane domain
ccggaatctacatctgggcccctctggccggcacctgtggcgtgctgctgctgtccctggtcatcaccctgtact

SEQ ID NO: 7 - protein, 45 aa, Artificial Sequence
  CD8 alpha chain hinge
TTTPAPRPPTPAPTIASQPLSLRPEACRPAAGGAVHTRGLDFACD

SEQ ID NO: 8 - protein, 25 aa, Artificial Sequence
  Transmembrane domain
IYIWAPLAGTCGVLLLSLVITLYCK

SEQ ID NO: 9 - DNA, 123 bp, Artificial Sequence
  Intracellular signaling domain of 4-1BB
gcaagcggggcagaaagaagctgctgtacatcttcaagcagcccttcatgcggcctgtgcagaccacacaggaagaggacggctgtagctgtagattccccgaggaagaggaaggcggctgcg

SEQ ID NO: 10 - protein, 40 aa, Artificial Sequence
  4-1BB intracellular signaling domain
GRKKLLYIFKQPFMRPVQTTQEEDGCSCRFPEEEEGGCEL

SEQ ID NO: 11 - DNA, 336 bp, Artificial Sequence
  CD3 zeta signaling domain
agctgagagtgaagttcagcagaagcgccgacgcccctgcctatcagcagggccagaaccagctgtacaacgagctgaacctgggcagacgggaggaatacgacgtgctggacaagagaagaggccgggaccctgagatgggcggcaagcccagacggaagaacccccaggaaggcctgtataacgaactgcagaaagacaagatggccgaggcctacagcgagatcggcatgaagggcgagcggagaagaggcaagggccatgacggcctgtaccagggcctgagcaccgccaccaaggacacctacgacgccctgcacatgcaggccctgcctc

SEQ ID NO: 12 - protein, 111 aa, Artificial Sequence
  CD3 zeta signaling domain
VKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - DNA, 10335 bp, Artificial Sequence
  pELPS-hFVIII-A2-BBz-T2A-mCherry
gatctatggagtttgggctgagctggctttttcttgtggctattttaaaaggtgtccagtgcggatcctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaagatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagagctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgattccggaatctacatctgggcccctctggccggcacctgtggcgtgctgctgctgtccctggtcatcaccctgtactgcaagcggggcagaaagaagctgctgtacatcttcaagcagcccttcatgcggcctgtgcagaccacacaggaagaggacggctgtagctgtagattccccgaggaagaggaaggcggctgcgagctgagagtgaagttcagcagaagcgccgacgcccctgcctatcagcagggccagaaccagctgtacaacgagctgaacctgggcagacgggaggaatacgacgtgctggacaagagaagaggccgggaccctgagatgggcggcaagcccagacggaagaacccccaggaaggcctgtataacgaactgcagaaagacaagatggccgaggcctacagcgagatcggcatgaagggcgagcggagaagaggcaagggccatgacggcctgtaccagggcctgagcaccgccaccaaggacacctacgacgccctgcacatgcaggccctgcctccaagaggcagcggagagggcagaggaagtcttctaacatgcggtgacgtggaggagaatcccggccctacgcgtatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctgcattgatcacgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagag

SEQ ID NO: 14 - protein, 875 aa, Artificial Sequence
  hFVIII-A2-BBz-T2A-mCherry
MEFGLSWLFLVAILKGVQCGSSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRASTTTPAPRPPTPAPTIASQPLSLRPEACRPAAGGAVHTRGLDFACDSGIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPTRMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 15 - protein, 616 aa, Artificial Sequence
  hFVIII-A2-BBz-T2A
MEFGLSWLFLVAILKGVQCGSSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRASTTTPAPRPPTPAPTIASQPLSLRPEACRPAAGGAVHTRGLDFACDSGIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - DNA, 9714 bp, Artificial Sequence
  pELPS-hFVIII-C2-BBz-T2A-mCherry
gatctatggagtttgggctgagctggctttttcttgtggctattttaaaaggtgtccagtgcggatccaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctacgctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgattccggaatctacatctgggcccctctggccggcacctgtggcgtgctgctgctgtccctggtcatcaccctgtactgcaagcggggcagaaagaagctgctgtacatcttcaagcagcccttcatgcggcctgtgcagaccacacaggaagaggacggctgtagctgtagattccccgaggaagaggaaggcggctgcgagctgagagtgaagttcagcagaagcgccgacgcccctgcctatcagcagggccagaaccagctgtacaacgagctgaacctgggcagacgggaggaatacgacgtgctggacaagagaagaggccgggaccctgagatgggcggcaagcccagacggaagaacccccaggaaggcctgtataacgaactgcagaaagacaagatggccgaggcctacagcgagatcggcatgaagggcgagcggagaagaggcaagggccatgacggcctgtaccagggcctgagcaccgccaccaaggacacctacgacgccctgcacatgcaggccctgcctccaagaggcagcggagagggcagaggaagtcttctaacatgcggtgacgtggaggagaatcccggccctacgcgtatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctgcattgatcacgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagag

SEQ ID NO: 17 - protein, 668 aa, Artificial Sequence
  pELPS-hFVIII-C2-BBz-T2A-mCherry
MEFGLSWLFLVAILKGVQCGSNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYASTTTPAPRPPTPAPTIASQPLSLRPEACRPAAGGAVHTRGLDFACDSGIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPTRMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 18 - protein, 409 aa, Artificial Sequence
  hFVIII-C2-BBz
MEFGLSWLFLVAILKGVQCGSNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYASTTTPAPRPPTPAPTIASQPLSLRPEACRPAAGGAVHTRGLDFACDSGIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - DNA, 9547 bp, Artificial Sequence
  pTRPE-hFVIII-A2-BBz
gtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctgcatacgcgtcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagagccaccatggagtttgggctgagctggctttttcttgtggctattttaaaaggtgtccagtgcggatcctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaagatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagagctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgattccggaatctacatctgggcccctctggccggcacctgtggcgtgctgctgctgtccctggtcatcaccctgtactgcaagcggggcagaaagaagctgctgtacatcttcaagcagcccttcatgcggcctgtgcagaccacacaggaagaggacggctgtagctgtagattccccgaggaagaggaaggcggctgcgagctgagagtgaagttcagcagaagcgccgacgcccctgcctatcagcagggccagaaccagctgtacaacgagctgaacctgggcagacgggaggaatacgacgtgctggacaagagaagaggccgggaccctgagatgggcggcaagcccagacggaagaacccccaggaaggcctgtataacgaactgcagaaagacaagatggccgaggcctacagcgagatcggcatgaagggcgagcggagaagaggcaagggccatgacggcctgtaccagggcctgagcaccgccaccaaggacacctacgacgccctgcacatgcaggccctgcctccaagatgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgg

SEQ ID NO: 20 - DNA, 8926 bp, Artificial Sequence
  pTRPE-hFVIII-C2-BBz
gtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctgcatacgcgtcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagagccaccatggagtttgggctgagctggctttttcttgtggctattttaaaaggtgtccagtgcggatccaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctacgctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgattccggaatctacatctgggcccctctggccggcacctgtggcgtgctgctgctgtccctggtcatcaccctgtactgcaagcggggcagaaagaagctgctgtacatcttcaagcagcccttcatgcggcctgtgcagaccacacaggaagaggacggctgtagctgtagattccccgaggaagaggaaggcggctgcgagctgagagtgaagttcagcagaagcgccgacgcccctgcctatcagcagggccagaaccagctgtacaacgagctgaacctgggcagacgggaggaatacgacgtgctggacaagagaagaggccgggaccctgagatgggcggcaagcccagacggaagaacccccaggaaggcctgtataacgaactgcagaaagacaagatggccgaggcctacagcgagatcggcatgaagggcgagcggagaagaggcaagggccatgacggcctgtaccagggcctgagcaccgccaccaaggacacctacgacgccctgcacatgcaggccctgcctccaagatgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgg

SEQ ID NO: 21 - DNA, 1848 bp, Artificial Sequence
  DAP12-T2A-A2-KIRS2
atggggggacttgaaccctgcagcaggttcctgctcctgcctctcctgctggctgtaagtggtctccgtcctgtccaggtccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaagtcgagggcggcggagagggcagaggaagtcttctaacatgcggtgacgtggaggagaatcccggccctaggatggccttaccagtgaccgccttgctcctgccgctggccttgctgctccacgccgccaggccgggatcctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaagatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagagctagcggtggcggaggttctggaggtgggggttcctcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcataa

SEQ ID NO: 22 - protein, 478 aa, Artificial Sequence
  FVIII-A2-KIRS2
MALPVTALLLPLALLLHAARPGSSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRASGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 23 - DNA, 1227 bp, Artificial Sequence
  DAP12-T2A-C2-KIRS2
atggggggacttgaaccctgcagcaggttcctgctcctgcctctcctgctggctgtaagtggtctccgtcctgtccaggtccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaagtcgagggcggcggagagggcagaggaagtcttctaacatgcggtgacgtggaggagaatcccggccctaggatggccttaccagtgaccgccttgctcctgccgctggccttgctgctccacgccgccaggccgggatccaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctacgctagcggtggcggaggttctggaggtgggggttcctcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcataa

SEQ ID NO: 24 - protein, 271 aa, Artificial Sequence
  FVIII-C2-KIRS2
MALPVTALLLPLALLLHAARPGSNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYASGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 25 - DNA, 1746 bp, Artificial Sequence
  A2-gs-BBz Nucleotide Sequence
atggagtttgggctgagctggctttttcttgtggctattttaaaaggtgtccagtgcggatcctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaagatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagagctagcggtggcggaggttctggaggtggaggttcctccggaatctacatctgggcccctctggccggcacctgtggcgtgctgctgctgtccctggtcatcaccctgtactgcaagcggggcagaaagaagctgctgtacatcttcaagcagcccttcatgcggcctgtgcagaccacacaggaagaggacggctgtagctgtagattccccgaggaagaggaaggcggctgcgagctgagagtgaagttcagcagaagcgccgacgcccctgcctatcagcagggccagaaccagctgtacaacgagctgaacctgggcagacgggaggaatacgacgtgctggacaagagaagaggccgggaccctgagatgggcggcaagcccagacggaagaacccccaggaaggcctgtataacgaactgcagaaagacaagatggccgaggcctacagcgagatcggcatgaagggcgagcggagaagaggcaagggccatgacggcctgtaccagggcctgagcaccgccaccaaggacacctacgacgccctgcacatgcaggccctgcctccaagatga

SEQ ID NO: 26 - protein, 581 aa, Artificial Sequence
  A2-gs-BBz Amino Acid Sequence
MEFGLSWLFLVAILKGVQCGSSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRASGGGGSGGGGSSGIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - DNA, 1125 bp, Artificial Sequence
  C2-gs-BBz Nucleic Acid Sequence
atggagtttgggctgagctggctttttcttgtggctattttaaaaggtgtccagtgcggatccaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctacgctagcggtggcggaggttctggaggtggaggttcctccggaatctacatctgggcccctctggccggcacctgtggcgtgctgctgctgtccctggtcatcaccctgtactgcaagcggggcagaaagaagctgctgtacatcttcaagcagcccttcatgcggcctgtgcagaccacacaggaagaggacggctgtagctgtagattccccgaggaagaggaaggcggctgcgagctgagagtgaagttcagcagaagcgccgacgcccctgcctatcagcagggccagaaccagctgtacaacgagctgaacctgggcagacgggaggaatacgacgtgctggacaagagaagaggccgggaccctgagatgggcggcaagcccagacggaagaacccccaggaaggcctgtataacgaactgcagaaagacaagatggccgaggcctacagcgagatcggcatgaagggcgagcggagaagaggcaagggccatgacggcctgtaccagggcctgagcaccgccaccaaggacacctacgacgccctgcacatgcaggccctgcctccaagatga

SEQ ID NO: 28 - protein, 374 aa, Artificial Sequence
  C2-gs-BBz Amino Acid Sequence
MEFGLSWLFLVAILKGVQCGSNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYASGGGGSGGGGSSGIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 5 aa, Artificial Sequence
  Glycine-serine linker
GGGGS

(19) United States
(12) Patent Application Publication
(10) Pub. No.: US 2022/0220188 A1
(43) Pub. Date: Jul. 14, 2022

(54) COMPOSITIONS AND METHODS OF CHIMERIC ALLOANTIGEN RECEPTOR T CELLS

(71) Applicants: THE TRUSTEES OF THE UNIVERSITY OF PENNSYLVANIA, Philadelphia, PA (US); THE CHILDREN'S HOSPITAL OF PHILADELPHIA, Philadelphia, PA (US)

(72) Inventors: Michael C. MILONE, Cherry Hill, NJ (US); Valder ARRUDA, Philadelphia, PA (US); Sarah RICHMAN, Philadelphia, PA (US); Benjamin SAMELSON-JONES, Philadelphia, PA (US)

(21) Appl. No.: 17/409,354

(22) Filed: Aug. 23, 2021

Related U.S. Application Data
(63) Continuation of application No. 16/093,539, filed on Oct. 12, 2018, now abandoned.
(60) Provisional application No. 62/322,937, filed on Apr. 15, 2016.

(51) Int. Cl. C07K 14/705; A61K 35/17; C07K 14/00; C12N 5/0783; A61P 7/04; C07K 14/725; C07K 14/755

(52) U.S. Cl. CPC C07K 14/70532; A61K 35/17; C07K 14/00; C12N 5/0638; C07K 19/00; C07K 14/70503; C07K 14/7051; C07K 14/70517; C07K 14/755; A61P 7/04

(57) ABSTRACT

The invention includes compositions comprising at least one chimeric alloantigen receptor (CALLAR) specific for an alloantibody, vectors comprising the same, compositions comprising CALLAR vectors packaged in viral particles, and recombinant T cells comprising the CALLAR. The invention also includes methods of making a genetically modified T cell expressing a CALLAR, wherein the expressed CALLAR comprises a Factor VIII or fragment thereof extracellular domain.

### CROSS-REFERENCE TO RELATED APPLICATION

This application is a continuation of U.S. patent application Ser. No. 16/093,539, filed Oct. 12, 2018, which is a 35 U.S.C. § 371 national phase application from, and claims priority to, International Application No. PCT/US2017/027754, filed Apr. 14, 2017, and published under PCT Article 21(2) in English, which claims priority to U.S. Provisional Application Ser. No. 62/322,937, filed Apr. 15, 2016, the contents of all of which are incorporated by reference herein in their entireties.

### STATEMENT REGARDING SEQUENCE LISTING SUBMITTED ELECTRONICALLY VIA EFS-WEB

The content of the electronically submitted sequence listing in ASCII text file (Name: 046483-7105US2_Sequence_Listing; Size: 108,238 bytes; and Date of Creation: Aug. 23, 2021) is herein incorporated by reference in its entirety.

### BACKGROUND OF THE INVENTION

Hemophilia A is an inherited X-linked disease caused by Factor VIII (FVIII) deficiency and is a serious and life-threatening bleeding disorder. In addition to a ˜1% per year risk of death due to intracranial hemorrhage, hemophila A is associated with frequent hemarthosis and arthropathy that causes significant morbidity for patients. Factor replacement therapy using recombinant human FVIII (rhFVIII) is the standard of care for patients with hemophilia A. Unfortunately, 10-40% of patients with hemophilia develop antibodies to plasma-derived or recombinant human FVIII protein concentrate that inhibit FVIII function. At low titer, the presence of these inhibitory antibodies necessitates increased FVIII to overcome their effects resulting in markedly increased costs of therapy. At high titer, these inhibitory antibodies can render factor replacement therapy useless placing patients at significantly increased risk of hemarthrosis and catastrophic intracranial bleeding requiring the use of by-pass agents.

Currently, there are no FDA-approved therapies for the elimination of FVIII inhibitors. Immune interventions including cyclophosphamide, IVIg, Rituximab (anti-CD20) and plasmapharesis have been evaluated to reduce the level of these inhibitory FVIII antibodies along with attempts to eliminate them by immune tolerance induction. While there has been success in a limited number of patients, these approaches generally lead to only transient reductions in inhibitory antibody titers.

Novel strategies are therefore needed to effectively diminish the inhibitory antibodies that represent a major barrier to successful FVIII replacement therapy.

### SUMMARY OF THE INVENTION

The invention includes an isolated nucleic acid sequence encoding a chimeric alloantigen receptor (CALLAR), wherein the isolated nucleic acid sequence comprises a nucleic acid sequence encoding an alloantigen or fragment thereof, a nucleic acid sequence encoding a transmembrane domain, a nucleic acid sequence encoding an intracellular signaling domain of 4-1BB, and a nucleic acid sequence encoding a CD3 zeta signaling domain.

Further included is an isolated nucleic acid sequence encoding a chimeric alloantigen receptor (CALLAR), wherein the isolated nucleic acid sequence comprises a nucleic acid sequence encoding an A2 subunit of Factor VIII, a nucleic acid sequence v a transmembrane domain, a nucleic acid sequence v an intracellular domain of a costimulatory molecule, and a nucleic acid sequence encoding an intracellular signaling domain.

In some embodiments, the alloantigen is Factor VIII or fragment thereof and the Factor VIII fragment thereof is selected from the group consisting of an A2 subunit or a C2 subunit of Factor VIII. In other embodiments, the Factor VIII or fragment thereof comprises an amino acid sequence selected from the group consisting of SEQ ID NO:2 and SEQ ID NO:4. In yet additional embodiments, wherein the nucleic acid sequence of the transmembrane domain encodes a CD8 alpha chain hinge and transmembrane domain. In further embodiments, he CD8 alpha chain hinge comprises an amino acid sequence of SEQ ID NO:7 and transmembrane domain comprises an amino acid sequence of SEQ ID NO:8. In yet other embodiments, the nucleic acid sequence encoding the intracellular domain of the costimulatory molecule comprises a nucleic acid sequence encoding a 4-1BB signaling domain. In further embodiments, the 4-1BB intracellular domain comprises an amino acid sequence of SEQ ID NO:10. In yet other embodiments, the nucleic acid sequence encoding the intracellular signaling domain comprises a nucleic acid sequence encoding a CD3 zeta signaling domain. In additional embodiments, the CD3 zeta signaling domain comprises an amino acid sequence of SEQ ID NO:12.

The invention additionally includes a vector comprising the isolated nucleic acid sequence the invention, wherein, in certain embodiments, the vector is an RNA vector, for example, a lentiviral vector.

Also included is an isolated chimeric alloantigen receptor (CALLAR) comprising an extracellular domain comprising an alloantigen or fragment thereof, a transmembrane domain, an intracellular domain of 4-1BB, and a CD3 zeta signaling domain.

In one aspect, there is provided an isolated chimeric alloantigen receptor (CALLAR) comprising an extracellular domain comprising A2 subunit of Factor VIII, a transmembrane domain, an intracellular domain of a costimulatory molecule, and an intracellular signaling domain.

Also included is a genetically modified cell comprising the CALLAR of the invention. In some embodiments, the cell expresses the CALLAR and has high affinity to antibodies expressed on B cells. In other embodiments, the cell expresses the CALLAR and induces killing of B cells expressing antibodies. In additional embodiments, the cell expresses the CALLAR and has limited toxicity toward healthy cells. In other embodiments, the cell is selected from the group consisting of a helper T cell, a cytotoxic T cell, a memory T cell, regulatory T cell, gamma delta T cell, a natural killer cell, a monocyte, a cytokine induced killer cell, a cell line thereof, and other effector cell.

The invention also includes a method for treating a disorder associated with FVIII antibodies in a subject with hemophilia, the method comprising: administering to the subject an effective amount of a genetically modified T cell comprising an isolated nucleic acid sequence encoding a chimeric alloantigen receptor (CALLAR), wherein the isolated nucleic acid sequence comprises a nucleic acid sequence encoding an alloantigen or fragment thereof, a nucleic acid sequence encoding a transmembrane domain, a nucleic acid sequence encoding an intracellular signaling domain of 4-1BB, and a nucleic acid sequence encoding a CD3 zeta signaling domain, thereby treating the disorder associated with FVIII antibodies in the subject with hemophilia.

Additionally, the invention includes a method for treating a disorder associated with FVIII antibodies in a subject with hemophilia, the method comprising: administering to the subject an effective amount of a genetically modified T cell comprising an isolated nucleic acid sequence encoding a chimeric alloantigen receptor (CALLAR), wherein the isolated nucleic acid sequence comprises a nucleic acid sequence encoding A2 subunit of Factor VIII, a nucleic acid sequence encoding a transmembrane domain, a nucleic acid sequence encoding an intracellular domain of a costimulatory molecule, and a nucleic acid sequence encoding an intracellular signaling domain, thereby treating the disorder associated with FVIII antibodies in the subject with hemophilia.

In some embodiments, the subject is a human. In other embodiments, the modified T cell has high affinity for Factor VIII antibodies. In other embodiments, the modified T cell targets a B cell expressing Factor VIII antibodies.

Also included in the invention is an isolated KIR/DAP12 receptor complex comprising a chimeric alloantigen receptor (CALLAR) comprising an A2 subunit of Factor VIII or C2 subunit of Factor VIII; a linker; and a fragment of a KIR comprising a transmembrane region and a cytoplasmic domain, and DAP12.

In some embodiments, the KIR is KIRS2 or KIR2DS2. In other embodiments, the linker is a short glycine-serine linker.

Also included is a genetically modified cell comprising an isolated KIR/DAP12 receptor complex.

Further included is a genetically modified cell comprising: an isolated chimeric alloantigen receptor (CALLAR) and DAP12, wherein the CALLAR comprises an extracellular domain comprising A2 subunit of Factor VIII or C2 subunit of Factor VIII, a linker, and a fragment of a KIR, wherein the KIR comprises a transmembrane region and a cytoplasmic domain. In some embodiments, the KIR is KIRS2 or KIR2DS2. In other embodiments, the linker is a short glycine-serine linker.

Also included is a method for treating a disorder associated with FVIII antibodies in a subject with hemophilia. The method comprises administering to the subject an effective amount of a genetically modified T cell comprising: an isolated nucleic acid sequence encoding a chimeric alloantigen receptor (CALLAR) comprising a nucleic acid sequence encoding A2 subunit of Factor VIII or C2 subunit of Factor VIII; a nucleic acid sequence encoding a linker; a nucleic acid sequence encoding a fragment of a KIR comprising a transmembrane region and a cytoplasmic domain, and further comprising a nucleic sequence encoding DAP12, thereby treating the disorder associated with FVIII antibodies in the subject with hemophilia.

In some embodiments, the linker is a short glycine-serine linker.

Further included is a method for treating a disorder associated with FVIII antibodies in a subject with hemophilia. The method comprises administering to the subject an effective amount of a genetically modified T cell comprising a chimeric alloantigen receptor (CALLAR) comprising an A2 subunit of Factor VIII or C2 subunit of Factor VIII, a linker, a fragment of a KIR comprising a transmembrane region and a cytoplasmic domain, and further comprising DAP12, thereby treating the disorder associated with FVIII antibodies in the subject with hemophilia.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is an illustration of FVIII chimeric alloantigen receptor (CALLAR).

FIG. 2 is an illustration of exemplary CALLAR constructs bearing alternate signaling domains or extracellular hinges as compared to FIG. 1.

The design on the left side of the figure represents an illustration of a chimeric alloantigen receptor (CALLAR comprising an A2 or C2 subunit of Factor VIII, a transmembrane domain (CD8), an intracellular signaling domain of 4-1BB, and a CD3 zeta signaling domain.

The design in the center of the figure represents an illustration of a chimeric alloantigen receptor (CALLAR) comprising an A2 or C2 subunit of Factor VIII, a linker (short glycine-serine linker (gs)), a transmembrane domain (CD8), an intracellular signaling domain of 4-1BB, and a CD3 zeta signaling domain.

The design on the right side of the figure represents an illustration of a KIR2DS2-based chimeric immunoreceptor in which the A2 or C2 domain of Factor VIII (FVIII) is fused to the transmembrane and cytoplasmic domains of KIRS2 with a short glycine-serine linker between the FVIII domain and the KIR sequence. This chimeric receptor is expressed with the DAP12 adaptor protein to produce a chimeric KIR/DAP12 receptor complex.

FIG. 3 is a panel of graphs illustrating surface expression of A2 and C2 CALLAR on human T cells. T cells were activated with CD3/28 beads for 24 hrs followed by lentiviral transduction of an A2-CALLAR or C2-CALLAR utilizing the 4-1BB and Zeta signaling domains (A2bbz and C2bbz, respectively). Lentiviral vectors expressing A2- or C2-CALLAR constructs (A2bbz-mCh or C2bbz-mCh) were also generated and used for transduction. FMC63bbz CAR (anti-CD19 CAR) was used as a control. T cells were stained with either an A2 or C2 specific antibodies as indicated on day 5 following transduction to detect expression of the A2 and C2 containing CALLARs. Protein L was used to stain for the FMC63bbz CAR.

Flow cytometry was used to analyze A2 and C2-based CARs on primary T-cells. Fresh isolated human T cells from healthy donors were transduced with lentiviral vector supernatants encoding the following CARs: FMC63-bbz, A2-bbz, and C2-bbz. A2bbz-mCh and C2bbz-mCh represent T cells transduced with lentiviral vectors encoding a bi-cistronic construct for expression of the respective CAR and mCherry as separate proteins. CAR expression was evaluated by flow cytometry. Briefly, T cells were cultured in RPMI 1640 medium with 10% FBS and stimulated with anti-CD3/anti-CD28 Dynabeads (invitrogen). 24 hrs after stimulation, T cells were transduced with the CAR lentiviral vector supernatants. 6-8 days after lentiviral transduction T cells were stained with biotinylated Protein L antibody followed by strepavidin PE (BD Biosciences), anti-A2 followed by or goat-anti mouse-FITC (Jackson ImmunoResearch), or anti-C2 followed by or goat-anti mouse-FITC (Jackson ImmunoResearch) as indicated. CAR expression was evaluated by flow cytometry (LSR-II, BD). Flow cytometry analysis was carried out by using Flowjo (Tree Star Inc). After transduction it was observed that A2 and C2 domain-based CARs were efficiently expressed on the cell surface of the transduced T cells.

FIG. 4 is a graph illustrating activation of A2 and C2 CALLAR-modified T cells by immobilized anti-A2 or anti-C2 antibodies. T cells transduced with indicated CAR or CALLAR were plated on microwells coated with OKT3 (for polyclonal T cell activation), anti-A2 or anti-C2. Supernatants were harvest at 24 hours, and IFN-γ was measured by ELISA. Results illustrate that all T cells are capable of producing IFNγ following activation by anti-CD3 antibody. Only A2-BBz transduced T cells produce IFNγ in response to A2-specific antibody. Only C2-BBz transduced T cells produce IFNγ in response to C2-specific antibody.

FIG. 5 is a graph illustrating a CALLAR model system for antigen-specific B cells. CD19+ Nalm6 cells were engineered to express FVIII-specific chimeric immunoglobulin. Human peripheral blood T cells were transduced with A2-FVIII-CALLARs (A2-CALLARs), C2-FVIII-CALLARs (C2-CALLARs), Dsg3-CAAR or CD19-CAR (controls) or non-transduced T cells (NTD). The T cells were mixed with Nalm6 cells engineered to express surface immunoglobulin specific for the A2 domain of FVIII at varying effector to target (E:T) ratios. Percent specific lysis was measured by a 51Cr release assay at 16 hours.

FIG. 6 is a set of graphs illustrating antibody-specific cytotoxicity using an A2-domain containing or a C2-domain containing chimeric alloantibody receptor (CALLAR) with a CD8 extracellular spacer. T cells were transduced with lentiviral vectors encoding an anti-CD19 CAR (19BBz), an A2-domain containing chimeric alloantibody receptor with a CD8 extracellular spacer (A2(cd8)BBz) or a C2-domain containing receptor with the same CD8 spacer (C2(cd8)BBz). 19BBz-expressing T cells only show cytotoxicity towards the CD19+ target K562 cells. A2(cd8)BBz transduced T cells only mediate lysis of K562 target cells expressing anti-A2 surface immunoglobulin. C2(cd8)BBz transduced T cells only mediate lysis of K562 target cells expressing anti-C2 surface immunoglobulin.

FIG. 7 is a set of graphs illustrating antibody-specific cytotoxicity using an A2-domain containing or a C2-domain containing chimeric alloantibody receptor with (Gly)₄-Ser extracellular spacer or linker. T cells were transduced with lentiviral vectors encoding an anti-CD19 CAR (19BBz), an A2-domain containing chimeric alloantibody receptor with a synthetic (Gly)₄-Ser extracellular spacer (A2(gs)BBz) or a C2-domain containing receptor with the same (Gly)₄-Ser spacer (C2(gs)BBz). 19BBz-expressing T cells only show cytotoxicity towards the CD19+ target K562 cells. A2(gs)BBz transduced T cells only mediate lysis of K562 target cells expressing anti-A2 surface immunoglobulin. C2(gs)BBz transduced T cells only mediate lysis of K562 target cells expressing anti-C2 surface immunoglobulin.

FIG. 8 is a set of graphs illustrating antibody-specific cytotoxicity using an A2-domain containing or a C2-domain containing chimeric alloantibody receptor with KIR/DAP12-based signaling. T cells were transduced with lentiviral vectors encoding an anti-CD19 CAR (19BBz), an A2-domain containing chimeric alloantibody receptor with KIR/DAP12 signaling (A2(gs)KIRS2) or a C2-domain containing receptor with the same KIR/DAP12 signaling (C2(gs)KIRS2). 19BBz-expressing T cells only show cytotoxicity towards the CD19+ target K562 cells. A2(gs)KIRS2-transduced T cells only mediate lysis of K562 target cells expressing anti-A2 surface immunoglobulin. C2(gs)KIRS2-transduced T cells only mediate lysis of K562 target cells expressing anti-C2 surface immunoglobulin.

FIG. 9 is a set of graphs illustrating cytokine production in response to antibody on the cell surface. T cells were transduced with lentiviral vectors encoding an anti-CD19 CAR (19BBz), A2-domain containing chimeric alloantibody receptors with a CD8 extracellular spacer (A2(cd8)BBz), a synthetic (Gly)₄-Ser (A2(gs)BBz) or with KIR/DAP12 signaling (A2(gs)KIRS2), or C2-domain containing receptor with the same CD8 spacer (C2(cd8)BBz), synthetic (Gly)₄-Ser (C2(gs)BBz) or with KIR/DAP12 signaling (C2(gs)KIRS2). 19BBz-expressing T cells only show enhanced IFNγ production in response to CD19+ target K562 cells or CD3/28 beads. A2(cd8)BBz, A2(gs)BBz and A2(gs)KIRS2 T cells show enhanced IFNγ production in response to K562 target cells expressing anti-A2 surface immunoglobulin or positive control CD3/28 beads. C2(cd8)BBz, C2(gs)BBz and C2(gs)KIRS2 T cells show enhanced IFNγ production in response to K562 target cells expressing anti-C2 surface immunoglobulin or positive control CD3/28 beads.

### DETAILED DESCRIPTION

The invention includes compositions and methods of using a chimeric alloantigen receptor (CALLAR) specific for an alloantibody, wherein the expressed CALLAR comprises a Factor VIII or fragment thereof in the extracellular domain.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice of and/or for the testing of the present invention, the preferred materials and methods are described herein. In describing and claiming the present invention, the following terminology will be used according to how it is defined, where a definition is provided.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, in some instances ±5%, in some instances ±1%, and in some instance ±0.1% from the specified value, as such variations are appropriate to perform the disclosed methods.

The term “antibody,” as used herein, refers to an immunoglobulin molecule binds with an antigen. Antibodies can be intact immunoglobulins derived from natural sources or from recombinant sources and can be immunoreactive portions of intact immunoglobulins. Antibodies are typically tetramers of immunoglobulin molecules. The antibody in the present invention may exist in a variety of forms where the antibody is expressed as part of a contiguous polypeptide chain including, for example, a single domain antibody fragment (sdAb), a single chain antibody (scFv) and a humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “high affinity” as used herein refers to high specificity in binding or interacting or attraction of one molecule to a target molecule.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

By “alloantigen” is meant an antigen present only in some individuals (such as a particular blood group) of a species and capable of inducing the production of an alloantibody by individuals that lack the alloantigen.

The term “limited toxicity” as used herein, refers to the peptides, polynucleotides, cells and/or antibodies of the invention manifesting a lack of substantially negative biological effects, anti-tumor effects, or substantially negative physiological symptoms toward a healthy cell, non-tumor cell, non-diseased cell, non-target cell or population of such cells either in vitro or in vivo.

“Alloantibody” refers to an antibody that is produced by a B cell specific for an alloantigen.

As used herein, the term “autologous” is meant to refer to any material derived from the same individual to which it is later to be re-introduced into the individual.

“Allogeneic” refers to a graft derived from a different animal of the same species.

“Xenogeneic” refers to a graft derived from an animal of a different species.

“Chimeric alloantigen receptor” or “CALLAR” refers to an engineered receptor that is expressed on a T cell or any other effector cell type capable of cell-mediated cytotoxicity. The CALLAR includes an alloantigen or fragment thereof that is specific for an alloantibody. The CALLAR also includes a transmembrane domain, a costimulatory domain and a signaling domain.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, for example, one or more amino acid residues within the extracellular regions of the CALLAR of the invention can be replaced with other amino acid residues having a similar side chain or charge and the altered CALLAR can be tested for the ability to bind autoantibodies using the functional assays described herein.

“Co-stimulatory ligand,” as the term is used herein, includes a molecule on an antigen presenting cell (e.g., an aAPC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Effective amount” or “therapeutically effective amount” are used interchangeably herein, and refer to an amount of a compound, formulation, material, or composition, as described herein effective to achieve a particular biological result. Such results may include, but are not limited to, the inhibition of virus infection as determined by any means suitable in the art.

The term “effector function” refers to a specialized function of a cell.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by a promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes), retrotransposons (e.g. piggyback, sleeping beauty), and viruses (e.g., lentiviruses, retroviruses, adenoviruses, and adeno-associated viruses) that incorporate the recombinant polynucleotide.

The term “Factor VIII” refers to a blood-clotting protein, also known as anti-hemophilic factor. Factor VIII is encoded by the F8 gene in humans and produces two alternatively spliced transcripts. Factor VIII is a cofactor of Factor IXa, which forms a complex that converts Factor X to the activated form, Xa. Factor VIII is a non-covalent heterodimer comprised of a heavy chain (A1-A2-B subunits) and light chain (A3-C1-C2 subunits) that circulates as an inactive procofactor in a complex with von Willebrand factor.

The term “Factor VIII antibody” refers to an antibody that specifically binds to FVIII blood-clotting protein. The FVIII antibody includes alloantibodies and autoantibodies that are specific for FVIII.

The term “hemophilia” refers to a blood clotting disorder. Hemophilia A refers to a recessive, X-linked genetic disorder in individuals that lack functional Factor VIII. Hemophilia B refers to a recessive, X-linked genetic disorder in individuals that lack functional Factor IX.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The phrase “an immunologically effective amount,” “an anti-alloantibody effective amount,” or “therapeutic amount” as used herein refers to the amount of the composition of the present invention to be administered, determined by a researcher or physician with consideration of individual differences in age, weight, tumor size, extent of infection or metastasis, and condition of the patient (subject).

The term “intracellular signaling domain” refers to the portion of a protein which transduces the effector function signal and directs the cell to perform a specialized function. The intracellular signaling domain includes any truncated portion of the intracellular domain sufficient to transduce the effector function signal.

As used herein, an “instructional material” includes a publication, a recording, a diagram, or any other medium of expression that can be used to communicate the usefulness of the compositions and methods of the invention. The instructional material of the kit of the invention may, for example, be affixed to a container that contains the nucleic acid, peptide, and/or composition of the invention or be shipped together with a container that contains the nucleic acid, peptide, and/or composition. Alternatively, the instructional material may be shipped separately from the container with the intention that the instructional material and the compound be used cooperatively by the recipient.

“Intracellular domain” refers to a portion or region of a molecule that resides inside a cell.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “proinflammatory cytokine” refers to a cytokine or factor that promotes inflammation or inflammatory responses. Examples of proinflammatory cytokines include, but are not limited to, chemokines (CCL, CXCL, CX3CL, XCL), interleukins (such as, IL-1, IL-2, IL-3, IL-5, IL-6, IL-7, IL-9, IL10 and IL-15), interferons (IFNγ), and tumor necrosis factors (TNFα and TNFβ).

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence that is required for expression of a gene product operably linked to the promoter/regulatory sequence. In some instances, this sequence may be the core promoter sequence and in other instances, this sequence may also include an enhancer sequence and other regulatory elements that are required for expression of the gene product. The promoter/regulatory sequence may, for example, be one that expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

A “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

“Signaling domain” refers to the portion or region of a molecule that recruits and interacts with specific proteins in response to an activating signal.

By the term “specifically binds,” as used herein, is meant an antibody, or a ligand, which recognizes and binds with a cognate binding partner (e.g., a stimulatory and/or costimulatory molecule present on a T cell) protein present in a sample, but which antibody or ligand does not substantially recognize or bind other molecules in the sample.

The term “subject” is intended to include living organisms in which an immune response can be elicited (e.g., mammals).

As used herein, a “substantially purified” cell is a cell that is essentially free of other cell types. A substantially purified cell also refers to a cell that has been separated from other cell types with which it is normally associated in its naturally occurring state. In some instances, a population of substantially purified cells refers to a homogenous population of cells. In other instances, this term refers simply to cells that have been separated from the cells with which they are naturally associated in their natural state. In some embodiments, the cells are cultured in vitro. In other embodiments, the cells are not cultured in vitro.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one that has been transfected, transformed or transduced with exogenous nucleic acid. The cell includes the primary subject cell and its progeny.

“Transmembrane domain” refers to a portion or a region of a molecule that spans a lipid bilayer membrane.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-β, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an WIC Class I molecule loaded with a peptide, an anti-CD3 antibody, a superagonist anti-CD28 antibody, and a superagonist anti-CD2 antibody.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

A method for eliminating FVIII-specific B cells while leaving normal B-cell immunity intact is the most desirable therapeutic approach to treat hemophilia, because chronic, non-specific immunosuppression using anti-CD20 antibody and other non-specific immunosuppressive modalities are associated with increased risk of serious infection. Chimeric antigen receptor (CAR) technology has been successfully developed for the treatment of B-cell malignancies. While a B-cell specific CAR (such as a CD19 CAR) might be beneficial in eliminating memory B cells that produce Factor VIII (FVIII) antibodies, B cells destined to secrete anti-FVIII alloantibodies express surface anti-FVIII antibody. Targeting this unique and highly restricted marker on these alloantigen-specific B cells provides a therapeutic opportunity to eliminate the B cells producing FVIII-specific antibodies that interfere with FVIII therapy.

### Chimeric AlloAntigen Receptor (CALLAR)

The present invention is based in part on the discovery that chimeric alloantigen receptors can be used to target alloantibodies produced in response to FVIII replacement treatment. Alloantibodies are produced in some individuals who receive recombinant or purified FVIII as treatment for their FVIII deficiency. Individuals with hemophilia have a genetic deficiency of FVIII. Since they do not have FVIII due to genetic abnormalities that disrupt the FVIII gene, FVIII appears foreign to their immune system and their cells make antibodies against FVIII. The invention includes compositions comprising a CALLAR specific for an alloantibody, vectors comprising the same, compositions comprising CALLAR vectors packaged in viral particles, and recombinant T cells or other effector cells comprising the CALLAR. The invention also includes methods of making a genetically modified T cell expressing a CALLAR, wherein the expressed CALLAR comprises a factor VIII or fragment thereof in the extracellular domain.

The antigens for many alloantibody-mediated diseases, such as FVIII replacement treatment in hemophilia, have been described. The present invention includes a technology for treating alloantibody-mediated diseases. In particular, technologies that target B cells that ultimately produce the auto- and alloantibodies and display the auto- and alloantibodies on their cell surfaces, mark these B cells as disease-specific targets for therapeutic intervention. The invention therefore includes a method for efficiently targeting and killing the pathogenic B cells by using an auto- and alloantibody-specific (e.g., Factor VIII) chimeric alloantigen receptor (or CALLAR). In one embodiment of the present invention, only specific anti-autoantibody- and anti-alloantibody-expressing B cells are killed, thus leaving intact the beneficial B cells and antibodies that protect from infection.

The present invention encompasses a recombinant DNA construct comprising nucleic acid sequences that encode an extracellular domain comprising an alloantigen or a fragment thereof, in one aspect, a human Factor VIII or fragment thereof, wherein the sequence of the alloantigen or fragment thereof is operably linked to a nucleic acid sequence encoding an intracellular signaling domain.

In one aspect, the invention includes an isolated nucleic acid sequence encoding a chimeric alloantigen receptor (CALLAR), wherein the isolated nucleic acid sequence comprises a nucleic acid sequence encoding an alloantigen or fragment thereof, a nucleic acid sequence encoding a transmembrane domain, a nucleic acid sequence encoding an intracellular signaling domain of 4-1BB, and a nucleic acid sequence encoding a CD3 zeta signaling domain.

In another aspect, the invention includes an isolated nucleic acid sequence encoding a chimeric alloantigen receptor (CALLAR), wherein the isolated nucleic acid sequence comprises a nucleic acid sequence encoding A2 subunit of Factor VIII, a nucleic acid sequence encoding a transmembrane domain, a nucleic acid sequence encoding an intracellular domain of a costimulatory molecule, and a nucleic acid sequence encoding an intracellular signaling domain.

In yet another aspect, the invention includes an isolated chimeric alloantigen receptor (CALLAR) comprising an extracellular domain comprising an alloantigen or fragment thereof, a transmembrane domain, an intracellular domain of 4-1BB, and a CD3 zeta signaling domain. In still another aspect, the invention includes an isolated chimeric alloantigen receptor (CALLAR) comprising an extracellular domain comprising A2 subunit of Factor VIII, a transmembrane domain, an intracellular domain of a costimulatory molecule, and an intracellular signaling domain.

Alloantigen Moiety

In one aspect, the constructs described herein comprise a genetically engineered chimeric alloantigen receptor (CALLAR) comprising an extracellular domain comprising an alloantigen or fragment thereof. In one embodiment, the alloantigen is a Factor VIII or a fragment thereof. In an exemplary embodiment, the CALLAR comprises a Factor VIII A2 or C2 subunit. In another embodiment, the CALLAR comprises a Factor VIII subunit selected from the group consisting of an A1, an A2, an A3, a B, a C1, and a C2 subunit.

In one embodiment, the isolated nucleic acid sequence encoding the CALLAR comprises a nucleic acid sequence encoding a Factor VIII A2 subunit, comprising

GATCCTCAGTTGCCAAGAAGCATCCTAAAACTTGGGTACATTACATTGCT
GCTGAAGAGGAGGACTGGGACTATGCTCCCTTAGTCCTCGCCCCCGATGA
CAGAAGTTATAAAAGTCAATATTTGAACAATGGCCCTCAGCGGATTGGTA
GGAAGTACAAAAAAGTCCGATTTATGGCATACACAGATGAAACCTTTAAG
ACTCGTGAAGCTATTCAGCATGAATCAGGAATCTTGGGACCTTTACTTTA
TGGGGAAGTTGGAGACACACTGTTGATTATATTTAAGAATCAAGCAAGCA
GACCATATAACATCTACCCTCACGGAATCACTGATGTCCGTCCTTTGTAT
TCAAGGAGATTACCAAAAGGTGTAAAACATTTGAAGGATTTTCCAATTCT
GCCAGGAGAAATATTCAAATATAAATGGACAGTGACTGTAGAAGATGGGC
CAACTAAATCAGATCCTCGGTGCCTGACCCGCTATTACTCTAGTTTCGTT
AATATGGAGAGAGATCTAGCTTCAGGACTCATTGGCCCTCTCCTCATCTG
CTACAAAGAATCTGTAGATCAAAGAGGAAACCAGATAATGTCAGACAAGA
GGAATGTCATCCTGTTTTCTGTATTTGATGAGAACCGAAGCTGGTACCTC
ACAGAGAATATACAACGCTTTCTCCCCAATCCAGCTGGAGTGCAGCTTGA
AGATCCAGAGTTCCAAGCCTCCAACATCATGCACAGCATCAATGGCTATG
TTTTTGATAGTTTGCAGTTGTCAGTTTGTTTGCATGAGGTGGCATACTGG
TACATTCTAAGCATTGGAGCACAGACTGACTTCCTTTCTGTCTTCTTCTC
TGGATATACCTTCAAACACAAAATGGTCTATGAAGACACACTCACCCTAT
TCCCATTCTCAGGAGAAACTGTCTTCATGTCGATGGAAAACCCAGGTCTA
TGGATTCTGGGGTGCCACAACTCAGACTTTCGGAACAGAGGCATGACCGC
CTTACTGAAGGTTTCTAGTTGTGACAAGAACACTGGTGATTATTACGAGG
ACAGTTAT GAAGATATT TCAGCATACT TGCTGAGTAA
AAACAATGCC ATTGAAC or SEQ ID NO: 1.

In another embodiment, the Factor VIII A2 subunit comprises amino acid sequence comprising

SVAKKHPKTWVHYIAAEEEDWDYAPLVLAPDDRSYKSQYLNNGPQRIGRK
YKKVRFMAYTDETFKTREAIQHESGILGPLLYGEVGDTLLIIFKNQASRP
YNIYPHGITDVRPLYSRRLPKGVKHLKDFPILPGEIFKYKWTVTVEDGPT
KSDPRCLTRYYSSFVNMERDLASGLIGPLLICYKESVDQRGNQIMSDKRN
VILFSVFDENRSWYLTENTQRFLPNPAGVQLEDPEFQASNIMHSINGYVF
DSLQLSVCLHEVAYWYILSIGAQTDFLSVFFSGYTFKHKMVYEDTLTLFP
FSGETVFMSMENPGLWILGCHNSDFRNRGMTALLKVSSCDKNTGDYYEDS
YEDISAYLLSKNNAIEPR or SEQ ID NO: 2.

In another embodiment, the isolated nucleic acid sequence encoding the CALLAR comprises a nucleic acid sequence encoding a Factor VIII C2 subunit comprising

GATCCAATAGTTGCAGCATGCCATTGGGAATGGAGAGTAAAGCAATATCA
GATGCACAGATTACTGCTTCATCCTACTTTACCAATATGTTTGCCACCTG
GTCTCCTTCAAAAGCTCGACTTCACCTCCAAGGGAGGAGTAATGCCTGGA
GACCTCAGGTGAATAATCCAAAAGAGTGGCTGCAAGTGGACTTCCAGAAG
ACAATGAAAGTCACAGGAGTAACTACTCAGGGAGTAAAATCTCTGCTTAC
CAGCATGTATGTGAAGGAGTTCCTCATCTCCAGCAGTCAAGATGGCCATC
AGTGGACTCTCTTTTTTCAGAATGGCAAAGTAAAGGTTTTTCAGGGAAAT
CAAGACTCCTTCACACCTGTGGTGAACTCTCTAGACCCACCGTTACTGAC
TCGCTACCTTCGAATTCACCCCCAGAGTTGGGTGCACCAGATTGCCCTGA
GGATGGAGGTTCTGGGCTGCGAGGCACAGGACC or SEQ ID NO: 3.

In another embodiment, the Factor VIII C2 subunit comprises amino acid sequence

NSCSMPLGMESKAISDAQITASSYFTNMFATWSPSKARLHLQGRSNAWRP
QVNNPKEWLQVDFQKTMKVTGVTTQGVKSLLTSMYVKEFLISSSQDGHQW
TLFFQNGKVKVFQGNQDSFTPVVNSLDPPLLTRYLRIHPQSWVHQIALRM
EVLGCEAQDLY or SEQ ID NO: 4.

In yet another embodiment, the isolated nucleic acid sequence encoding the CALLAR comprises a nucleic acid sequence with at least 80%, 85%, 90%, 91%, 92%, 93%, 94%, 95%, 96%, 97%, 98%, or 99% identity or homology to any nucleic acid sequence described herein. In another embodiment, the CALLAR comprises an amino acid sequence with at least 80%, 85%, 90%, 91%, 92%, 93%, 94%, 95%, 96%, 97%, 98%, or 99% identity or homology to any amino acid sequence described herein.

In a further embodiment, the CALLAR of the invention comprises an alloantibody binding domain otherwise referred to as an alloantigen or a fragment thereof. The choice of alloantigen for use in the present invention depends upon the type of antibody being targeted. For example, the alloantigen may be chosen because it recognizes an antibody on a target cell, such as a B cell, associated with a particular disease state, e.g. FVIII replacement therapy in hemophilia.

In some instances, it is beneficial that the alloantibody binding domain is derived from the same species in which the CALLAR will ultimately be used. For example, for use in humans, it may be beneficial that the alloantibody binding domain of the CALLAR comprises an alloantigen that binds the alloantibody or a fragment thereof. Thus, in one embodiment, the alloantibody binding domain portion comprises an epitope of the alloantigen that binds the alloantibody. The epitope is the part of the alloantigen that is specifically recognized by the alloantibody.

Linker

In some embodiments, the CALLAR comprises a short glycine-serine linker (gs). In some embodiments, the short glycine-serine linker is an extracellular linker. The short glycine-serine linker can have 0-20 repeats, for example, 1 repeat, 2 repeats, etc., with each repeat having a length of 2-20 amino acids. In some embodiments, a single short glycine-serine linker repeat has a sequence of, e.g., Gly-Gly-Gly-Gly-Ser (SEQ ID NO: 29). Other combinations of glycine and serine repeats may be used for the glycine-serine linker.

Transmembrane Domain

In one embodiment, the CALLAR comprises a transmembrane domain. In some embodiments, the transmembrane domain comprises a hinge and a transmembrane domain, such as, but not limited to, a human T cell surface glycoprotein CD8 alpha chain hinge and transmembrane domain. The human CD8 chain hinge and transmembrane domain provides cell surface presentation of the chimeric alloantigen receptor.

With respect to the transmembrane domain, in various embodiments, the CALLAR comprises a transmembrane domain that is fused to the extracellular domain of the CALLAR. In one embodiment, the CALLAR comprises a transmembrane domain that naturally is associated with one of the domains in the CALLAR. In some instances, the transmembrane domain is selected or modified by amino acid substitution to avoid binding to the transmembrane domains of the same or different surface membrane proteins in order to minimize interactions with other members of the receptor complex.

The transmembrane domain may be derived either from a natural or from a synthetic source. When the source is natural, the domain may be derived from any membrane-bound or transmembrane protein. In one embodiment, the transmembrane domain may be synthetic, in which case it will comprise predominantly hydrophobic residues such as leucine and valine. In one aspect a triplet of phenylalanine, tryptophan and valine will be found at each end of a synthetic transmembrane domain. Optionally, a short oligo- or polypeptide linker, between 2 and 10 amino acids in length may form the linkage between the transmembrane domain and the cytoplasmic signaling domain of the CALLAR. A glycine-serine doublet provides a particularly suitable linker.

In some instances, a variety of human hinges can be employed as well including the human Ig (immunoglobulin) hinge.

Examples of the hinge and/or transmembrane domain include, but are not limited to, a hinge and/or transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD154, KIR, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

A killer immunoglobulin-like receptor (KIR) includes all KIRs, e.g., KIR2 and KIR2DS2, a stimulatory killer immunoglobulin-like receptor.

In one embodiment, the nucleic acid sequence of the transmembrane domain encodes a CD8 alpha chain hinge comprising CTAGCACCACGACGCCAGCGCCGCGACCACCAACACCGGCGCCCACCATC GCGTCGCAGCCCCTGTCCCTGCGCCCAGAGGCGTGCCGGCCAGCGGCGGG GGGCGCAGTGCACACGAGGGGGCTGGACTTCGCCT or SEQ ID NO:5 and transmembrane domain comprising

CCGGAATCTACATCTGGGCCCCTCTGGCCGGCACCTGTGGCGTGCTGCTG
CTGTCCCTGGTCATCACCCTGTACT or SEQ ID NO: 6.

In another embodiment, the nucleic acid sequence of the transmembrane domain encodes a CD8 alpha chain hinge comprising TTTPAPRPPTPAPTIASQPLSLRPEACRPAAGGAVHTRGLDFACD or SEQ ID NO:7. and a transmembrane domain comprising

IYIWAPLAGTCGVLLLSLVITLYCK or SEQ ID NO: 8.

In yet another embodiment, the transmembrane domain comprises a CD8 alpha chain hinge and/or transmembrane domain.

Cytoplasmic Domain

The intracellular signaling domain or otherwise the cytoplasmic domain comprises, a costimulatory signaling domain and an intracellular signaling domain. The costimulatory signaling domain refers to a portion of the CALLAR comprising the intracellular signaling domain of a costimulatory molecule, such as 4-1BB. Costimulatory molecules include cell surface molecules that are required for an efficient T cell activation. The cytoplasmic domain or otherwise the intracellular signaling domain of the CALLAR of the invention, is responsible for activation of at least one of the normal effector functions of the immune cell in which the CALLAR has been placed in. The intracellular signaling domain refers to a portion of the CALLAR comprising the intracellular signaling domain, such as intracellular signaling domain of CD3 zeta.

Effector function of a T cell, for example, may be cytolytic activity or helper activity including the secretion of cytokines. While the entire intracellular signaling domain can be employed, in many cases it is not necessary to use the entire domain. To the extent that a truncated portion of the intracellular signaling domain is used, such truncated portion may be used in place of the intact domain as long as it transduces the effector function signal.

Examples of intracellular signaling domains for use in the CALLAR of the invention include, but are not limited to, the cytoplasmic portion of the T cell receptor (TCR) and co-receptors that act in concert to initiate signal transduction following antigen receptor engagement, as well as any derivative or variant of these elements and any synthetic sequence that has the same functional capability.

It is well recognized that signals generated through the TCR alone are insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory manner or in an inhibitory manner. Primary cytoplasmic signaling sequences that act in a stimulatory manner may contain signaling motifs which are known as immunoreceptor tyrosine-based activation motifs or ITAMs.

Examples of the intracellular signaling domain includes a fragment or domain from one or more molecules or receptors including, but are not limited to, CD3 zeta, CD3 gamma, CD3 delta, CD3 epsilon, CD86, common FcR gamma, FcR beta (Fc Epsilon Rib), CD79a, CD79b, Fcgamma RIIa, DAP10, DAP12 (an immunotyrosine-based activation motifs (ITAM)-containing adaptor), T cell receptor (TCR), CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, any KIR, e.g., KIR2, KIR2DS2, other co-stimulatory molecules described herein, any derivative, variant, or fragment thereof, any synthetic sequence of a co-stimulatory molecule that has the same functional capability, and any combination thereof.

In one embodiment, the intracellular signaling domain of the CALLAR comprises the CD3 zeta signaling domain by itself or in combination with one or more desired cytoplasmic domain(s) useful in the context of the CALLAR of the invention. For example, the intracellular signaling domain of the CALLAR can comprise a CD3 zeta chain portion and a costimulatory signaling domain of 4-1BB. The costimulatory signaling domain refers to a portion of the CALLAR comprising the intracellular signaling domain of a costimulatory molecule. A costimulatory molecule is a cell surface molecule other than an antigen receptor or its ligands that is required for an efficient response of lymphocytes to an antigen.

In another embodiment, the nucleic acid sequence of the intracellular signaling domain of a costimulatory molecule comprises a nucleic acid sequence encoding an intracellular signaling domain of 4-1BB comprising GCAAGCGGGGCAGAAAGAAGCTGCTGTACATCTTCAAGCAGCCCTTCATG CGGCCTGTGCAGACCACACAGGAAGAGGACGGCTGTAGCTGTAGATTCCC CGAGGAAGAGGAAGGCGGCTGCG or SEQ ID NO:9. In another embodiment, the nucleic acid sequence of the 4-1BB intracellular signaling domain encodes an amino acid sequence comprising

GRKKLLYIFKQPFMRPVQTTQEEDGCSCRFPEEEEGGCEL or
SEQ ID NO: 10.

In another embodiment, the nucleic acid sequence of the signaling domain comprises a nucleic acid sequence encoding a CD3 zeta signaling domain comprising AGCTGAGAGTGAAGTTCAGCAGAAGCGCCGACGCCCCTGCCTATCAGCAG GGCCAGAACCAGCTGTACAACGAGCTGAACCTGGGCAGACGGGAGGAAT ACGACGTGCTGGACAAGAGAAGAGGCCGGGACCCTGAGATGGGCGGCAA GCCCAGACGGAAGAACCCCCAGGAAGGCCTGTATAACGAACTGCAGAAA GACAAGATGGCCGAGGCCTACAGCGAGATCGGCATGAAGGGCGAGCGGA GAAGAGGCAAGGGCCATGACGGCCTGTACCAGGGCCTGAGCACCGCCAC CAAGGACACCTACGACGCCCTGCACATGCAGGCCCTGCCTC or SEQ ID NO:11. In another embodiment, the nucleic acid sequence of the CD3 zeta signaling domain encodes an amino acid sequence comprising

VKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTY
DALHMQALPPR or SEQ ID NO: 12.

In some embodiments, an isolated KIR/DAP12 receptor complex comprises an isolated nucleic acid sequence encoding a chimeric alloantigen receptor (CALLAR). The isolated nucleic acid sequence comprises a nucleic acid sequence encoding A2 subunit of Factor VIII or C2 subunit of Factor VIII; a nucleic acid sequence encoding a linker; a nucleic acid sequence encoding a transmembrane domain of a KIR, wherein the KIR contains a transmembrane region and a cytoplasmic domain and DAP12. Signaling is derived from the chimeric KIR (KIR-CAR or KIR-CALLAR) assembling with DAP12 to produce a functional receptor complex. In some embodiments, the KIR is KIRS2 or KIR2DS2.

In some embodiments, the invention includes a genetically modified cell comprising an isolated chimeric alloantigen receptor (CALLAR) and DAP12, wherein the CALLAR comprises an extracellular domain comprising A2 subunit of Factor VIII or C2 subunit of Factor VIII, a linker, and a fragment of a KIR, wherein the KIR contains a transmembrane region and a cytoplasmic domain.

In some embodiments, a method is provided for treating a disorder associated with FVIII antibodies in a subject with hemophilia. The method comprises administering to the subject an effective amount of a genetically modified T cell comprising: an isolated nucleic acid sequence encoding a chimeric alloantigen receptor (CALLAR), wherein the isolated nucleic acid sequence comprises a nucleic acid sequence encoding A2 subunit of Factor VIII or C2 subunit of Factor VIII; a nucleic acid sequence encoding a linker; a nucleic acid sequence encoding a transmembrane domain of a KIR; a nucleic acid sequence encoding a fragment of a KIR, wherein the KIR contains a transmembrane region and a cytoplasmic domain; and a nucleic acid sequence encoding DAP12, thereby treating the disorder associated with FVIII antibodies in the subject with hemophilia.

In some embodiments, the KIR of the isolated KIR/DAP12 receptor complex is KIRS2 or KIR2DS2. In some embodiments, the linker is a short glycine-serine linker. In some embodiments, the linker of the isolated KIR/DAP12 receptor complex is a short glycine-serine linker.

In some embodiments, the KIR/DAP12 receptor complex comprises one or more of the sequences of SEQ ID NOs: 21-24.

Other Domains

The CALLAR and the nucleic acid encoding the CALLAR may further comprise a signal peptide, such as a human CD8 alpha chain signal peptide. The human CD8 alpha signal peptide is responsible for the translocation of the receptor to the T cell surface. In one embodiment, the isolated nucleic acid sequence encoding the CALLAR comprises a nucleic acid sequence encoding a CD8 alpha chain signal peptide. In another embodiment, the CALLAR comprises a CD8 alpha chain signal peptide.

The CALLAR may also comprise a peptide linker. In one embodiment, the isolated nucleic acid sequence encoding the CALLAR comprises a nucleic acid sequence encoding a peptide linker between the nucleic acid sequence encoding the extracellular domains and the transmembrane domain.

In another embodiment, the intracellular domains of the CALLAR can be linked to each other in a random or specified order. Optionally, a short oligo- or polypeptide linker, for example, between 2 and 10 amino acids in length may form a linkage between the domains. A glycine-serine doublet is a particularly suitable linker.

Any domains and/or fragments of the CALLAR, vector, and the promoter may be amplified by PCR or any other means known in the art.

### Vector Comprising the CALLAR

All vectors described herein comprising an extracellular portion of Factor VIII A2 or C2 subunit should be construed to be equally compatible with use of any Factor VIII extracellular portion. As such, use of the vectors described herein is exemplified by use of A2 or C2 subunit, but should be construed to be equally disclosed with respect to use of A1, B, A3, and C1 subunits.

For proof of concept as to specificity and functionality, a lentiviral vector plasmid is useful (e.g., pELPS-hFVIII-A2-BBz-T2A-mCherry, pELPS-hFVIII-C2-BBz-T2A-mCherry, pTRPE-hFVIII-A2-BBz, and pTRPE-hFVIII-C2-BBz), where BBz denotes 4-1BB CD3 zeta. This results in stable (permanent) expression in the host T cell. As an alternative approach, the encoding mRNA can be electroporated into the host cell, which would achieve the same therapeutic effect as the virally transduced T cells, but would not be permanent, since the mRNA would dilute out with cell division.

In one aspect, the invention includes a vector comprising an isolated nucleic acid sequence encoding a chimeric alloantigen receptor (CALLAR), wherein the isolated nucleic acid sequence comprises a nucleic acid sequence encoding an extracellular domain comprising an alloantigen or fragment thereof (such as a Factor VIII subunit), a nucleic acid sequence encoding a transmembrane domain, a nucleic acid sequence encoding an intracellular domain of a costimulatory molecule (such as 4-1BB), and a nucleic acid sequence encoding an intracellular signaling domain (such as CD3 zeta). In one embodiment, the vector comprises any of the isolated nucleic acid sequences encoding the CALLAR as described herein. In another embodiment, the vector comprises a plasmid vector, viral vector, retrotransposon (e.g. piggyback, sleeping beauty), site directed insertion vector (e.g. CRISPR, zinc finger nucleases, TALEN), or suicide expression vector, or other known vector in the art.

All constructs disclosed herein comprising different alloantigens and fragments thereof, can be incorporated into any lentiviral vector plasmid, other viral vectors, or RNA approved for use in human cells. In one embodiment, the vector is a viral vector, such as a lentiviral vector. In another embodiment, the vector is a RNA vector.

The production of the CALLAR can be verified by sequencing. Expression of the full length CALLAR protein may be verified using immunoblot, immunohistochemistry, flow cytometry or other technology well known and available in the art.

The present invention also provides a vector in which DNA encoding the CALLAR of the present invention is inserted. Vectors, including those derived from retroviruses such as lentivirus, are suitable tools to achieve long-term gene transfer since they allow long-term, stable integration of a transgene and its propagation in daughter cells. Lentiviral vectors have the added advantage over vectors derived from onco-retroviruses, such as murine leukemia viruses, in that they can transduce non-proliferating cells, such as hepatocytes. They also have the added advantage of resulting in low immunogenicity in the subject into which they are introduced.

The expression of natural or synthetic nucleic acids encoding CALLARs is typically achieved by operably linking a nucleic acid encoding the CALLAR polypeptide or portions thereof to a promoter, and incorporating the construct into an expression vector. The vector is one generally capable of replication in a mammalian cell, and/or also capable of integration into the cellular genome of the mammal. Typical vectors contain transcription and translation terminators, initiation sequences, and promoters useful for regulation of the expression of the desired nucleic acid sequence.

The nucleic acid can be cloned into any number of different types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

The expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

An example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, the elongation factor-1a promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of a CALLAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assessed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, NY).

Biological methods for introducing a polynucleotide of interest into a host cell include the use of DNA and RNA vectors. RNA vectors include vectors having a RNA promoter and/other relevant domains for production of a RNA transcript. Viral vectors, and especially retroviral vectors, have become the most widely used method for inserting genes into mammalian, e.g., human cells. Other viral vectors may be derived from lentivirus, poxviruses, herpes simplex virus, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

### Cells Comprising a CALLAR

In another aspect, the invention includes a genetically modified cell, such as a helper T cell, a cytotoxic T cell, a memory T cell, regulatory T cell, gamma delta T cell, a natural killer cell, a monocyte, a cytokine induced killer cell, a cell line thereof, and other effector cell that comprises the nucleic acid encoding the CALLAR described herein. In one embodiment, the genetically modified cell comprises an isolated nucleic acid sequence encoding a chimeric alloantigen receptor (CALLAR), wherein the isolated nucleic acid sequence comprises a nucleic acid sequence encoding an extracellular domain comprising an alloantigen or fragment thereof (such as a Factor VIII subunit), a nucleic acid sequence encoding a transmembrane domain, a nucleic acid sequence encoding an intracellular domain of a costimulatory molecule (such as 4-1BB), and a nucleic acid sequence encoding an intracellular signaling domain (such as CD3 zeta).

In another embodiment, the genetically modified cell comprises a CALLAR comprising an extracellular domain comprising an alloantigen or fragment thereof, a transmembrane domain, an intracellular domain of 4-1BB, and a CD3 zeta signaling domain. In another embodiment, the genetically modified cell comprises a CALLAR comprising an extracellular domain comprising A2 subunit of Factor VIII, a transmembrane domain, an intracellular domain of a costimulatory molecule, and an intracellular signaling domain.

In another embodiment, the cell expresses the CALLAR. In this embodiment, the cell has high affinity for alloantibodies expressed on B cells. As a result, the cell induces killing of B cells expressing the alloantibodies.

In another embodiment, the genetically modified cell is a T cell. In this embodiment, the T cell expresses the CALLAR described herein and the T cell has high affinity for Factor VIII alloantibodies expressed on B cells. As a result, the T cell induces killing of B cells expressing Factor VIII alloantibodies.

It is also useful for the T cell to have limited toxicity toward healthy cells and specificity to cells expressing alloantibodies. Such specificity prevents or reduces off-target toxicity that is prevalent in current therapies that are not specific for autoantibodies. In one embodiment the T cell has limited toxicity toward healthy cells.

The invention includes T cells, such as primary cells, expanded T cells derived from primary T cells, T cells derived from stem cells differentiated in vitro, T cell lines such as Jurkat cells, other sources of T cells, combinations thereof, and other effector cells.

The functional ability of CALLARs to bind to alloantibodies and sera, for example, but not limited to, hemophilia, may be assessed in a Jurkat reporter cell line, which would depend on activation of the CALLAR by binding to auto- and alloantibody (in response to which the activated cells fluoresce green due to an NFAT-GFP reporter construct contained therein). Such methods are useful and reliable qualitative measures for functional binding ability.

The CALLAR constructs described herein are compatible with VSV-G pseudotyped HIV-1 derived lentiviral particles and can be permanently expressed in primary human T cells from healthy donors using lentiviral transduction. Killing efficacy can be determined in a chromium based cell lysis assay or any similar assay known in the art.

Additional target cell lines can be produced as needed by expression of human monoclonal antibodies on the surface of K562 cells.

Sources of T Cells

Prior to expansion and genetic modification, T cells are obtained from a subject. Examples of subjects include humans, dogs, cats, mice, rats, and transgenic species thereof. T cells can be obtained from a number of sources, including skin, peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art, may be used. In certain embodiments of the present invention, T cells can be obtained from a unit of blood collected from a subject using any number of techniques known to the skilled artisan, such as Ficoll™ separation. In one preferred embodiment, cells from the circulating blood of an individual are obtained by apheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Again, surprisingly, initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA⁺, and CD45ROT cells, can be further isolated by positive or negative selection techniques. For example, in one embodiment, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In certain embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4⁺, CD25⁺, CD62L^{hi}, GITR⁺, and FoxP3⁺. Alternatively, in certain embodiments, T regulatory cells are depleted by anti-C25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8⁺ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4⁺ T cells express higher levels of CD28 and are more efficiently captured than CD8⁺ T cells in dilute concentrations. In one embodiment, the concentration of cells used is 5×10⁶/ml. In other embodiments, the concentration used can be from about 1×10⁵/ml to 1×10⁶/ml, and any integer value in between.

In other embodiments, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain embodiments, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one embodiment a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun. 73:316-321, 1991; Bierer et al., Curr. Opin. Immun. 5:763-773, 1993). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH. In another embodiment, the cells are isolated prior to and can be frozen for later use for treatment following B-cell ablative therapy, e.g., Rituxan.

In a further embodiment of the present invention, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

### Activation and Expansion of T Cells

T cells are activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the T cells of the invention are expanded by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4⁺ T cells or CD8⁺ T cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In certain embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In one embodiment, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the co-stimulatory signal is an anti-CD28 antibody or antigen-binding fragment thereof; and both agents are co-immobilized to the same bead in equivalent molecular amounts. In one embodiment, a 1:1 ratio of each antibody bound to the beads for CD4⁺ T cell expansion and T cell growth is used. In certain aspects of the present invention, a ratio of anti CD3:CD28 antibodies bound to the beads is used such that an increase in T cell expansion is observed as compared to the expansion observed using a ratio of 1:1. In one particular embodiment an increase of from about 1 to about 3 fold is observed as compared to the expansion observed using a ratio of 1:1. In one embodiment, the ratio of CD3:CD28 antibody bound to the beads ranges from 100:1 to 1:100 and all integer values there between. In one aspect of the present invention, more anti-CD28 antibody is bound to the particles than anti-CD3 antibody, i.e., the ratio of CD3:CD28 is less than one. In certain embodiments of the invention, the ratio of anti CD28 antibody to anti CD3 antibody bound to the beads is greater than 2:1. In one particular embodiment, a 1:100 CD3:CD28 ratio of antibody bound to beads is used. In another embodiment, a 1:75 CD3:CD28 ratio of antibody bound to beads is used. In a further embodiment, a 1:50 CD3:CD28 ratio of antibody bound to beads is used. In another embodiment, a 1:30 CD3:CD28 ratio of antibody bound to beads is used. In one preferred embodiment, a 1:10 CD3:CD28 ratio of antibody bound to beads is used. In another embodiment, a 1:3 CD3:CD28 ratio of antibody bound to the beads is used. In yet another embodiment, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain embodiments the ratio of cells to particles ranges from 1:100 to 100:1 and any integer values in-between and in further embodiments the ratio comprises 1:9 to 9:1 and any integer values in between, can also be used to stimulate T cells. The ratio of anti-CD3- and anti-CD28-coupled particles to T cells that result in T cell stimulation can vary as noted above, however certain preferred values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one preferred ratio being at least 1:1 particles per T cell. In one embodiment, a ratio of particles to cells of 1:1 or less is used. In one particular embodiment, a preferred particle:cell ratio is 1:5. In further embodiments, the ratio of particles to cells can be varied depending on the day of stimulation. For example, in one embodiment, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular embodiment, the ratio of particles to cells is 1:1 on the first day of stimulation and adjusted to 1:5 on the third and fifth days of stimulation. In another embodiment, particles are added on a daily or every other day basis to a final ratio of 1:1 on the first day, and 1:5 on the third and fifth days of stimulation. In another embodiment, the ratio of particles to cells is 2:1 on the first day of stimulation and adjusted to 1:10 on the third and fifth days of stimulation. In another embodiment, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type.

In further embodiments of the present invention, the cells, such as T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one embodiment the cells (for example, 10⁴ to 10⁹ T cells) and beads (for example, DYNABEADS® M-450 CD3/CD28 T paramagnetic beads at a ratio of 1:1) are combined in a buffer, for example PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present invention. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/ml is used. In another embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain embodiments. For example, using high concentration of cells allows more efficient selection of CD8+ T cells that normally have weaker CD28 expression.

In one embodiment of the present invention, the mixture may be cultured for several hours (about 3 hours) to about 14 days or any hourly integer value in between. In another embodiment, the mixture may be cultured for 21 days. In one embodiment of the invention the beads and the T cells are cultured together for about eight days. In another embodiment, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

T cells that have been exposed to varied stimulation times may exhibit different characteristics. For example, typical blood or apheresed peripheral blood mononuclear cell products have a helper T cell population (TH, CD4⁺) that is greater than the cytotoxic or suppressor T cell population (Tc, CD8⁺). Ex vivo expansion of T cells by stimulating CD3 and CD28 receptors produces a population of T cells that prior to about days 8-9 consists predominately of TH cells, while after about days 8-9, the population of T cells comprises an increasingly greater population of Tc cells. Accordingly, depending on the purpose of treatment, infusing a subject with a T cell population comprising predominately of TH cells may be advantageous. Similarly, if an antigen-specific subset of Tc cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

### Therapy

The present invention also provides methods for preventing, treating and/or managing a disorder associated with Factor VIII antibody-expressing cells (e.g., anti-FVIII antibodies in a subject with hemophila treated with FVIII replacement therapy). Non-limiting examples of disorders associated with auto- and/or alloantibody-expressing cells include hemophilia and related disorders. In one embodiment, the subject is a human.

In one aspect, the invention includes a method for treating a disorder associated with FVIII antibodies in a subject with hemophilia. The method comprises administering to the subject an effective amount of a genetically modified T cell comprising an isolated nucleic acid sequence encoding a chimeric alloantigen receptor (CALLAR), wherein the isolated nucleic acid sequence comprises a nucleic acid sequence encoding an alloantigen or fragment thereof, a nucleic acid sequence encoding a transmembrane domain, a nucleic acid sequence encoding an intracellular signaling domain of 4-1BB, and a nucleic acid sequence encoding a CD3 zeta signaling domain, thereby treating the antibodies in the subject with hemophilia.

In another aspect, the invention includes a method for treating a disorder associated with FVIII antibodies in a subject with hemophilia. The method comprises administering to the subject an effective amount of a genetically modified T cell comprising an isolated nucleic acid sequence encoding a chimeric alloantigen receptor (CALLAR), wherein the isolated nucleic acid sequence comprises a nucleic acid sequence encoding A2 subunit of factor VIII, a nucleic acid sequence encoding a transmembrane domain, a nucleic acid sequence encoding an intracellular domain of a costimulatory molecule, and a nucleic acid sequence encoding an intracellular signaling domain, thereby treating the a disorder associated with FVIII antibodies in the subject with hemophilia.

The methods of the invention comprise administering to a subject in need a CALLAR T cell of the invention that binds to the auto- and alloantibody-expressing cell. In one embodiment, the subject undergoes plasmapheresis or another clinical treatment to remove or decrease antibodies in the subject's serum. The method to remove or decrease serum antibodies, such as auto- and/or alloantibodies, may include chemical or other methods known in the art. The treatment method may be specific to the auto- and/or alloantibody or generalized for any antibody. In one embodiment, the subject is a human. Non-limiting examples of diseases associated with auto- and alloantibody-expressing cells include FVIII antibodies in subjects with hemophilia treated with FVIII replacement therapy, and the like.

In the methods of treatment described herein, T cells isolated from a subject can be modified to express the appropriate CALLAR, expanded ex vivo and then reinfused into the subject. The modified T cells recognize target cells, such as factor VIII specific B cells, and become activated, resulting in killing of the alloimmune target cells.

In order to monitor CALLAR-expressing cells in vitro, in situ, or in vivo, CALLAR cells can further express a detectable marker. When the CALLAR binds the target, the detectable marker is activated and expressed, which can be detected by assays known in the art, such as flow cytometry.

Without wishing to be bound by any particular theory, the anti-FVIII antibody immune response elicited by the CALLAR-modified T cells may be an active or a passive immune response. In yet another embodiment, the modified T cell targets a B cell. For example, the target antibody expressing B cells may be susceptible to indirect destruction by CALLAR-redirected T cells that have previously reacted against adjacent antibody-expressing cells.

In one embodiment, the fully-human CALLAR-genetically modified T cells of the invention may be used as a type of vaccine for ex vivo immunization and/or in vivo therapy in a mammal. In one embodiment, the mammal is a human.

With respect to ex vivo immunization, one of the following may occur in vitro prior to administering the cell into a mammal: i) expansion of the cells, ii) introducing a nucleic acid encoding a CALLAR to the cells or iii) cryopreservation of the cells.

Ex vivo procedures are well known in the art and are discussed more fully below. Briefly, cells are isolated from a mammal (e.g., a human) and genetically modified (i.e., transduced or transfected in vitro) with a vector expressing a CALLAR disclosed herein. The CALLAR-modified cell can be administered to a mammalian recipient to provide a therapeutic benefit. The mammalian recipient may be a human and the CALLAR-modified cell may be autologous with respect to the recipient. Alternatively, the cells can be allogeneic, syngeneic or xenogeneic with respect to the recipient.

One example of a procedure for ex vivo expansion of hematopoietic stem and progenitor cells that can be applied to the cells of the present invention is described in U.S. Pat. No. 5,199,942, incorporated herein by reference. Other suitable methods are known in the art and therefore the present invention should not be construed to be limited to any particular method of ex vivo expansion of the cells. Briefly, ex vivo culture and expansion of T cells generally comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

In addition to using a cell-based vaccine in terms of ex vivo immunization, the present invention also includes compositions and methods for in vivo immunization to elicit an immune response directed against an antigen in a patient.

Generally, the cells described herein may be utilized in the treatment and prevention of diseases that arise in individuals who are immunocompromised. In particular, the CALLAR-modified T cells of the invention are used in the treatment of diseases, disorders and conditions associated with expression of antibodies. In certain embodiments, the cells of the invention are used in the treatment of patients at risk for developing diseases, disorders and conditions associated with expression of antibodies. Thus, the present invention provides methods for the treatment or prevention of diseases, disorders and conditions associated with expression of antibodies, such as FVIII antibodies in subjects with hemophilia treated with FVIII replacement therapy, comprising administering to a subject in need thereof, a therapeutically effective amount of the CALLAR-modified T cells of the invention.

The CALLAR-modified T cells of the present invention may be administered either alone, or as a pharmaceutical composition in combination with diluents and/or with other components such as IL-2 or other cytokines or cell populations. Briefly, pharmaceutical compositions of the present invention may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are in one aspect formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

It can generally be stated that a pharmaceutical composition comprising the T cells described herein may be administered at a dosage of 10⁴ to 10⁹ cells/kg body weight, in some instances 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, activated T cells are administered to a subject. Subsequent to administration, blood is redrawn or apheresis is performed, and T cells are activated and expanded therefrom using the methods described here, and are then reinfused back into the patient. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocol, may select out certain populations of T cells.

Administration of the cells of the invention may be carried out using any convenient means, including by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In one embodiment, the T cell compositions of the present invention are administered to a patient by intradermal or subcutaneous injection. In another embodiment, the T cell compositions of the present invention are administered by i.v. injection. The compositions of T cells may be injected directly into a tumor, lymph node, or site of infection.

In certain embodiments of the present invention, cells are activated and expanded using the methods described herein, or other methods known in the art where T cells are expanded to therapeutic levels, and administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In further embodiments, the T cells of the invention may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludarabine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun. 73:316-321, 1991; Bierer et al., Curr. Opin. Immun. 5:763-773, 1993). In a further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The dosage of the above treatments to be administered to a patient will vary with the precise nature of the condition being treated and the recipient of the treatment. The scaling of dosages for human administration can be performed according to art-accepted practices. The dose for CAMPATH, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

### Experimental Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

The Materials and Methods used in the performance of the experiments disclosed herein are now described.

Detection of A2 and C2 CALLARs.

T cells were activated with CD3/28 beads for 24 hrs followed by lentiviral transduction of an A2-CALLAR or C2-CALLAR utilizing the 4-1BB and CD3 zeta signaling domains (A2bbz and C2bbz, respectively). Lentiviral vectors expressing A2- or C2-CALLAR constructs in which mCherry was fused to the c-terminus of the zeta domain (A2bbz-mCh or C2bbz-mCh, respectively) were also generated and used for transduction. FMC63bbz CAR (CD19 CAR) was used as a control. T cells were stained with either A2 or C2 specific antibodies as indicated on day 5 following transduction to detect expression of the A2 and C2 containing CALLARs. Protein L was used to stain for the FMC63bbz CAR.

Activation of A2 and C2 CALLARs.

In some embodiments, T cells transduced with indicated CAR or CALLAR were plated on microwells coated with OKT3 (for polyclonal T cell activation), anti-A2 or anti-C2. Supernatants were harvest at 24 hours, and IFN-γ was measured by ELISA. In some embodiments, T cells were mixed at varying T cell (Effector) to target cell ratios (E:T ratios) to determine cytotoxicity and cytokine production upon binding of the CALLAR or CAR expressed on the T cell to cognate ligand expressed on the target cell. In some experiments, the Nalm-6 B-cell acute lymphoblastic leukemia cell line was engineered to express either A2 specific surface immunoglobulin or C2-specific surface immunoglobulin generated using murine monoclonal antibody-derived variable domain sequences to these respective domains.

The results of the experiments are now described.

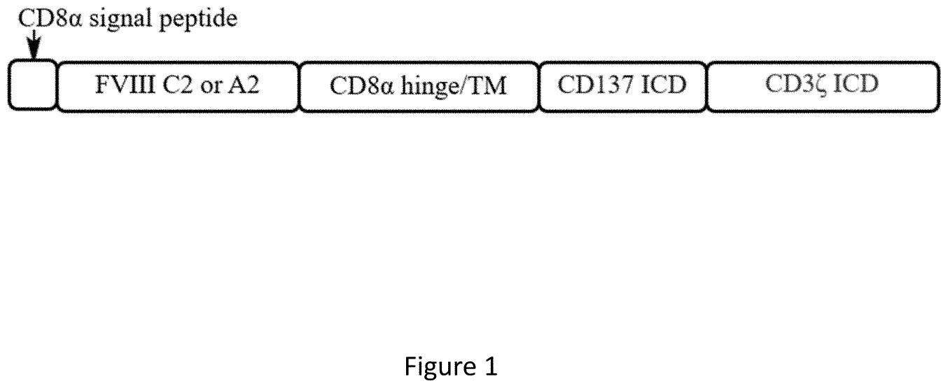

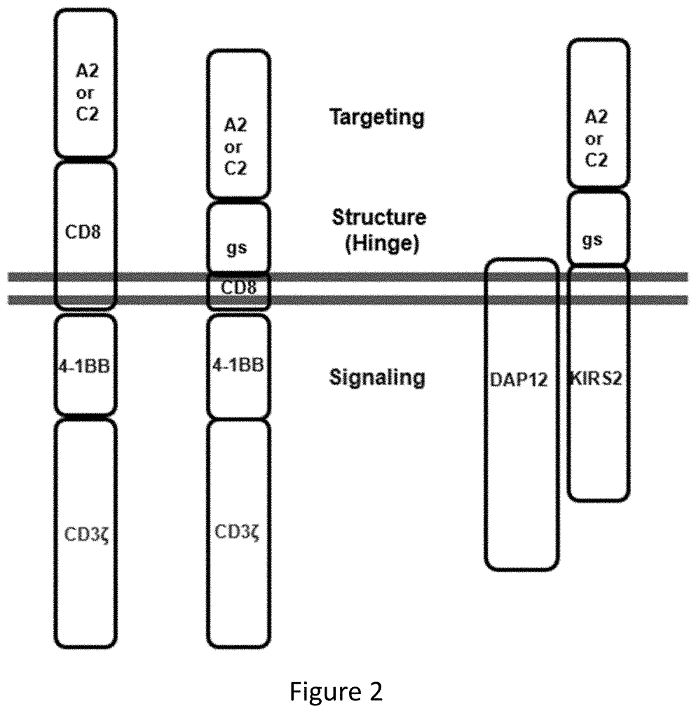

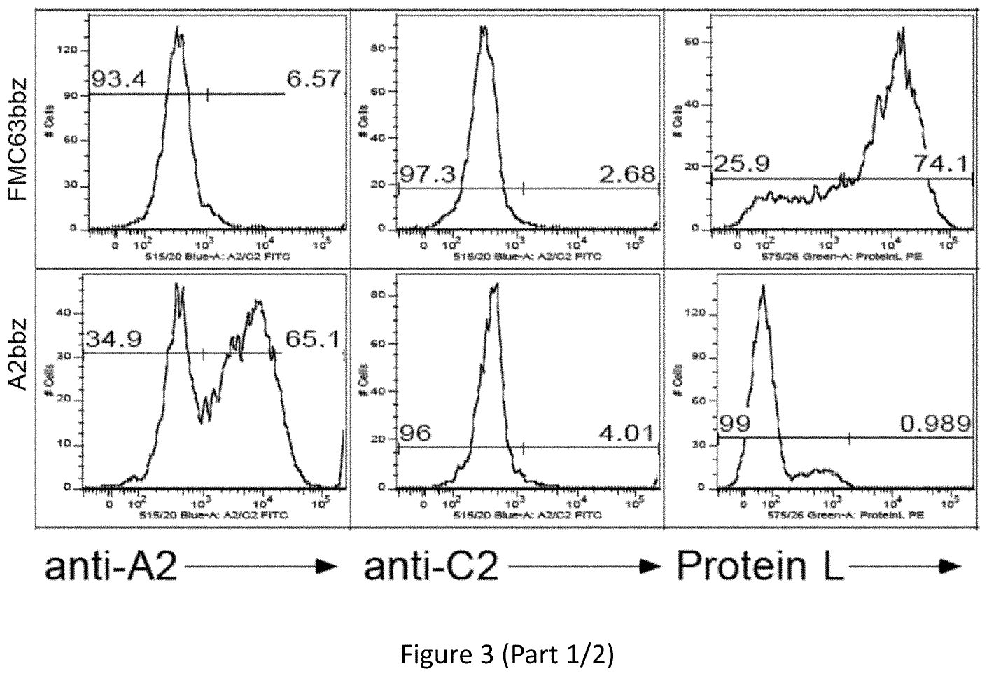

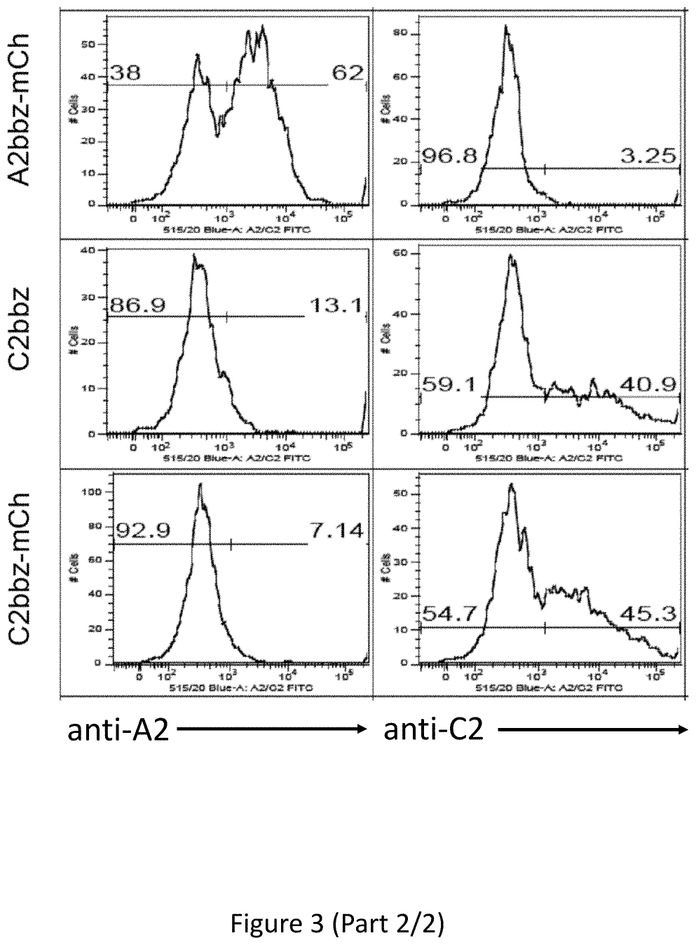

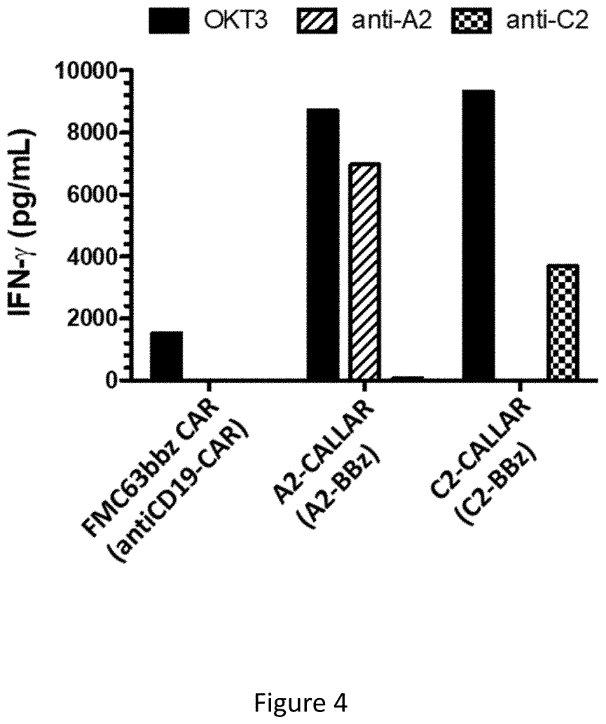

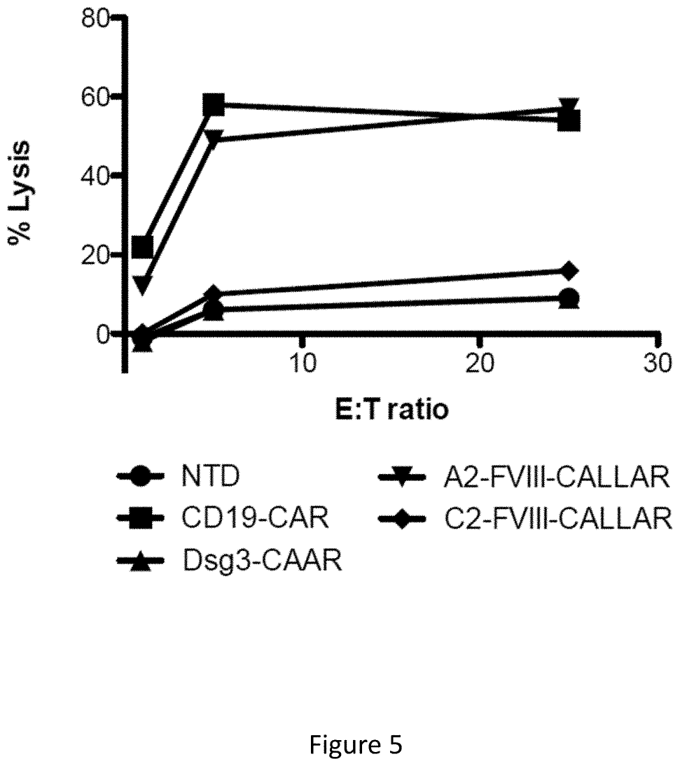

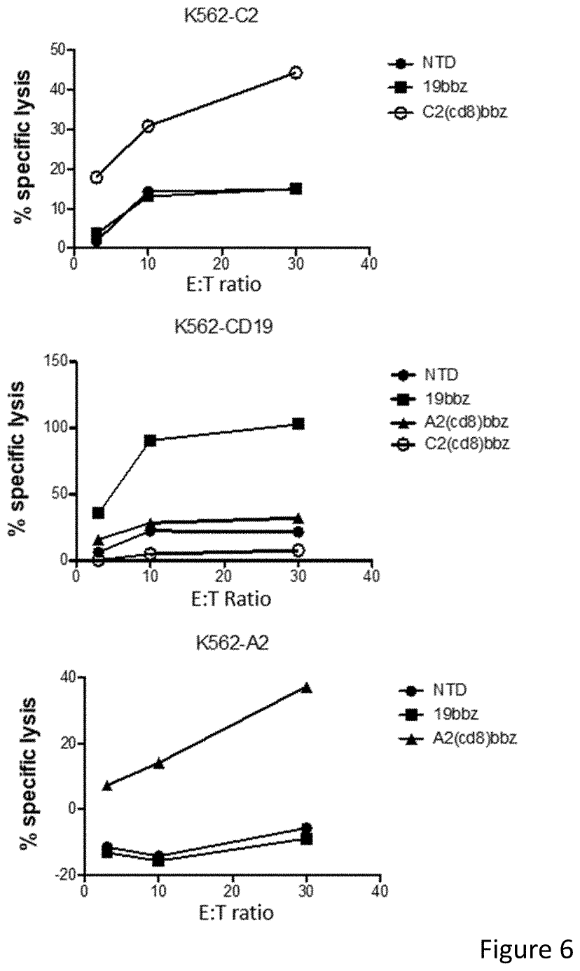

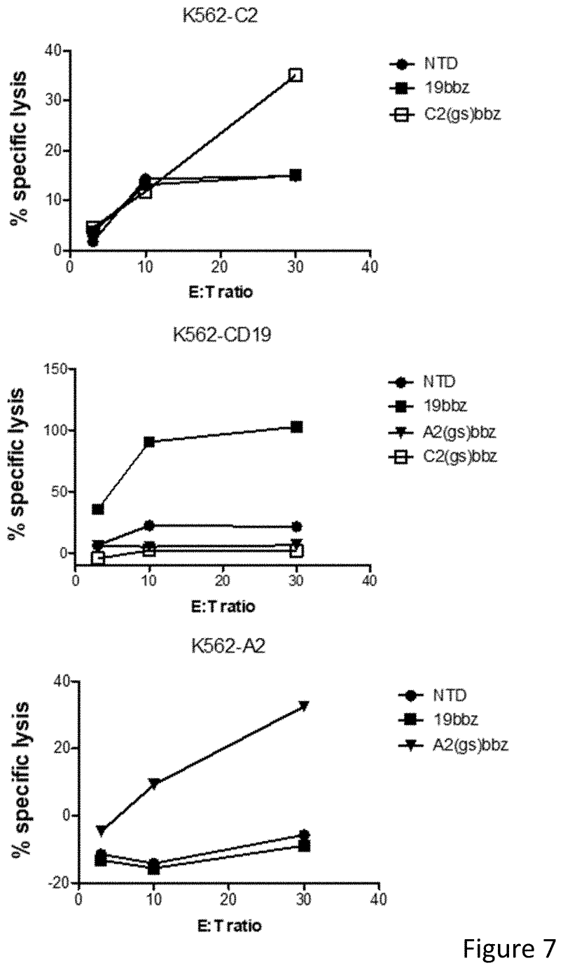

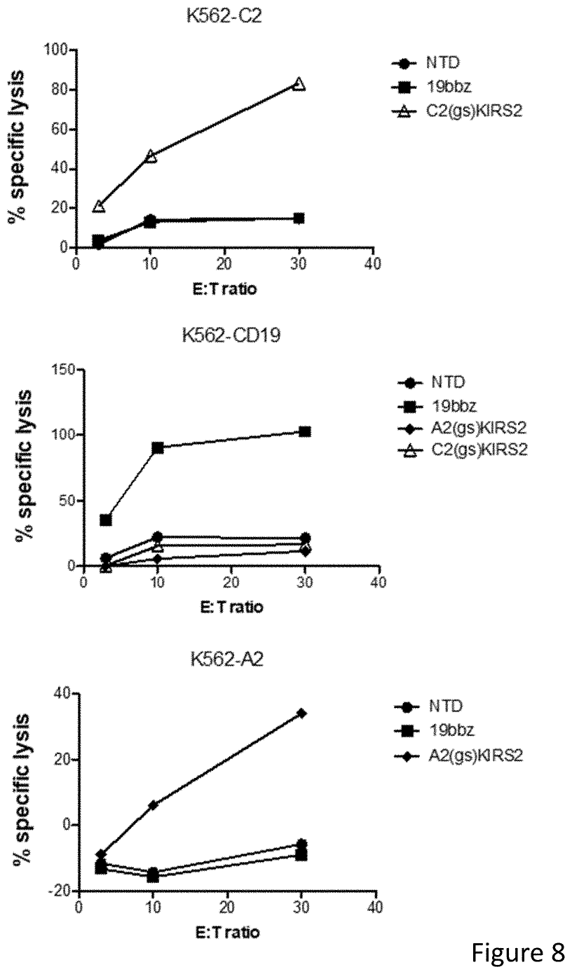

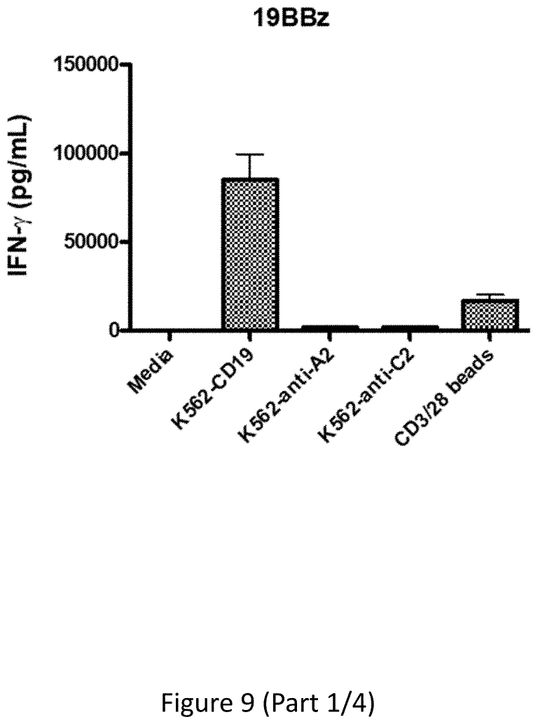

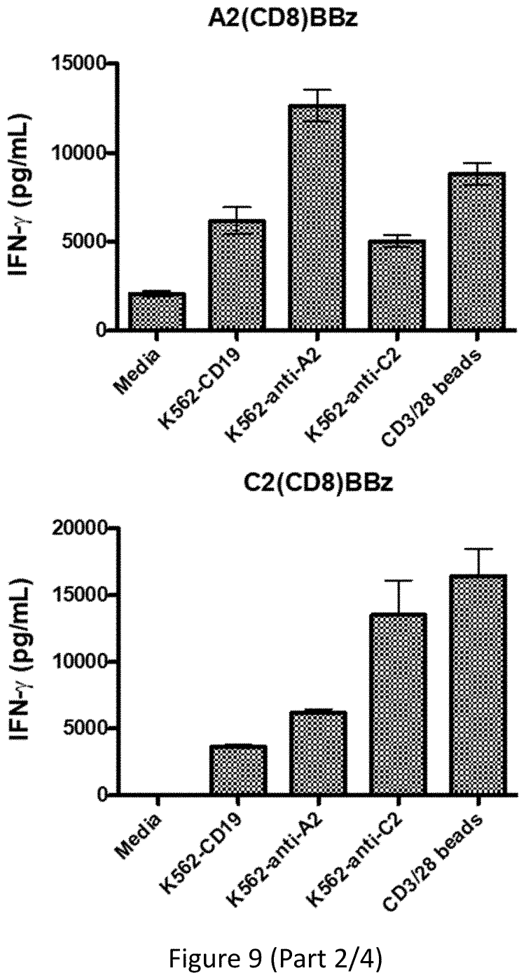

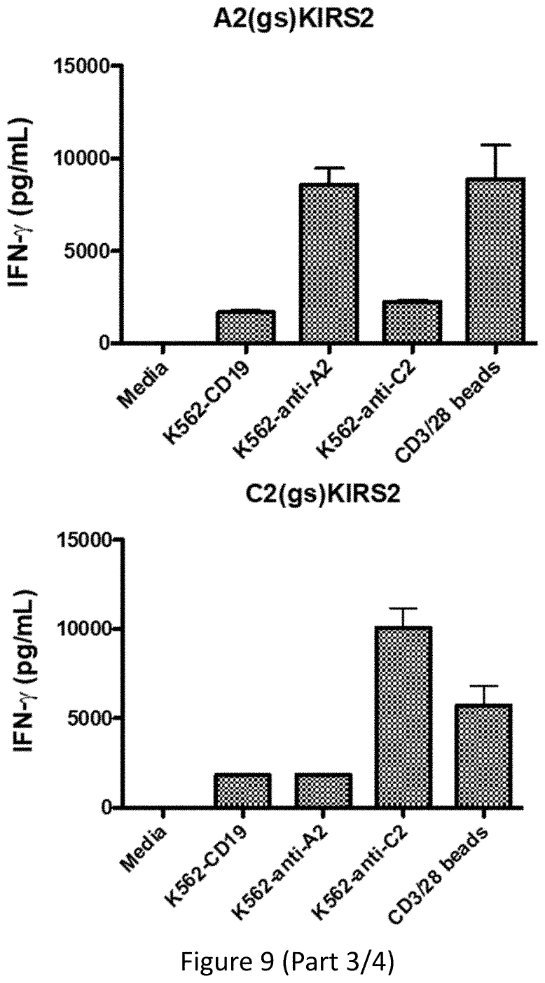

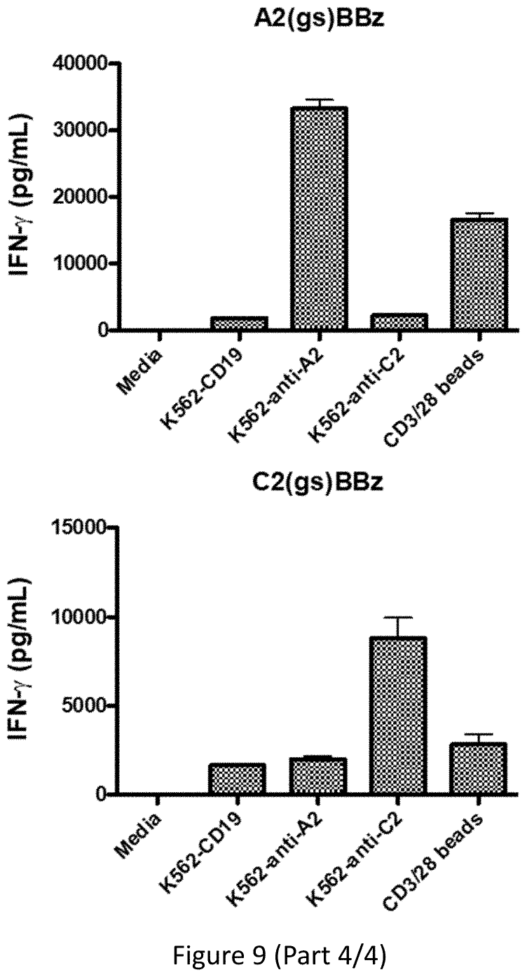

Chimeric molecules were designed to express FVIII epitopes derived from human FVIII that are linked to a transmembrane domain and cytoplasmic signaling domains that activate T cells and trigger their cytotoxic function. Non-limiting examples of possible designs are shown schematically in FIGS. 1 and 2. The chimeric molecules are named CALLARs (Chimeric ALLoAntigen Receptors) to distinguish them from traditional chimeric antigen receptors or CARs using an scFv for receptor targeting. The initial CALLARs incorporate the A2 and C2 domains from human FVIII since most inhibitory antibodies bind to epitopes in one of these two domains. When these CALLARs are introduced into human T cells by genetic modification (e.g. lentiviral vectors), these CALLAR-modified T cells were activated and killed B cells and plasma cells expressing surface immunoglobulin (sIg) that bound to either the A2 or C2 domains for FVIII. The modified T cells are expected to eliminate FVIII-specific B cells in vivo leading to the eradication of FVIII inhibitory antibodies. The KIR-based CALLAR (FIG. 2, right side) can trigger robust antigen-specific proliferation and effector function in vitro when introduced into human T cells with DAP12. In some embodiments, T cells are genetically modified to comprise a CALLAR comprising a chimeric KIR generated by fusing the FVIII domain with the transmembrane and short cytoplasmic domain of a KIR, e.g., KIRS2, KIR2DS2, that is co-expressed with DAP12. In some embodiments, the CALLAR comprises A2 or C2 domain of FVIII that is connected via a CD8alpha-derived extracellular hinge. In some embodiments, the CALLAR comprises A2 or C2 domain of FVIII that is connected via glycine-serine derived extracellular hinge such as Gly-Gly-Gly-Gly-Ser-Gly-Gly-Gly-Gly-Ser. In some embodiments, the genetically modified T cells are administered to a subject having FVIII antibodies. Sequences of some portions of the chimeric molecules useful in the present invention are provided as SEQ ID NOs: 21-28.

Surface expression of A2 and C2 CALLAR on human T cells was analyzed (FIG. 3). Lentiviral vector transduction of CD3/28-activated T cells demonstrated that both the A2-specific and C2-specific CALLARs were expressed on the surface of T cells. T cells were activated with CD3/28 beads for 24 hrs followed by lentiviral transduction of an A2-CALLAR or C2-CALLAR utilizing the 4-1BB and Zeta signaling domains (A2bbz and C2bbz, respectively). Lentiviral vectors expressing A2- or C2-CALLAR constructs (A2bbz-mCh or C2bbz-mCh) were also generated and used for transduction. FMC63bbz CAR (anti-CD19 CAR) was used as a control. T cells were stained with either an A2 or C2 specific antibodies as indicated on day 5 following transduction to detect expression of the A2 and C2 containing CALLARs. Protein L was used to stain for the FMC63bbz CAR. Flow cytometry was used to analyze A2 and C2-based CARs on primary T-cells. Fresh isolated human T cells from healthy donors were transduced with lentiviral vector supernatants encoding the following CARs: FMC63-bbz, A2-bbz, and C2-bbz. A2bbz-mCh and C2bbz-mCh represent T cells transduced with lentiviral vectors encoding a bi-cistronic construct for expression of the respective CAR and mCherry as separate proteins. CAR expression was evaluated by flow cytometry. Briefly, T cells were cultured in RPMI 1640 medium with 10% FBS and stimulated with anti-CD3/anti-CD28 Dynabeads (invitrogen). 24 hrs after stimulation, T cells were transduced with the CAR lentiviral vector supernatants. 6-8 days after lentiviral transduction T cells were stained with biotinylated Protein L antibody followed by strepavidin PE (BD Biosciences), anti-A2 followed by or goat-anti mouse-FITC (Jackson ImmunoResearch), or anti-C2 followed by or goat-anti mouse-FITC (Jackson ImmunoResearch) as indicated. CAR expression was evaluated by flow cytometry (LSR-II, BD). Flow cytometry analysis was carried out by using Flowjo (Tree Star Inc). After transduction it was observed that A2 and C2 domain-based CARs were efficiently expressed on the cell surface of the transduced T cells.

T cells expressing these CALLARs secreted IFN-gamma with the A2-CALLAR responding to anti-A2 antibody, and not anti-C2 antibody. As expected, C2-CALLAR T cells responded to anti-C2 antibody, but not anti-A2 antibody. Control T cells expressing a CD19-specific standard CAR did not respond to either anti-A2 or anti-C2. However, all CALLAR or CAR T cells responded to polyclonal stimulation with OKT3 (FIG. 4). T cells transduced with indicated CAR or CALLAR were plated on microwells coated with OKT3 (for polyclonal T cell activation), anti-A2 or anti-C2. Supernatants were harvested at 24 hours, and IFN-γ was measured by ELISA. T cells were transduced with lentiviral vectors encoding an anti-CD19 CAR, an A2-domain containing chimeric alloantibody receptor (A2-BBz) or a C2-domain containing receptor (C2-BBz). After 7-9 days of culture, the T cells were transferred to polystyrene multi-well plates pre-coated with antibodies to CD3 (clone OKT3), anti-A2 (Green Mountain Antibodies), and anti-C2 (Green Mountain Antibodies). Following 24 hours incubation at 37 degrees C., supernatants were harvested for interferon-gamma (IFNγ) analysis by ELISA. Results illustrate that all T cells are capable of producing IFNγ following activation by anti-CD3 antibody. Only A2-BBz transduced T cells produce IFNγ in response to A2-specific antibody. Only C2-BBz transduced T cells produce IFNγ in response to C2-specific antibody.

CD19+ Nalm6 cells were engineered to express FVIII-specific chimeric immunoglobulin in a CALLARs model system for antigen-specific B cells (FIG. 5). Human peripheral blood T cells were transduced with A2-FVIII-CALLARs, C2-FVIII-CALLARs, Dsg3-CAAR or CD19-CAR (controls) or non-transduced T cells (NTD). The T cells were mixed with Nalm6 cells engineered to express surface immunoglobulin specific for the A2 domain of FVIII at varying effector to target (E:T) ratios. Percent specific lysis was measured by a 51Cr release assay at 16 hours.

Studies to determine the ability of these CALLARs to respond to surface immunoglobulin are described elsewhere herein. In some embodiments, the K562 cells may co-express CD79a and CD79b.

T cells were transduced with lentiviral vectors encoding an anti-CD19 CAR (19BBz), an A2-domain containing chimeric alloantibody receptor with a CD8 extracellular spacer (A2(cd8)BBz) or a C2-domain containing receptor with the same CD8 spacer (C2(cd8)BBz) (FIG. 6). After 7-9 days of culture, the cytotoxic activity of the transduced T cells was assessed by a 4-hour ⁵¹Cr-release assay using K562 target cells that were engineered to express CD19 (K562-CD19), an A2 specific surface immunoglobulin (K562-A2) or a C2-specific surface immunoglobulin (K562-C2) and varying effector to target cell ratio (E:T ratio) as indicated. 19BBz-expressing T cells only show cytotoxicity towards the CD19+ target K562 cells. A2(cd8)BBz transduced T cells only mediate lysis of K562 target cells expressing anti-A2 surface immunoglobulin. C2(cd8)BBz transduced T cells only mediate lysis of K562 target cells expressing anti-C2 surface immunoglobulin.

T cells were transduced with lentiviral vectors encoding an anti-CD19 CAR (19BBz), an A2-domain containing chimeric alloantibody receptor with a synthetic (Gly)₄-Ser extracellular spacer (A2(gs)BBz) or a C2-domain containing receptor with the same (Gly)₄-Ser spacer (C2(gs)BBz) (FIG. 7). After 7-9 days of culture, the cytotoxic activity of the transduced T cells was assessed by a 4-hour ⁵¹Cr-release assay using K562 target cells that were engineered to express CD19 (K562-CD19), an A2 specific surface immunoglobulin (K562-A2) or a C2-specific surface immunoglobulin (K562-C2) and varying effector to target cell ratio (E:T ratio) as indicated. 19BBz-expressing T cells only show cytotoxicity towards the CD19+ target K562 cells. A2(gs)BBz transduced T cells only mediate lysis of K562 target cells expressing anti-A2 surface immunoglobulin. C2(gs)BBz transduced T cells only mediate lysis of K562 target cells expressing anti-C2 surface immunoglobulin.

T cells were transduced with lentiviral vectors encoding an anti-CD19 CAR (19BBz), an A2-domain containing chimeric alloantibody receptor with KIR/DAP12 signaling (A2(gs)KIRS2) or a C2-domain containing receptor with the same KIR/DAP12 signaling (C2(gs)KIRS2) (FIG. 8). After 7-9 days of culture, the cytotoxic activity of the transduced T cells was assessed by a 4-hour ⁵¹Cr-release assay using K562 target cells that were engineered to express CD19 (K562-CD19), an A2 specific surface immunoglobulin (K562-A2) or a C2-specific surface immunoglobulin (K562-C2) and varying effector to target cell ratio (E:T ratio) as indicated. 19BBz-expressing T cells only show cytotoxicity towards the CD19+ target K562 cells. A2(gs)KIRS2-transduced T cells only mediate lysis of K562 target cells expressing anti-A2 surface immunoglobulin. C2(gs)KIRS2-transduced T cells only mediate lysis of K562 target cells expressing anti-C2 surface immunoglobulin.

T cells were transduced with lentiviral vectors encoding an anti-CD19 CAR (19BBz), A2-domain containing chimeric alloantibody receptors with a CD8 extracellular spacer (A2(cd8)BBz), a synthetic (Gly)₄-Ser (A2(gs)BBz) or with KIR/DAP12 signaling (A2(gs)KIRS2), or C2-domain containing receptor with the same CD8 spacer (C2(cd8)BBz), synthetic (Gly)₄-Ser (C2(gs)BBz) or with KIR/DAP12 signaling (C2(gs)KIRS2) (FIG. 9). After 7-9 days of culture, the transduced T cells were mixed at a 1:1 ratio with K562 target cells that were engineered to express CD19 (K562-CD19), an A2 specific surface immunoglobulin (K562-A2) or a C2-specific surface immunoglobulin (K562-C2). Stimulator microbeads coated with anti-CD3 and anti-CD28 (CD3/28 beads, Dynal) or media alone were used as an additional positive and negative controls, respectively. Following 24 hours incubation at 37 degrees C., supernatants were harvested for interferon-gamma (IFNγ) analysis by ELISA. 19BBz-expressing T cells only show enhanced IFNγ production in response to CD19+ target K562 cells or CD3/28 beads. A2(cd8)BBz, A2(gs)BBz and A2(gs)KIRS2 T cells show enhanced IFNγ production in response to K562 target cells expressing anti-A2 surface immunoglobulin or positive control CD3/28 beads. C2(cd8)BBz, C2(gs)BBz and C2(gs)KIRS2 T cells show enhanced IFNγ production in response to K562 target cells expressing anti-C2 surface immunoglobulin or positive control CD3/28 beads.

Additional studies include examining the extracellular hinge domain to determine the optimal structure for A2 and C2. Further, analysis of activation by anti-A2 and anti-C2 antibodies will determine how broadly CALLARs respond to antibodies across different epitopes. A2 and C2 may have the potential to interact weakly with binding partners for intact FVIII, such as von Willebrand Factor (vWF), phospholipids and platelets.

In some embodiments, this system provides a robust method for manipulating B-cells and plasma cells to create tolerance to functionally allogeneic enzymes like FVIII in hemophila A.

SEQ ID NOS: 13-28

pELPS-hFVIII-A2-BBz-T2A-mCherry
(SEQ ID NO: 13)
GATCTATGGA GTTTGGGCTG AGCTGGCTTT TTCTTGTGGC TATTTTAAAA GGTGTCCAGT GCGGATCCTC AGTTGCCAAG
AAGCATCCTA AAACTTGGGT ACATTACATT GCTGCTGAAG AGGAGGACTG GGACTATGCT CCCTTAGTCC TCGCCCCCGA
TGACAGAAGT TATAAAAGTC AATATTTGAA CAATGGCCCT CAGCGGATTG GTAGGAAGTA CAAAAAAGTC CGATTTATGG
CATACACAGA TGAAACCTTT AAGACTCGTG AAGCTATTCA GCATGAATCA GGAATCTTGG GACCTTTACT TTATGGGGAA
GTTGGAGACA CACTGTTGAT TATATTTAAG AATCAAGCAA GCAGACCATA TAACATCTAC CCTCACGGAA TCACTGATGT
CCGTCCTTTG TATTCAAGGA GATTACCAAA AGGTGTAAAA CATTTGAAGG ATTTTCCAAT TCTGCCAGGA GAAATATTCA
AATATAAATG GACAGTGACT GTAGAAGATG GGCCAACTAA ATCAGATCCT CGGTGCCTGA CCCGCTATTA CTCTAGTTTC
GTTAATATGG AGAGAGATCT AGCTTCAGGA CTCATTGGCC CTCTCCTCAT CTGCTACAAA GAATCTGTAG ATCAAAGAGG
AAACCAGATA ATGTCAGACA AGAGGAATGT CATCCTGTTT TCTGTATTTG ATGAGAACCG AAGCTGGTAC CTCACAGAGA
ATATACAACG CTTTCTCCCC AATCCAGCTG GAGTGCAGCT TGAAGATCCA GAGTTCCAAG CCTCCAACAT CATGCACAGC
ATCAATGGCT ATGTTTTTGA TAGTTTGCAG TTGTCAGTTT GTTTGCATGA GGTGGCATAC TGGTACATTC TAAGCATTGG
AGCACAGACT GACTTCCTTT CTGTCTTCTT CTCTGGATAT ACCTTCAAAC ACAAAATGGT CTATGAAGAC ACACTCACCC
TATTCCCATT CTCAGGAGAA ACTGTCTTCA TGTCGATGGA AAACCCAGGT CTATGGATTC TGGGGTGCCA CAACTCAGAC
TTTCGGAACA GAGGCATGAC CGCCTTACTG AAGGTTTCTA GTTGTGACAA GAACACTGGT GATTATTACG AGGACAGTTA
TGAAGATATT TCAGCATACT TGCTGAGTAA AAACAATGCC ATTGAACCAA GAGCTAGCAC CACGACGCCA GCGCCGCGAC
CACCAACACC GGCGCCCACC ATCGCGTCGC AGCCCCTGTC CCTGCGCCCA GAGGCGTGCC GGCCAGCGGC GGGGGGCGCA
GTGCACACGA GGGGGCTGGA CTTCGCCTGT GATTCCGGAA TCTACATCTG GGCCCCTCTG GCCGGCACCT GTGGCGTGCT
GCTGCTGTCC CTGGTCATCA CCCTGTACTG CAAGCGGGGC AGAAAGAAGC TGCTGTACAT CTTCAAGCAG CCCTTCATGC
GGCCTGTGCA GACCACACAG GAAGAGGACG GCTGTAGCTG TAGATTCCCC GAGGAAGAGG AAGGCGGCTG CGAGCTGAGA
GTGAAGTTCA GCAGAAGCGC CGACGCCCCT GCCTATCAGC AGGGCCAGAA CCAGCTGTAC AACGAGCTGA ACCTGGGCAG
ACGGGAGGAA TACGACGTGC TGGACAAGAG AAGAGGCCGG GACCCTGAGA TGGGCGGCAA GCCCAGACGG AAGAACCCCC
AGGAAGGCCT GTATAACGAA CTGCAGAAAG ACAAGATGGC CGAGGCCTAC AGCGAGATCG GCATGAAGGG CGAGCGGAGA
AGAGGCAAGG GCCATGACGG CCTGTACCAG GGCCTGAGCA CCGCCACCAA GGACACCTAC GACGCCCTGC ACATGCAGGC
CCTGCCTCCA AGAGGCAGCG GAGAGGGCAG AGGAAGTCTT CTAACATGCG GTGACGTGGA GGAGAATCCC GGCCCTACGC
GTATGGTGAG CAAGGGCGAG GAGGATAACA TGGCCATCAT CAAGGAGTTC ATGCGCTTCA AGGTGCACAT GGAGGGCTCC
GTGAACGGCC ACGAGTTCGA GATCGAGGGC GAGGGCGAGG GCCGCCCCTA CGAGGGCACC CAGACCGCCA AGCTGAAGGT
GACCAAGGGT GGCCCCCTGC CCTTCGCCTG GGACATCCTG TCCCCTCAGT TCATGTACGG CTCCAAGGCC TACGTGAAGC
ACCCCGCCGA CATCCCCGAC TACTTGAAGC TGTCCTTCCC CGAGGGCTTC AAGTGGGAGC GCGTGATGAA CTTCGAGGAC
GGCGGCGTGG TGACCGTGAC CCAGGACTCC TCCCTGCAGG ACGGCGAGTT CATCTACAAG GTGAAGCTGC GCGGCACCAA
CTTCCCCTCC GACGGCCCCG TAATGCAGAA GAAGACCATG GGCTGGGAGG CCTCCTCCGA GCGGATGTAC CCCGAGGACG
GCGCCCTGAA GGGCGAGATC AAGCAGAGGC TGAAGCTGAA GGACGGCGGC CACTACGACG CTGAGGTCAA GACCACCTAC
AAGGCCAAGA AGCCCGTGCA GCTGCCCGGC GCCTACAACG TCAACATCAA GTTGGACATC ACCTCCCACA ACGAGGACTA
CACCATCGTG GAACAGTACG AACGCGCCGA GGGCCGCCAC TCCACCGGCG GCATGGACGA GCTGTACAAG TAGGTCGACA
ATCAACCTCT GGATTACAAA ATTTGTGAAA GATTGACTGG TATTCTTAAC TATGTTGCTC CTTTTACGCT ATGTGGATAC
GCTGCTTTAA TGCCTTTGTA TCATGCTATT GCTTCCCGTA TGGCTTTCAT TTTCTCCTCC TTGTATAAAT CCTGGTTGCT
GTCTCTTTAT GAGGAGTTGT GGCCCGTTGT CAGGCAACGT GGCGTGGTGT GCACTGTGTT TGCTGACGCA ACCCCCACTG
GTTGGGGCAT TGCCACCACC TGTCAGCTCC TTTCCGGGAC TTTCGCTTTC CCCCTCCCTA TTGCCACGGC GGAACTCATC
GCCGCCTGCC TTGCCCGCTG CTGGACAGGG GCTCGGCTGT TGGGCACTGA CAATTCCGTG GTGTTGTCGG GGAAGCTGAC
GTCCTTTCCA TGGCTGCTCG CCTGTGTTGC CACCTGGATT CTGCGCGGGA CGTCCTTCTG CTACGTCCCT TCGGCCCTCA
ATCCAGCGGA CCTTCCTTCC CGCGGCCTGC TGCCGGCTCT GCGGCCTCTT CCGCGTCTTC GCCTTCGCCC TCAGACGAGT
CGGATCTCCC TTTGGGCCGC CTCCCCGCCT GGAATTCGAG CTCGGTACCT TTAAGACCAA TGACTTACAA GGCAGCTGTA
GATCTTAGCC ACTTTTTAAA AGAAAAGGGG GGACTGGAAG GGCTAATTCA CTCCCAACGA AGACAAGATC TGCTTTTTGC
TTGTACTGGG TCTCTCTGGT TAGACCAGAT CTGAGCCTGG GAGCTCTCTG GCTAACTAGG GAACCCACTG CTTAAGCCTC
AATAAAGCTT GCCTTGAGTG CTTCAAGTAG TGTGTGCCCG TCTGTTGTGT GACTCTGGTA ACTAGAGATC CCTCAGACCC
TTTTAGTCAG TGTGGAAAAT CTCTAGCAGT AGTAGTTCAT GTCATCTTAT TATTCAGTAT TTATAACTTG CAAAGAAATG
AATATCAGAG AGTGAGAGGA ACTTGTTTAT TGCAGCTTAT AATGGTTACA AATAAAGCAA TAGCATCACA AATTTCACAA
ATAAAGCATT TTTTTCACTG CATTCTAGTT GTGGTTTGTC CAAACTCATC AATGTATCTT ATCATGTCTG GCTCTAGCTA
TCCCGCCCCT AACTCCGCCC AGTTCCGCCC ATTCTCCGCC CCATGGCTGA CTAATTTTTT TTATTTATGC AGAGGCCGAG
GCCGCCTCGG CCTCTGAGCT ATTCCAGAAG TAGTGAGGAG GCTTTTTTGG AGGCCTAGGC TTTTGCGTCG AGACGTACCC
AATTCGCCCT ATAGTGAGTC GTATTACGCG CGCTCACTGG CCGTCGTTTT ACAACGTCGT GACTGGGAAA ACCCTGGCGT
TACCCAACTT AATCGCCTTG CAGCACATCC CCCTTTCGCC AGCTGGCGTA ATAGCGAAGA GGCCCGCACC GATCGCCCTT
CCCAACAGTT GCGCAGCCTG AATGGCGAAT GGCGCGACGC GCCCTGTAGC GGCGCATTAA GCGCGGCGGG TGTGGTGGTT
ACGCGCAGCG TGACCGCTAC ACTTGCCAGC GCCCTAGCGC CCGCTCCTTT CGCTTTCTTC CCTTCCTTTC TCGCCACGTT
CGCCGGCTTT CCCCGTCAAG CTCTAAATCG GGGGCTCCCT TTAGGGTTCC GATTTAGTGC TTTACGGCAC CTCGACCCCA
AAAAACTTGA TTAGGGTGAT GGTTCACGTA GTGGGCCATC GCCCTGATAG ACGGTTTTTC GCCCTTTGAC GTTGGAGTCC
ACGTTCTTTA ATAGTGGACT CTTGTTCCAA ACTGGAACAA CACTCAACCC TATCTCGGTC TATTCTTTTG ATTTATAAGG
GATTTTGCCG ATTTCGGCCT ATTGGTTAAA AAATGAGCTG ATTTAACAAA AATTTAACGC GAATTTTAAC AAAATATTAA
CGTTTACAAT TTCCCAGGTG GCACTTTTCG GGGAAATGTG CGCGGAACCC CTATTTGTTT ATTTTTCTAA ATACATTCAA
ATATGTATCC GCTCATGAGA CAATAACCCT GATAAATGCT TCAATAATAT TGAAAAAGGA AGAGTATGAG TATTCAACAT
TTCCGTGTCG CCCTTATTCC CTTTTTTGCG GCATTTTGCC TTCCTGTTTT TGCTCACCCA GAAACGCTGG TGAAAGTAAA
AGATGCTGAA GATCAGTTGG GTGCACGAGT GGGTTACATC GAACTGGATC TCAACAGCGG TAAGATCCTT GAGAGTTTTC
GCCCCGAAGA ACGTTTTCCA ATGATGAGCA CTTTTAAAGT TCTGCTATGT GGCGCGGTAT TATCCCGTAT TGACGCCGGG
CAAGAGCAAC TCGGTCGCCG CATACACTAT TCTCAGAATG ACTTGGTTGA GTACTCACCA GTCACAGAAA AGCATCTTAC
GGATGGCATG ACAGTAAGAG AATTATGCAG TGCTGCCATA ACCATGAGTG ATAACACTGC GGCCAACTTA CTTCTGACAA
CGATCGGAGG ACCGAAGGAG CTAACCGCTT TTTTGCACAA CATGGGGGAT CATGTAACTC GCCTTGATCG TTGGGAACCG
GAGCTGAATG AAGCCATACC AAACGACGAG CGTGACACCA CGATGCCTGT AGCAATGGCA ACAACGTTGC GCAAACTATT
AACTGGCGAA CTACTTACTC TAGCTTCCCG GCAACAATTA ATAGACTGGA TGGAGGCGGA TAAAGTTGCA GGACCACTTC
TGCGCTCGGC CCTTCCGGCT GGCTGGTTTA TTGCTGATAA ATCTGGAGCC GGTGAGCGTG GGTCTCGCGG TATCATTGCA
GCACTGGGGC CAGATGGTAA GCCCTCCCGT ATCGTAGTTA TCTACACGAC GGGGAGTCAG GCAACTATGG ATGAACGAAA
TAGACAGATC GCTGAGATAG GTGCCTCACT GATTAAGCAT TGGTAACTGT CAGACCAAGT TTACTCATAT ATACTTTAGA
TTGATTTAAA ACTTCATTTT TAATTTAAAA GGATCTAGGT GAAGATCCTT TTTGATAATC TCATGACCAA AATCCCTTAA
CGTGAGTTTT CGTTCCACTG AGCGTCAGAC CCCGTAGAAA AGATCAAAGG ATCTTCTTGA GATCCTTTTT TTCTGCGCGT
AATCTGCTGC TTGCAAACAA AAAAACCACC GCTACCAGCG GTGGTTTGTT TGCCGGATCA AGAGCTACCA ACTCTTTTTC
CGAAGGTAAC TGGCTTCAGC AGAGCGCAGA TACCAAATAC TGTCCTTCTA GTGTAGCCGT AGTTAGGCCA CCACTTCAAG
AACTCTGTAG CACCGCCTAC ATACCTCGCT CTGCTAATCC TGTTACCAGT GGCTGCTGCC AGTGGCGATA AGTCGTGTCT
TACCGGGTTG GACTCAAGAC GATAGTTACC GGATAAGGCG CAGCGGTCGG GCTGAACGGG GGGTTCGTGC ACACAGCCCA
GCTTGGAGCG AACGACCTAC ACCGAACTGA GATACCTACA GCGTGAGCTA TGAGAAAGCG CCACGCTTCC CGAAGGGAGA
AAGGCGGACA GGTATCCGGT AAGCGGCAGG GTCGGAACAG GAGAGCGCAC GAGGGAGCTT CCAGGGGGAA ACGCCTGGTA
TCTTTATAGT CCTGTCGGGT TTCGCCACCT CTGACTTGAG CGTCGATTTT TGTGATGCTC GTCAGGGGGG CGGAGCCTAT
GGAAAAACGC CAGCAACGCG GCCTTTTTAC GGTTCCTGGC CTTTTGCTGG CCTTTTGCTC ACATGTTCTT TCCTGCGTTA
TCCCCTGATT CTGTGGATAA CCGTATTACC GCCTTTGAGT GAGCTGATAC CGCTCGCCGC AGCCGAACGA CCGAGCGCAG
CGAGTCAGTG AGCGAGGAAG CGGAAGAGCG CCCAATACGC AAACCGCCTC TCCCCGCGCG TTGGCCGATT CATTAATGCA
GCTGGCACGA CAGGTTTCCC GACTGGAAAG CGGGCAGTGA GCGCAACGCA ATTAATGTGA GTTAGCTCAC TCATTAGGCA
CCCCAGGCTT TACACTTTAT GCTTCCGGCT CGTATGTTGT GTGGAATTGT GAGCGGATAA CAATTTCACA CAGGAAACAG
CTATGACCAT GATTACGCCA AGCGCGCAAT TAACCCTCAC TAAAGGGAAC AAAAGCTGGA GCTGCAAGCT TAATGTAGTC
TTATGCAATA CTCTTGTAGT CTTGCAACAT GGTAACGATG AGTTAGCAAC ATGCCTTACA AGGAGAGAAA AAGCACCGTG
CATGCCGATT GGTGGAAGTA AGGTGGTACG ATCGTGCCTT ATTAGGAAGG CAACAGACGG GTCTGACATG GATTGGACGA
ACCACTGAAT TGCCGCATTG CAGAGATATT GTATTTAAGT GCCTAGCTCG ATACAATAAA CGGGTCTCTC TGGTTAGACC
AGATCTGAGC CTGGGAGCTC TCTGGCTAAC TAGGGAACCC ACTGCTTAAG CCTCAATAAA GCTTGCCTTG AGTGCTTCAA
GTAGTGTGTG CCCGTCTGTT GTGTGACTCT GGTAACTAGA GATCCCTCAG ACCCTTTTAG TCAGTGTGGA AAATCTCTAG
CAGTGGCGCC CGAACAGGGA CCTGAAAGCG AAAGGGAAAC CAGAGCTCTC TCGACGCAGG ACTCGGCTTG CTGAAGCGCG
CACGGCAAGA GGCGAGGGGC GGCGACTGGT GAGTACGCCA AAAATTTTGA CTAGCGGAGG CTAGAAGGAG AGAGATGGGT
GCGAGAGCGT CAGTATTAAG CGGGGGAGAA TTAGATCGCG ATGGGAAAAA ATTCGGTTAA GGCCAGGGGG AAAGAAAAAA
TATAAATTAA AACATATAGT ATGGGCAAGC AGGGAGCTAG AACGATTCGC AGTTAATCCT GGCCTGTTAG AAACATCAGA
AGGCTGTAGA CAAATACTGG GACAGCTACA ACCATCCCTT CAGACAGGAT CAGAAGAACT TAGATCATTA TATAATACAG
TAGCAACCCT CTATTGTGTG CATCAAAGGA TAGAGATAAA AGACACCAAG GAAGCTTTAG ACAAGATAGA GGAAGAGCAA
AACAAAAGTA AGACCACCGC ACAGCAAGCG GCCGCTGATC TTCAGACCTG GAGGAGGAGA TATGAGGGAC AATTGGAGAA
GTGAATTATA TAAATATAAA GTAGTAAAAA TTGAACCATT AGGAGTAGCA CCCACCAAGG CAAAGAGAAG AGTGGTGCAG
AGAGAAAAAA GAGCAGTGGG AATAGGAGCT TTGTTCCTTG GGTTCTTGGG AGCAGCAGGA AGCACTATGG GCGCAGCCTC
AATGACGCTG ACGGTACAGG CCAGACAATT ATTGTCTGGT ATAGTGCAGC AGCAGAACAA TTTGCTGAGG GCTATTGAGG
CGCAACAGCA TCTGTTGCAA CTCACAGTCT GGGGCATCAA GCAGCTCCAG GCAAGAATCC TGGCTGTGGA AAGATACCTA
AAGGATCAAC AGCTCCTGGG GATTTGGGGT TGCTCTGGAA AACTCATTTG CACCACTGCT GTGCCTTGGA ATGCTAGTTG
GAGTAATAAA TCTCTGGAAC AGATTGGAAT CACACGACCT GGATGGAGTG GGACAGAGAA ATTAACAATT ACACAAGCTT
AATACACTCC TTAATTGAAG AATCGCAAAA CCAGCAAGAA AAGAATGAAC AAGAATTATT GGAATTAGAT AAATGGGCAA
GTTTGTGGAA TTGGTTTAAC ATAACAAATT GGCTGTGGTA TATAAAATTA TTCATAATGA TAGTAGGAGG CTTGGTAGGT
TTAAGAATAG TTTTTGCTGT ACTTTCTATA GTGAATAGAG TTAGGCAGGG ATATTCACCA TTATCGTTTC AGACCCACCT
CCCAACCCCG AGGGGACCCG ACAGGCCCGA AGGAATAGAA GAAGAAGGTG GAGAGAGAGA CAGAGACAGA TCCATTCGAT
TAGTGAACGG ATCTCGACGG TATCGATTAG ACTGTAGCCC AGGAATATGG CAGCTAGATT GTACACATTT AGAAGGAAAA
GTTATCTTGG TAGCAGTTCA TGTAGCCAGT GGATATATAG AAGCAGAAGT AATTCCAGCA GAGACAGGGC AAGAAACAGC
ATACTTCCTC TTAAAATTAG CAGGAAGATG GCCAGTAAAA ACAGTACATA CAGACAATGG CAGCAATTTC ACCAGTACTA
CAGTTAAGGC CGCCTGTTGG TGGGCGGGGA TCAAGCAGGA ATTTGGCATT CCCTACAATC CCCAAAGTCA AGGAGTAATA
GAATCTATGA ATAAAGAATT AAAGAAAATT ATAGGACAGG TAAGAGATCA GGCTGAACAT CTTAAGACAG CAGTACAAAT
GGCAGTATTC ATCCACAATT TTAAAAGAAA AGGGGGGATT GGGGGGTACA GTGCAGGGGA AAGAATAGTA GACATAATAG
CAACAGACAT ACAAACTAAA GAATTACAAA AACAAATTAC AAAAATTCAA AATTTTCGGG TTTATTACAG GGACAGCAGA
GATCCAGTTT GGCTGCATTG ATCACGTGAG GCTCCGGTGC CCGTCAGTGG GCAGAGCGCA CATCGCCCAC AGTCCCCGAG
AAGTTGGGGG GAGGGGTCGG CAATTGAACC GGTGCCTAGA GAAGGTGGCG CGGGGTAAAC TGGGAAAGTG ATGTCGTGTA
CTGGCTCCGC CTTTTTCCCG AGGGTGGGGG AGAACCGTAT ATAAGTGCAG TAGTCGCCGT GAACGTTCTT TTTCGCAACG
GGTTTGCCGC CAGAACACAG GTAAGTGCCG TGTGTGGTTC CCGCGGGCCT GGCCTCTTTA CGGGTTATGG CCCTTGCGTG
CCTTGAATTA CTTCCACCTG GCTGCAGTAC GTGATTCTTG ATCCCGAGCT TCGGGTTGGA AGTGGGTGGG AGAGTTCGAG
GCCTTGCGCT TAAGGAGCCC CTTCGCCTCG TGCTTGAGTT GAGGCCTGGC CTGGGCGCTG GGGCCGCCGC GTGCGAATCT
GGTGGCACCT TCGCGCCTGT CTCGCTGCTT TCGATAAGTC TCTAGCCATT TAAAATTTTT GATGACCTGC TGCGACGCTT
TTTTTCTGGC AAGATAGTCT TGTAAATGCG GGCCAAGATC TGCACACTGG TATTTCGGTT TTTGGGGCCG CGGGCGGCGA
CGGGGCCCGT GCGTCCCAGC GCACATGTTC GGCGAGGCGG GGCCTGCGAG CGCGGCCACC GAGAATCGGA CGGGGGTAGT
CTCAAGCTGG CCGGCCTGCT CTGGTGCCTG GCCTCGCGCC GCCGTGTATC GCCCCGCCCT GGGCGGCAAG GCTGGCCCGG
TCGGCACCAG TTGCGTGAGC GGAAAGATGG CCGCTTCCCG GCCCTGCTGC AGGGAGCTCA AAATGGAGGA CGCGGCGCTC
GGGAGAGCGG GCGGGTGAGT CACCCACACA AAGGAAAAGG GCCTTTCCGT CCTCAGCCGT CGCTTCATGT GACTCCACGG
AGTACCGGGC GCCGTCCAGG CACCTCGATT AGTTCTCGAG CTTTTGGAGT ACGTCGTCTT TAGGTTGGGG GGAGGGGTTT
TATGCGATGG AGTTTCCCCA CACTGAGTGG GTGGAGACTG AAGTTAGGCC AGCTTGGCAC TTGATGTAAT TCTCCTTGGA
ATTTGCCCTT TTTGAGTTTG GATCTTGGTT CATTCTCAAG CCTCAGACAG TGGTTCAAAG TTTTTTTCTT CCATTTCAGG
TGTCGTGATC TAGAG
hFVIII-A2-BBz-T2A-mCherry
(SEQ ID NO: 14)
MEFGLSWLFL VAILKGVQCG SSVAKKHPKT WVHYIAAEEE DWDYAPLVLA PDDRSYKSQY LNNGPQRIGR KYKKVRFMAY
TDETFKTREA IQHESGILGP LLYGEVGDTL LIIFKNQASR PYNIYPHGIT DVRPLYSRRL PKGVKHLKDF PILPGEIFKY
KWTVTVEDGP TKSDPRCLTR YYSSFVNMER DLASGLIGPL LICYKESVDQ RGNQIMSDKR NVILFSVFDE NRSWYLTENI
QRFLPNPAGV QLEDPEFQAS NIMHSINGYV FDSLQLSVCL HEVAYWYILS IGAQTDFLSV FFSGYTFKHK MVYEDTLTLF
PFSGETVFMS MENPGLWILG CHNSDFRNRG MTALLKVSSC DKNTGDYYED SYEDISAYLL SKNNAIEPRA STTTPAPRPP
TPAPTIASQP LSLRPEACRP AAGGAVHTRG LDFACDSGIY IWAPLAGTCG VLLLSLVITL YCKRGRKKLL YIFKQPFMRP
VQTTQEEDGC SCRFPEEEEG GCELRVKFSR SADAPAYQQG QNQLYNELNL GRREEYDVLD KRRGRDPEMG GKPRRKNPQE
GLYNELQKDK MAEAYSEIGM KGERRRGKGH DGLYQGLSTA TKDTYDALHM QALPPRGSGE GRGSLLTCGD VEENPGPTRM
VSKGEEDNMA IIKEFMRFKV HMEGSVNGHE FEIEGEGEGR PYEGTQTAKL KVTKGGPLPF AWDILSPQFM YGSKAYVKHP
ADIPDYLKLS FPEGFKWERV MNFEDGGVVT VTQDSSLQDG EFIYKVKLRG TNFPSDGPVM QKKTMGWEAS SERMYPEDGA
LKGEIKQRLK LKDGGHYDAE VKTTYKAKKP VQLPGAYNVN IKLDITSHNE DYTIVEQYER AEGRHSTGGM DELYK
hFVIII-A2-BBz-T2A
(SEQ ID NO: 15)
MEFGLSWLFL VAILKGVQCG SSVAKKHPKT WVHYIAAEEE DWDYAPLVLA PDDRSYKSQY LNNGPQRIGR KYKKVRFMAY
TDETFKTREA IQHESGILGP LLYGEVGDTL LIIFKNQASR PYNIYPHGIT DVRPLYSRRL PKGVKHLKDF PILPGEIFKY
KWTVTVEDGP TKSDPRCLTR YYSSFVNMER DLASGLIGPL LICYKESVDQ RGNQIMSDKR NVILFSVFDE NRSWYLTENI
QRFLPNPAGV QLEDPEFQAS NIMHSINGYV FDSLQLSVCL HEVAYWYILS IGAQTDFLSV FFSGYTFKHK MVYEDTLTLF
PFSGETVFMS MENPGLWILG CHNSDFRNRG MTALLKVSSC DKNTGDYYED SYEDISAYLL SKNNAIEPRA STTTPAPRPP
TPAPTIASQP LSLRPEACRP AAGGAVHTRG LDFACDSGIY IWAPLAGTCG VLLLSLVITL YCKRGRKKLL YIFKQPFMRP
VQTTQEEDGC SCRFPEEEEG GCELRVKFSR SADAPAYQQG QNQLYNELNL GRREEYDVLD KRRGRDPEMG GKPRRKNPQE
GLYNELQKDK MAEAYSEIGM KGERRRGKGH DGLYQGLSTA TKDTYDALHM QALPPR
pELPS-hFVIII-C2-BBz-T2A-mCherry
(SEQ ID NO: 16)
GATCTATGGA GTTTGGGCTG AGCTGGCTTT TTCTTGTGGC TATTTTAAAA GGTGTCCAGT GCGGATCCAA TAGTTGCAGC
ATGCCATTGG GAATGGAGAG TAAAGCAATA TCAGATGCAC AGATTACTGC TTCATCCTAC TTTACCAATA TGTTTGCCAC
CTGGTCTCCT TCAAAAGCTC GACTTCACCT CCAAGGGAGG AGTAATGCCT GGAGACCTCA GGTGAATAAT CCAAAAGAGT
GGCTGCAAGT GGACTTCCAG AAGACAATGA AAGTCACAGG AGTAACTACT CAGGGAGTAA AATCTCTGCT TACCAGCATG
TATGTGAAGG AGTTCCTCAT CTCCAGCAGT CAAGATGGCC ATCAGTGGAC TCTCTTTTTT CAGAATGGCA AAGTAAAGGT
TTTTCAGGGA AATCAAGACT CCTTCACACC TGTGGTGAAC TCTCTAGACC CACCGTTACT GACTCGCTAC CTTCGAATTC
ACCCCCAGAG TTGGGTGCAC CAGATTGCCC TGAGGATGGA GGTTCTGGGC TGCGAGGCAC AGGACCTCTA CGCTAGCACC
ACGACGCCAG CGCCGCGACC ACCAACACCG GCGCCCACCA TCGCGTCGCA GCCCCTGTCC CTGCGCCCAG AGGCGTGCCG
GCCAGCGGCG GGGGGCGCAG TGCACACGAG GGGGCTGGAC TTCGCCTGTG ATTCCGGAAT CTACATCTGG GCCCCTCTGG
CCGGCACCTG TGGCGTGCTG CTGCTGTCCC TGGTCATCAC CCTGTACTGC AAGCGGGGCA GAAAGAAGCT GCTGTACATC
TTCAAGCAGC CCTTCATGCG GCCTGTGCAG ACCACACAGG AAGAGGACGG CTGTAGCTGT AGATTCCCCG AGGAAGAGGA
AGGCGGCTGC GAGCTGAGAG TGAAGTTCAG CAGAAGCGCC GACGCCCCTG CCTATCAGCA GGGCCAGAAC CAGCTGTACA
ACGAGCTGAA CCTGGGCAGA CGGGAGGAAT ACGACGTGCT GGACAAGAGA AGAGGCCGGG ACCCTGAGAT GGGCGGCAAG
CCCAGACGGA AGAACCCCCA GGAAGGCCTG TATAACGAAC TGCAGAAAGA CAAGATGGCC GAGGCCTACA GCGAGATCGG
CATGAAGGGC GAGCGGAGAA GAGGCAAGGG CCATGACGGC CTGTACCAGG GCCTGAGCAC CGCCACCAAG GACACCTACG
ACGCCCTGCA CATGCAGGCC CTGCCTCCAA GAGGCAGCGG AGAGGGCAGA GGAAGTCTTC TAACATGCGG TGACGTGGAG
GAGAATCCCG GCCCTACGCG TATGGTGAGC AAGGGCGAGG AGGATAACAT GGCCATCATC AAGGAGTTCA TGCGCTTCAA
GGTGCACATG GAGGGCTCCG TGAACGGCCA CGAGTTCGAG ATCGAGGGCG AGGGCGAGGG CCGCCCCTAC GAGGGCACCC
AGACCGCCAA GCTGAAGGTG ACCAAGGGTG GCCCCCTGCC CTTCGCCTGG GACATCCTGT CCCCTCAGTT CATGTACGGC
TCCAAGGCCT ACGTGAAGCA CCCCGCCGAC ATCCCCGACT ACTTGAAGCT GTCCTTCCCC GAGGGCTTCA AGTGGGAGCG
CGTGATGAAC TTCGAGGACG GCGGCGTGGT GACCGTGACC CAGGACTCCT CCCTGCAGGA CGGCGAGTTC ATCTACAAGG
TGAAGCTGCG CGGCACCAAC TTCCCCTCCG ACGGCCCCGT AATGCAGAAG AAGACCATGG GCTGGGAGGC CTCCTCCGAG
CGGATGTACC CCGAGGACGG CGCCCTGAAG GGCGAGATCA AGCAGAGGCT GAAGCTGAAG GACGGCGGCC ACTACGACGC
TGAGGTCAAG ACCACCTACA AGGCCAAGAA GCCCGTGCAG CTGCCCGGCG CCTACAACGT CAACATCAAG TTGGACATCA
CCTCCCACAA CGAGGACTAC ACCATCGTGG AACAGTACGA ACGCGCCGAG GGCCGCCACT CCACCGGCGG CATGGACGAG
CTGTACAAGT AGGTCGACAA TCAACCTCTG GATTACAAAA TTTGTGAAAG ATTGACTGGT ATTCTTAACT ATGTTGCTCC
TTTTACGCTA TGTGGATACG CTGCTTTAAT GCCTTTGTAT CATGCTATTG CTTCCCGTAT GGCTTTCATT TTCTCCTCCT
TGTATAAATC CTGGTTGCTG TCTCTTTATG AGGAGTTGTG GCCCGTTGTC AGGCAACGTG GCGTGGTGTG CACTGTGTTT
GCTGACGCAA CCCCCACTGG TTGGGGCATT GCCACCACCT GTCAGCTCCT TTCCGGGACT TTCGCTTTCC CCCTCCCTAT
TGCCACGGCG GAACTCATCG CCGCCTGCCT TGCCCGCTGC TGGACAGGGG CTCGGCTGTT GGGCACTGAC AATTCCGTGG
TGTTGTCGGG GAAGCTGACG TCCTTTCCAT GGCTGCTCGC CTGTGTTGCC ACCTGGATTC TGCGCGGGAC GTCCTTCTGC
TACGTCCCTT CGGCCCTCAA TCCAGCGGAC CTTCCTTCCC GCGGCCTGCT GCCGGCTCTG CGGCCTCTTC CGCGTCTTCG
CCTTCGCCCT CAGACGAGTC GGATCTCCCT TTGGGCCGCC TCCCCGCCTG GAATTCGAGC TCGGTACCTT TAAGACCAAT
GACTTACAAG GCAGCTGTAG ATCTTAGCCA CTTTTTAAAA GAAAAGGGGG GACTGGAAGG GCTAATTCAC TCCCAACGAA
GACAAGATCT GCTTTTTGCT TGTACTGGGT CTCTCTGGTT AGACCAGATC TGAGCCTGGG AGCTCTCTGG CTAACTAGGG
AACCCACTGC TTAAGCCTCA ATAAAGCTTG CCTTGAGTGC TTCAAGTAGT GTGTGCCCGT CTGTTGTGTG ACTCTGGTAA
CTAGAGATCC CTCAGACCCT TTTAGTCAGT GTGGAAAATC TCTAGCAGTA GTAGTTCATG TCATCTTATT ATTCAGTATT
TATAACTTGC AAAGAAATGA ATATCAGAGA GTGAGAGGAA CTTGTTTATT GCAGCTTATA ATGGTTACAA ATAAAGCAAT
AGCATCACAA ATTTCACAAA TAAAGCATTT TTTTCACTGC ATTCTAGTTG TGGTTTGTCC AAACTCATCA ATGTATCTTA
TCATGTCTGG CTCTAGCTAT CCCGCCCCTA ACTCCGCCCA GTTCCGCCCA TTCTCCGCCC CATGGCTGAC TAATTTTTTT
TATTTATGCA GAGGCCGAGG CCGCCTCGGC CTCTGAGCTA TTCCAGAAGT AGTGAGGAGG CTTTTTTGGA GGCCTAGGCT
TTTGCGTCGA GACGTACCCA ATTCGCCCTA TAGTGAGTCG TATTACGCGC GCTCACTGGC CGTCGTTTTA CAACGTCGTG
ACTGGGAAAA CCCTGGCGTT ACCCAACTTA ATCGCCTTGC AGCACATCCC CCTTTCGCCA GCTGGCGTAA TAGCGAAGAG
GCCCGCACCG ATCGCCCTTC CCAACAGTTG CGCAGCCTGA ATGGCGAATG GCGCGACGCG CCCTGTAGCG GCGCATTAAG
CGCGGCGGGT GTGGTGGTTA CGCGCAGCGT GACCGCTACA CTTGCCAGCG CCCTAGCGCC CGCTCCTTTC GCTTTCTTCC
CTTCCTTTCT CGCCACGTTC GCCGGCTTTC CCCGTCAAGC TCTAAATCGG GGGCTCCCTT TAGGGTTCCG ATTTAGTGCT
TTACGGCACC TCGACCCCAA AAAACTTGAT TAGGGTGATG GTTCACGTAG TGGGCCATCG CCCTGATAGA CGGTTTTTCG
CCCTTTGACG TTGGAGTCCA CGTTCTTTAA TAGTGGACTC TTGTTCCAAA CTGGAACAAC ACTCAACCCT ATCTCGGTCT
ATTCTTTTGA TTTATAAGGG ATTTTGCCGA TTTCGGCCTA TTGGTTAAAA AATGAGCTGA TTTAACAAAA ATTTAACGCG
AATTTTAACA AAATATTAAC GTTTACAATT TCCCAGGTGG CACTTTTCGG GGAAATGTGC GCGGAACCCC TATTTGTTTA
TTTTTCTAAA TACATTCAAA TATGTATCCG CTCATGAGAC AATAACCCTG ATAAATGCTT CAATAATATT GAAAAAGGAA
GAGTATGAGT ATTCAACATT TCCGTGTCGC CCTTATTCCC TTTTTTGCGG CATTTTGCCT TCCTGTTTTT GCTCACCCAG
AAACGCTGGT GAAAGTAAAA GATGCTGAAG ATCAGTTGGG TGCACGAGTG GGTTACATCG AACTGGATCT CAACAGCGGT
AAGATCCTTG AGAGTTTTCG CCCCGAAGAA CGTTTTCCAA TGATGAGCAC TTTTAAAGTT CTGCTATGTG GCGCGGTATT
ATCCCGTATT GACGCCGGGC AAGAGCAACT CGGTCGCCGC ATACACTATT CTCAGAATGA CTTGGTTGAG TACTCACCAG
TCACAGAAAA GCATCTTACG GATGGCATGA CAGTAAGAGA ATTATGCAGT GCTGCCATAA CCATGAGTGA TAACACTGCG
GCCAACTTAC TTCTGACAAC GATCGGAGGA CCGAAGGAGC TAACCGCTTT TTTGCACAAC ATGGGGGATC ATGTAACTCG
CCTTGATCGT TGGGAACCGG AGCTGAATGA AGCCATACCA AACGACGAGC GTGACACCAC GATGCCTGTA GCAATGGCAA
CAACGTTGCG CAAACTATTA ACTGGCGAAC TACTTACTCT AGCTTCCCGG CAACAATTAA TAGACTGGAT GGAGGCGGAT
AAAGTTGCAG GACCACTTCT GCGCTCGGCC CTTCCGGCTG GCTGGTTTAT TGCTGATAAA TCTGGAGCCG GTGAGCGTGG
GTCTCGCGGT ATCATTGCAG CACTGGGGCC AGATGGTAAG CCCTCCCGTA TCGTAGTTAT CTACACGACG GGGAGTCAGG
CAACTATGGA TGAACGAAAT AGACAGATCG CTGAGATAGG TGCCTCACTG ATTAAGCATT GGTAACTGTC AGACCAAGTT
TACTCATATA TACTTTAGAT TGATTTAAAA CTTCATTTTT AATTTAAAAG GATCTAGGTG AAGATCCTTT TTGATAATCT
CATGACCAAA ATCCCTTAAC GTGAGTTTTC GTTCCACTGA GCGTCAGACC CCGTAGAAAA GATCAAAGGA TCTTCTTGAG
ATCCTTTTTT TCTGCGCGTA ATCTGCTGCT TGCAAACAAA AAAACCACCG CTACCAGCGG TGGTTTGTTT GCCGGATCAA
GAGCTACCAA CTCTTTTTCC GAAGGTAACT GGCTTCAGCA GAGCGCAGAT ACCAAATACT GTCCTTCTAG TGTAGCCGTA
GTTAGGCCAC CACTTCAAGA ACTCTGTAGC ACCGCCTACA TACCTCGCTC TGCTAATCCT GTTACCAGTG GCTGCTGCCA
GTGGCGATAA GTCGTGTCTT ACCGGGTTGG ACTCAAGACG ATAGTTACCG GATAAGGCGC AGCGGTCGGG CTGAACGGGG
GGTTCGTGCA CACAGCCCAG CTTGGAGCGA ACGACCTACA CCGAACTGAG ATACCTACAG CGTGAGCTAT GAGAAAGCGC
CACGCTTCCC GAAGGGAGAA AGGCGGACAG GTATCCGGTA AGCGGCAGGG TCGGAACAGG AGAGCGCACG AGGGAGCTTC
CAGGGGGAAA CGCCTGGTAT CTTTATAGTC CTGTCGGGTT TCGCCACCTC TGACTTGAGC GTCGATTTTT GTGATGCTCG
TCAGGGGGGC GGAGCCTATG GAAAAACGCC AGCAACGCGG CCTTTTTACG GTTCCTGGCC TTTTGCTGGC CTTTTGCTCA
CATGTTCTTT CCTGCGTTAT CCCCTGATTC TGTGGATAAC CGTATTACCG CCTTTGAGTG AGCTGATACC GCTCGCCGCA
GCCGAACGAC CGAGCGCAGC GAGTCAGTGA GCGAGGAAGC GGAAGAGCGC CCAATACGCA AACCGCCTCT CCCCGCGCGT
TGGCCGATTC ATTAATGCAG CTGGCACGAC AGGTTTCCCG ACTGGAAAGC GGGCAGTGAG CGCAACGCAA TTAATGTGAG
TTAGCTCACT CATTAGGCAC CCCAGGCTTT ACACTTTATG CTTCCGGCTC GTATGTTGTG TGGAATTGTG AGCGGATAAC
AATTTCACAC AGGAAACAGC TATGACCATG ATTACGCCAA GCGCGCAATT AACCCTCACT AAAGGGAACA AAAGCTGGAG
CTGCAAGCTT AATGTAGTCT TATGCAATAC TCTTGTAGTC TTGCAACATG GTAACGATGA GTTAGCAACA TGCCTTACAA
GGAGAGAAAA AGCACCGTGC ATGCCGATTG GTGGAAGTAA GGTGGTACGA TCGTGCCTTA TTAGGAAGGC AACAGACGGG
TCTGACATGG ATTGGACGAA CCACTGAATT GCCGCATTGC AGAGATATTG TATTTAAGTG CCTAGCTCGA TACAATAAAC
GGGTCTCTCT GGTTAGACCA GATCTGAGCC TGGGAGCTCT CTGGCTAACT AGGGAACCCA CTGCTTAAGC CTCAATAAAG
CTTGCCTTGA GTGCTTCAAG TAGTGTGTGC CCGTCTGTTG TGTGACTCTG GTAACTAGAG ATCCCTCAGA CCCTTTTAGT
CAGTGTGGAA AATCTCTAGC AGTGGCGCCC GAACAGGGAC CTGAAAGCGA AAGGGAAACC AGAGCTCTCT CGACGCAGGA
CTCGGCTTGC TGAAGCGCGC ACGGCAAGAG GCGAGGGGCG GCGACTGGTG AGTACGCCAA AAATTTTGAC TAGCGGAGGC
TAGAAGGAGA GAGATGGGTG CGAGAGCGTC AGTATTAAGC GGGGGAGAAT TAGATCGCGA TGGGAAAAAA TTCGGTTAAG
GCCAGGGGGA AAGAAAAAAT ATAAATTAAA ACATATAGTA TGGGCAAGCA GGGAGCTAGA ACGATTCGCA GTTAATCCTG
GCCTGTTAGA AACATCAGAA GGCTGTAGAC AAATACTGGG ACAGCTACAA CCATCCCTTC AGACAGGATC AGAAGAACTT
AGATCATTAT ATAATACAGT AGCAACCCTC TATTGTGTGC ATCAAAGGAT AGAGATAAAA GACACCAAGG AAGCTTTAGA
CAAGATAGAG GAAGAGCAAA ACAAAAGTAA GACCACCGCA CAGCAAGCGG CCGCTGATCT TCAGACCTGG AGGAGGAGAT
ATGAGGGACA ATTGGAGAAG TGAATTATAT AAATATAAAG TAGTAAAAAT TGAACCATTA GGAGTAGCAC CCACCAAGGC
AAAGAGAAGA GTGGTGCAGA GAGAAAAAAG AGCAGTGGGA ATAGGAGCTT TGTTCCTTGG GTTCTTGGGA GCAGCAGGAA
GCACTATGGG CGCAGCCTCA ATGACGCTGA CGGTACAGGC CAGACAATTA TTGTCTGGTA TAGTGCAGCA GCAGAACAAT
TTGCTGAGGG CTATTGAGGC GCAACAGCAT CTGTTGCAAC TCACAGTCTG GGGCATCAAG CAGCTCCAGG CAAGAATCCT
GGCTGTGGAA AGATACCTAA AGGATCAACA GCTCCTGGGG ATTTGGGGTT GCTCTGGAAA ACTCATTTGC ACCACTGCTG
TGCCTTGGAA TGCTAGTTGG AGTAATAAAT CTCTGGAACA GATTGGAATC ACACGACCTG GATGGAGTGG GACAGAGAAA
TTAACAATTA CACAAGCTTA ATACACTCCT TAATTGAAGA ATCGCAAAAC CAGCAAGAAA AGAATGAACA AGAATTATTG
GAATTAGATA AATGGGCAAG TTTGTGGAAT TGGTTTAACA TAACAAATTG GCTGTGGTAT ATAAAATTAT TCATAATGAT
AGTAGGAGGC TTGGTAGGTT TAAGAATAGT TTTTGCTGTA CTTTCTATAG TGAATAGAGT TAGGCAGGGA TATTCACCAT
TATCGTTTCA GACCCACCTC CCAACCCCGA GGGGACCCGA CAGGCCCGAA GGAATAGAAG AAGAAGGTGG AGAGAGAGAC
AGAGACAGAT CCATTCGATT AGTGAACGGA TCTCGACGGT ATCGATTAGA CTGTAGCCCA GGAATATGGC AGCTAGATTG
TACACATTTA GAAGGAAAAG TTATCTTGGT AGCAGTTCAT GTAGCCAGTG GATATATAGA AGCAGAAGTA ATTCCAGCAG
AGACAGGGCA AGAAACAGCA TACTTCCTCT TAAAATTAGC AGGAAGATGG CCAGTAAAAA CAGTACATAC AGACAATGGC
AGCAATTTCA CCAGTACTAC AGTTAAGGCC GCCTGTTGGT GGGCGGGGAT CAAGCAGGAA TTTGGCATTC CCTACAATCC
CCAAAGTCAA GGAGTAATAG AATCTATGAA TAAAGAATTA AAGAAAATTA TAGGACAGGT AAGAGATCAG GCTGAACATC
TTAAGACAGC AGTACAAATG GCAGTATTCA TCCACAATTT TAAAAGAAAA GGGGGGATTG GGGGGTACAG TGCAGGGGAA
AGAATAGTAG ACATAATAGC AACAGACATA CAAACTAAAG AATTACAAAA ACAAATTACA AAAATTCAAA ATTTTCGGGT
TTATTACAGG GACAGCAGAG ATCCAGTTTG GCTGCATTGA TCACGTGAGG CTCCGGTGCC CGTCAGTGGG CAGAGCGCAC
ATCGCCCACA GTCCCCGAGA AGTTGGGGGG AGGGGTCGGC AATTGAACCG GTGCCTAGAG AAGGTGGCGC GGGGTAAACT
GGGAAAGTGA TGTCGTGTAC TGGCTCCGCC TTTTTCCCGA GGGTGGGGGA GAACCGTATA TAAGTGCAGT AGTCGCCGTG
AACGTTCTTT TTCGCAACGG GTTTGCCGCC AGAACACAGG TAAGTGCCGT GTGTGGTTCC CGCGGGCCTG GCCTCTTTAC
GGGTTATGGC CCTTGCGTGC CTTGAATTAC TTCCACCTGG CTGCAGTACG TGATTCTTGA TCCCGAGCTT CGGGTTGGAA
GTGGGTGGGA GAGTTCGAGG CCTTGCGCTT AAGGAGCCCC TTCGCCTCGT GCTTGAGTTG AGGCCTGGCC TGGGCGCTGG
GGCCGCCGCG TGCGAATCTG GTGGCACCTT CGCGCCTGTC TCGCTGCTTT CGATAAGTCT CTAGCCATTT AAAATTTTTG
ATGACCTGCT GCGACGCTTT TTTTCTGGCA AGATAGTCTT GTAAATGCGG GCCAAGATCT GCACACTGGT ATTTCGGTTT
TTGGGGCCGC GGGCGGCGAC GGGGCCCGTG CGTCCCAGCG CACATGTTCG GCGAGGCGGG GCCTGCGAGC GCGGCCACCG
AGAATCGGAC GGGGGTAGTC TCAAGCTGGC CGGCCTGCTC TGGTGCCTGG CCTCGCGCCG CCGTGTATCG CCCCGCCCTG
GGCGGCAAGG CTGGCCCGGT CGGCACCAGT TGCGTGAGCG GAAAGATGGC CGCTTCCCGG CCCTGCTGCA GGGAGCTCAA
AATGGAGGAC GCGGCGCTCG GGAGAGCGGG CGGGTGAGTC ACCCACACAA AGGAAAAGGG CCTTTCCGTC CTCAGCCGTC
GCTTCATGTG ACTCCACGGA GTACCGGGCG CCGTCCAGGC ACCTCGATTA GTTCTCGAGC TTTTGGAGTA CGTCGTCTTT
AGGTTGGGGG GAGGGGTTTT ATGCGATGGA GTTTCCCCAC ACTGAGTGGG TGGAGACTGA AGTTAGGCCA GCTTGGCACT
TGATGTAATT CTCCTTGGAA TTTGCCCTTT TTGAGTTTGG ATCTTGGTTC ATTCTCAAGC CTCAGACAGT GGTTCAAAGT
TTTTTTCTTC CATTTCAGGT GTCGTGATCT AGAG
pELPS-hFVIII-C2-BBz-T2A-mCherry
(SEQ ID NO: 17)
MEFGLSWLFL VAILKGVQCG SNSCSMPLGM ESKAISDAQI TASSYFTNMF ATWSPSKARL HLQGRSNAWR PQVNNPKEWL
QVDFQKTMKV TGVTTQGVKS LLTSMYVKEF LISSSQDGHQ WTLFFQNGKV KVFQGNQDSF TPVVNSLDPP LLTRYLRIHP
QSWVHQIALR MEVLGCEAQD LYASTTTPAP RPPTPAPTIA SQPLSLRPEA CRPAAGGAVH TRGLDFACDS GIYIWAPLAG
TCGVLLLSLV ITLYCKRGRK KLLYIFKQPF MRPVQTTQEE DGCSCRFPEE EEGGCELRVK FSRSADAPAY QQGQNQLYNE
LNLGRREEYD VLDKRRGRDP EMGGKPRRKN PQEGLYNELQ KDKMAEAYSE IGMKGERRRG KGHDGLYQGL STATKDTYDA
LHMQALPPRG SGEGRGSLLT CGDVEENPGP TRMVSKGEED NMAIIKEFMR FKVHMEGSVN GHEFEIEGEG EGRPYEGTQT
AKLKVTKGGP LPFAWDILSP QFMYGSKAYV KHPADIPDYL KLSFPEGFKW ERVMNFEDGG VVTVTQDSSL QDGEFIYKVK
LRGTNFPSDG PVMQKKTMGW EASSERMYPE DGALKGEIKQ RLKLKDGGHY DAEVKTTYKA KKPVQLPGAY NVNIKLDITS
HNEDYTIVEQ YERAEGRHST GGMDELYK
hFVIII-C2-BBz
(SEQ ID NO: 18)
MEFGLSWLFL VAILKGVQCG SNSCSMPLGM ESKAISDAQI TASSYFTNMF ATWSPSKARL HLQGRSNAWR PQVNNPKEWL
QVDFQKTMKV TGVTTQGVKS LLTSMYVKEF LISSSQDGHQ WTLFFQNGKV KVFQGNQDSF TPVVNSLDPP LLTRYLRIHP
QSWVHQIALR MEVLGCEAQD LYASTTTPAP RPPTPAPTIA SQPLSLRPEA CRPAAGGAVH TRGLDFACDS GIYIWAPLAG
TCGVLLLSLV ITLYCKRGRK KLLYIFKQPF MRPVQTTQEE DGCSCRFPEE EEGGCELRVK FSRSADAPAY QQGQNQLYNE
LNLGRREEYD VLDKRRGRDP EMGGKPRRKN PQEGLYNELQ KDKMAEAYSE IGMKGERRRG KGHDGLYQGL STATKDTYDA
LHMQALPPR
pTRPE-hFVIII-A2-BBz
(SEQ ID NO: 19)
GTGCACGAGT GGGTTACATC GAACTGGATC TCAACAGCGG TAAGATCCTT GAGAGTTTTC GCCCCGAAGA ACGTTTTCCA
ATGATGAGCA CTTTTAAAGT TCTGCTATGT GGCGCGGTAT TATCCCGTAT TGACGCCGGG CAAGAGCAAC TCGGTCGCCG
CATACACTAT TCTCAGAATG ACTTGGTTGA GTACTCACCA GTCACAGAAA AGCATCTTAC GGATGGCATG ACAGTAAGAG
AATTATGCAG TGCTGCCATA ACCATGAGTG ATAACACTGC GGCCAACTTA CTTCTGACAA CGATCGGAGG ACCGAAGGAG
CTAACCGCTT TTTTGCACAA CATGGGGGAT CATGTAACTC GCCTTGATCG TTGGGAACCG GAGCTGAATG AAGCCATACC
AAACGACGAG CGTGACACCA CGATGCCTGT AGCAATGGCA ACAACGTTGC GCAAACTATT AACTGGCGAA CTACTTACTC
TAGCTTCCCG GCAACAATTA ATAGACTGGA TGGAGGCGGA TAAAGTTGCA GGACCACTTC TGCGCTCGGC CCTTCCGGCT
GGCTGGTTTA TTGCTGATAA ATCTGGAGCC GGTGAGCGTG GGTCTCGCGG TATCATTGCA GCACTGGGGC CAGATGGTAA
GCCCTCCCGT ATCGTAGTTA TCTACACGAC GGGGAGTCAG GCAACTATGG ATGAACGAAA TAGACAGATC GCTGAGATAG
GTGCCTCACT GATTAAGCAT TGGTAACTGT CAGACCAAGT TTACTCATAT ATACTTTAGA TTGATTTAAA ACTTCATTTT
TAATTTAAAA GGATCTAGGT GAAGATCCTT TTTGATAATC TCATGACCAA AATCCCTTAA CGTGAGTTTT CGTTCCACTG
AGCGTCAGAC CCCGTAGAAA AGATCAAAGG ATCTTCTTGA GATCCTTTTT TTCTGCGCGT AATCTGCTGC TTGCAAACAA
AAAAACCACC GCTACCAGCG GTGGTTTGTT TGCCGGATCA AGAGCTACCA ACTCTTTTTC CGAAGGTAAC TGGCTTCAGC
AGAGCGCAGA TACCAAATAC TGTTCTTCTA GTGTAGCCGT AGTTAGGCCA CCACTTCAAG AACTCTGTAG CACCGCCTAC
ATACCTCGCT CTGCTAATCC TGTTACCAGT GGCTGCTGCC AGTGGCGATA AGTCGTGTCT TACCGGGTTG GACTCAAGAC
GATAGTTACC GGATAAGGCG CAGCGGTCGG GCTGAACGGG GGGTTCGTGC ACACAGCCCA GCTTGGAGCG AACGACCTAC
ACCGAACTGA GATACCTACA GCGTGAGCTA TGAGAAAGCG CCACGCTTCC CGAAGGGAGA AAGGCGGACA GGTATCCGGT
AAGCGGCAGG GTCGGAACAG GAGAGCGCAC GAGGGAGCTT CCAGGGGGAA ACGCCTGGTA TCTTTATAGT CCTGTCGGGT
TTCGCCACCT CTGACTTGAG CGTCGATTTT TGTGATGCTC GTCAGGGGGG CGGAGCCTAT GGAAAAACGC CAGCAACGCG
GCCTTTTTAC GGTTCCTGGC CTTTTGCTGG CCTTTTGCTC ACATGTTCTT TCCTGCGTTA TCCCCTGATT CTGTGGATAA
CCGTATTACC GCCTTTGAGT GAGCTGATAC CGCTCGCCGC AGCCGAACGA CCGAGCGCAG CGAGTCAGTG AGCGAGGAAG
CGGAAGAGCG CCCAATACGC AAACCGCCTC TCCCCGCGCG TTGGCCGATT CATTAATGCA GCTGGCACGA CAGGTTTCCC
GACTGGAAAG CGGGCAGTGA GCGCAACGCA ATTAATGTGA GTTAGCTCAC TCATTAGGCA CCCCAGGCTT TACACTTTAT
GCTTCCGGCT CGTATGTTGT GTGGAATTGT GAGCGGATAA CAATTTCACA CAGGAAACAG CTATGACCAT GATTACGCCA
AGCGCGCAAT TAACCCTCAC TAAAGGGAAC AAAAGCTGGA GCTGCAAGCT TAATGTAGTC TTATGCAATA CTCTTGTAGT
CTTGCAACAT GGTAACGATG AGTTAGCAAC ATGCCTTACA AGGAGAGAAA AAGCACCGTG CATGCCGATT GGTGGAAGTA
AGGTGGTACG ATCGTGCCTT ATTAGGAAGG CAACAGACGG GTCTGACATG GATTGGACGA ACCACTGAAT TGCCGCATTG
CAGAGATATT GTATTTAAGT GCCTAGCTCG ATACATAAAC GGGTCTCTCT GGTTAGACCA GATCTGAGCC TGGGAGCTCT
CTGGCTAACT AGGGAACCCA CTGCTTAAGC CTCAATAAAG CTTGCCTTGA GTGCTTCAAG TAGTGTGTGC CCGTCTGTTG
TGTGACTCTG GTAACTAGAG ATCCCTCAGA CCCTTTTAGT CAGTGTGGAA AATCTCTAGC AGTGGCGCCC GAACAGGGAC
TTGAAAGCGA AAGGGAAACC AGAGGAGCTC TCTCGACGCA GGACTCGGCT TGCTGAAGCG CGCACGGCAA GAGGCGAGGG
GCGGCGACTG GTGAGTACGC CAAAAATTTT GACTAGCGGA GGCTAGAAGG AGAGAGATGG GTGCGAGAGC GTCAGTATTA
AGCGGGGGAG AATTAGATCG CGATGGGAAA AAATTCGGTT AAGGCCAGGG GGAAAGAAAA AATATAAATT AAAACATATA
GTATGGGCAA GCAGGGAGCT AGAACGATTC GCAGTTAATC CTGGCCTGTT AGAAACATCA GAAGGCTGTA GACAAATACT
GGGACAGCTA CAACCATCCC TTCAGACAGG ATCAGAAGAA CTTAGATCAT TATATAATAC AGTAGCAACC CTCTATTGTG
TGCATCAAAG GATAGAGATA AAAGACACCA AGGAAGCTTT AGACAAGATA GAGGAAGAGC AAAACAAAAG TAAGACCACC
GCACAGCAAG CGGCCGCTGA TCTTCAGACC TGGAGGAGGA GATATGAGGG ACAATTGGAG AAGTGAATTA TATAAATATA
AAGTAGTAAA AATTGAACCA TTAGGAGTAG CACCCACCAA GGCAAAGAGA AGAGTGGTGC AGAGAGAAAA AAGAGCAGTG
GGAATAGGAG CTTTGTTCCT TGGGTTCTTG GGAGCAGCAG GAAGCACTAT GGGCGCAGCG TCAATGACGC TGACGGTACA
GGCCAGACAA TTATTGTCTG GTATAGTGCA GCAGCAGAAC AATTTGCTGA GGGCTATTGA GGCGCAACAG CATCTGTTGC
AACTCACAGT CTGGGGCATC AAGCAGCTCC AGGCAAGAAT CCTGGCTGTG GAAAGATACC TAAAGGATCA ACAGCTCCTG
GGGATTTGGG GTTGCTCTGG AAAACTCATT TGCACCACTG CTGTGCCTTG GAATGCTAGT TGGAGTAATA AATCTCTGGA
ACAGATTTGG AATCACACGA CCTGGATGGA GTGGGACAGA GAAATTAACA ATTACACAAG CTTAATACAC TCCTTAATTG
AAGAATCGCA AAACCAGCAA GAAAAGAATG AACAAGAATT ATTGGAATTA GATAAATGGG CAAGTTTGTG GAATTGGTTT
AACATAACAA ATTGGCTGTG GTATATAAAA TTATTCATAA TGATAGTAGG AGGCTTGGTA GGTTTAAGAA TAGTTTTTGC
TGTACTTTCT ATAGTGAATA GAGTTAGGCA GGGATATTCA CCATTATCGT TTCAGACCCA CCTCCCAACC CCGAGGGGAC
CCGACAGGCC CGAAGGAATA GAAGAAGAAG GTGGAGAGAG AGACAGAGAC AGATCCATTC GATTAGTGAA CGGATCTCGA
CGGTATCGAT TAGACTGTAG CCCAGGAATA TGGCAGCTAG ATTGTACACA TTTAGAAGGA AAAGTTATCT TGGTAGCAGT
TCATGTAGCC AGTGGATATA TAGAAGCAGA AGTAATTCCA GCAGAGACAG GGCAAGAAAC AGCATACTTC CTCTTAAAAT
TAGCAGGAAG ATGGCCAGTA AAAACAGTAC ATACAGACAA TGGCAGCAAT TTCACCAGTA CTACAGTTAA GGCCGCCTGT
TGGTGGGCGG GGATCAAGCA GGAATTTGGC ATTCCCTACA ATCCCCAAAG TCAAGGAGTA ATAGAATCTA TGAATAAAGA
ATTAAAGAAA ATTATAGGAC AGGTAAGAGA TCAGGCTGAA CATCTTAAGA CAGCAGTACA AATGGCAGTA TTCATCCACA
ATTTTAAAAG AAAAGGGGGG ATTGGGGGGT ACAGTGCAGG GGAAAGAATA GTAGACATAA TAGCAACAGA CATACAAACT
AAAGAATTAC AAAAACAAAT TACAAAAATT CAAAATTTTC GGGTTTATTA CAGGGACAGC AGAGATCCAG TTTGGCTGCA
TACGCGTCGT GAGGCTCCGG TGCCCGTCAG TGGGCAGAGC GCACATCGCC CACAGTCCCC GAGAAGTTGG GGGGAGGGGT
CGGCAATTGA ACCGGTGCCT AGAGAAGGTG GCGCGGGGTA AACTGGGAAA GTGATGTCGT GTACTGGCTC CGCCTTTTTC
CCGAGGGTGG GGGAGAACCG TATATAAGTG CAGTAGTCGC CGTGAACGTT CTTTTTCGCA ACGGGTTTGC CGCCAGAACA
CAGGTAAGTG CCGTGTGTGG TTCCCGCGGG CCTGGCCTCT TTACGGGTTA TGGCCCTTGC GTGCCTTGAA TTACTTCCAC
CTGGCTGCAG TACGTGATTC TTGATCCCGA GCTTCGGGTT GGAAGTGGGT GGGAGAGTTC GAGGCCTTGC GCTTAAGGAG
CCCCTTCGCC TCGTGCTTGA GTTGAGGCCT GGCCTGGGCG CTGGGGCCGC CGCGTGCGAA TCTGGTGGCA CCTTCGCGCC
TGTCTCGCTG CTTTCGATAA GTCTCTAGCC ATTTAAAATT TTTGATGACC TGCTGCGACG CTTTTTTTCT GGCAAGATAG
TCTTGTAAAT GCGGGCCAAG ATCTGCACAC TGGTATTTCG GTTTTTGGGG CCGCGGGCGG CGACGGGGCC CGTGCGTCCC
AGCGCACATG TTCGGCGAGG CGGGGCCTGC GAGCGCGGCC ACCGAGAATC GGACGGGGGT AGTCTCAAGC TGGCCGGCCT
GCTCTGGTGC CTGGCCTCGC GCCGCCGTGT ATCGCCCCGC CCTGGGCGGC AAGGCTGGCC CGGTCGGCAC CAGTTGCGTG
AGCGGAAAGA TGGCCGCTTC CCGGCCCTGC TGCAGGGAGC TCAAAATGGA GGACGCGGCG CTCGGGAGAG CGGGCGGGTG
AGTCACCCAC ACAAAGGAAA AGGGCCTTTC CGTCCTCAGC CGTCGCTTCA TGTGACTCCA CTGAGTACCG GGCGCCGTCC
AGGCACCTCG ATTAGTTCTC GTGCTTTTGG AGTACGTCGT CTTTAGGTTG GGGGGAGGGG TTTTATGCGA TGGAGTTTCC
CCACACTGAG TGGGTGGAGA CTGAAGTTAG GCCAGCTTGG CACTTGATGT AATTCTCCTT GGAATTTGCC CTTTTTGAGT
TTGGATCTTG GTTCATTCTC AAGCCTCAGA CAGTGGTTCA AAGTTTTTTT CTTCCATTTC AGGTGTCGTG AGCTAGAGCC
ACCATGGAGT TTGGGCTGAG CTGGCTTTTT CTTGTGGCTA TTTTAAAAGG TGTCCAGTGC GGATCCTCAG TTGCCAAGAA
GCATCCTAAA ACTTGGGTAC ATTACATTGC TGCTGAAGAG GAGGACTGGG ACTATGCTCC CTTAGTCCTC GCCCCCGATG
ACAGAAGTTA TAAAAGTCAA TATTTGAACA ATGGCCCTCA GCGGATTGGT AGGAAGTACA AAAAAGTCCG ATTTATGGCA
TACACAGATG AAACCTTTAA GACTCGTGAA GCTATTCAGC ATGAATCAGG AATCTTGGGA CCTTTACTTT ATGGGGAAGT
TGGAGACACA CTGTTGATTA TATTTAAGAA TCAAGCAAGC AGACCATATA ACATCTACCC TCACGGAATC ACTGATGTCC
GTCCTTTGTA TTCAAGGAGA TTACCAAAAG GTGTAAAACA TTTGAAGGAT TTTCCAATTC TGCCAGGAGA AATATTCAAA
TATAAATGGA CAGTGACTGT AGAAGATGGG CCAACTAAAT CAGATCCTCG GTGCCTGACC CGCTATTACT CTAGTTTCGT
TAATATGGAG AGAGATCTAG CTTCAGGACT CATTGGCCCT CTCCTCATCT GCTACAAAGA ATCTGTAGAT CAAAGAGGAA
ACCAGATAAT GTCAGACAAG AGGAATGTCA TCCTGTTTTC TGTATTTGAT GAGAACCGAA GCTGGTACCT CACAGAGAAT
ATACAACGCT TTCTCCCCAA TCCAGCTGGA GTGCAGCTTG AAGATCCAGA GTTCCAAGCC TCCAACATCA TGCACAGCAT
CAATGGCTAT GTTTTTGATA GTTTGCAGTT GTCAGTTTGT TTGCATGAGG TGGCATACTG GTACATTCTA AGCATTGGAG
CACAGACTGA CTTCCTTTCT GTCTTCTTCT CTGGATATAC CTTCAAACAC AAAATGGTCT ATGAAGACAC ACTCACCCTA
TTCCCATTCT CAGGAGAAAC TGTCTTCATG TCGATGGAAA ACCCAGGTCT ATGGATTCTG GGGTGCCACA ACTCAGACTT
TCGGAACAGA GGCATGACCG CCTTACTGAA GGTTTCTAGT TGTGACAAGA ACACTGGTGA TTATTACGAG GACAGTTATG
AAGATATTTC AGCATACTTG CTGAGTAAAA ACAATGCCAT TGAACCAAGA GCTAGCACCA CGACGCCAGC GCCGCGACCA
CCAACACCGG CGCCCACCAT CGCGTCGCAG CCCCTGTCCC TGCGCCCAGA GGCGTGCCGG CCAGCGGCGG GGGGCGCAGT
GCACACGAGG GGGCTGGACT TCGCCTGTGA TTCCGGAATC TACATCTGGG CCCCTCTGGC CGGCACCTGT GGCGTGCTGC
TGCTGTCCCT GGTCATCACC CTGTACTGCA AGCGGGGCAG AAAGAAGCTG CTGTACATCT TCAAGCAGCC CTTCATGCGG
CCTGTGCAGA CCACACAGGA AGAGGACGGC TGTAGCTGTA GATTCCCCGA GGAAGAGGAA GGCGGCTGCG AGCTGAGAGT
GAAGTTCAGC AGAAGCGCCG ACGCCCCTGC CTATCAGCAG GGCCAGAACC AGCTGTACAA CGAGCTGAAC CTGGGCAGAC
GGGAGGAATA CGACGTGCTG GACAAGAGAA GAGGCCGGGA CCCTGAGATG GGCGGCAAGC CCAGACGGAA GAACCCCCAG
GAAGGCCTGT ATAACGAACT GCAGAAAGAC AAGATGGCCG AGGCCTACAG CGAGATCGGC ATGAAGGGCG AGCGGAGAAG
AGGCAAGGGC CATGACGGCC TGTACCAGGG CCTGAGCACC GCCACCAAGG ACACCTACGA CGCCCTGCAC ATGCAGGCCC
TGCCTCCAAG ATGAGTCGAC AATCAACCTC TGGATTACAA AATTTGTGAA AGATTGACTG GTATTCTTAA CTATGTTGCT
CCTTTTACGC TATGTGGATA CGCTGCTTTA ATGCCTTTGT ATCATGCTAT TGCTTCCCGT ATGGCTTTCA TTTTCTCCTC
CTTGTATAAA TCCTGGTTGC TGTCTCTTTA TGAGGAGTTG TGGCCCGTTG TCAGGCAACG TGGCGTGGTG TGCACTGTGT
TTGCTGACGC AACCCCCACT GGTTGGGGCA TTGCCACCAC CTGTCAGCTC CTTTCCGGGA CTTTCGCTTT CCCCCTCCCT
ATTGCCACGG CGGAACTCAT CGCCGCCTGC CTTGCCCGCT GCTGGACAGG GGCTCGGCTG TTGGGCACTG ACAATTCCGT
GGTGTTGTCG GGGAAGCTGA CGTCCTTTCC TTGGCTGCTC GCCTGTGTTG CCACCTGGAT TCTGCGCGGG ACGTCCTTCT
GCTACGTCCC TTCGGCCCTC AATCCAGCGG ACCTTCCTTC CCGCGGCCTG CTGCCGGCTC TGCGGCCTCT TCCGCGTCTT
CGCCTTCGCC CTCAGACGAG TCGGATCTCC CTTTGGGCCG CCTCCCCGCC TGGAATTCGA GCTCGGTACC TTTAAGACCA
ATGACTTACA AGGCAGCTGT AGATCTTAGC CACTTTTTAA AAGAAAAGGG GGGACTGGAA GGGCTAATTC ACTCCCAACG
AAGACAAGAT CTGCTTTTTG CTTGTACTGG GTCTCTCTGG TTAGACCAGA TCTGAGCCTG GGAGCTCTCT GGCTAACTAG
GGAACCCACT GCTTAAGCCT CAATAAAGCT TGCCTTGAGT GCTTCAAGTA GTGTGTGCCC GTCTGTTGTG TGACTCTGGT
AACTAGAGAT CCCTCAGACC CTTTTAGTCA GTGTGGAAAA TCTCTAGCAG TAGTAGTTCA TGTCATCTTA TTATTCAGTA
TTTATAACTT GCAAAGAAAT GAATATCAGA GAGTGAGAGG AACTTGTTTA TTGCAGCTTA TAATGGTTAC AAATAAAGCA
ATAGCATCAC AAATTTCACA AATAAAGCAT TTTTTTCACT GCATTCTAGT TGTGGTTTGT CCAAACTCAT CAATGTATCT
TATCATGTCT GGCTCTAGCT ATCCCGCCCC TAACTCCGCC CAGTTCCGCC CATTCTCCGC CCCATGGCTG ACTAATTTTT
TTTATTTATG CAGAGGCCGA GGCCGCCTCG GCCTCTGAGC TATTCCAGAA GTAGTGAGGA GGCTTTTTTG GAGGCCTAGC
TAGGGACGTA CCCAATTCGC CCTATAGTGA GTCGTATTAC GCGCGCTCAC TGGCCGTCGT TTTACAACGT CGTGACTGGG
AAAACCCTGG CGTTACCCAA CTTAATCGCC TTGCAGCACA TCCCCCTTTC GCCAGCTGGC GTAATAGCGA AGAGGCCCGC
ACCGATCGCC CTTCCCAACA GTTGCGCAGC CTGAATGGCG AATGGGACGC GCCCTGTAGC GGCGCATTAA GCGCGGCGGG
TGTGGTGGTT ACGCGCAGCG TGACCGCTAC ACTTGCCAGC GCCCTAGCGC CCGCTCCTTT CGCTTTCTTC CCTTCCTTTC
TCGCCACGTT CGCCGGCTTT CCCCGTCAAG CTCTAAATCG GGGGCTCCCT TTAGGGTTCC GATTTAGTGC TTTACGGCAC
CTCGACCCCA AAAAACTTGA TTAGGGTGAT GGTTCACGTA GTGGGCCATC GCCCTGATAG ACGGTTTTTC GCCCTTTGAC
GTTGGAGTCC ACGTTCTTTA ATAGTGGACT CTTGTTCCAA ACTGGAACAA CACTCAACCC TATCTCGGTC TATTCTTTTG
ATTTATAAGG GATTTTGCCG ATTTCGGCCT ATTGGTTAAA AAATGAGCTG ATTTAACAAA AATTTAACGC GAATTTTAAC
AAAATATTAA CGCTTACAAT TTAGGTGGCA CTTTTCGGGG AAATGTGCGC GGAACCCCTA TTTGTTTATT TTTCTAAATA
CATTCAAATA TGTATCCGCT CATGAGACAA TAACCCTGAT AAATGCTTCA ATAATATTGA AAAAGGAAGA GTATGAGTAT
TCAACATTTC CGTGTCGCCC TTATTCCCTT TTTTGCGGCA TTTTGCCTTC CTGTTTTTGC TCACCCAGAA ACGCTGGTGA
AAGTAAAAGA TGCTGAAGAT CAGTTGG
pTRPE-hFVIII-C2-BBz
(SEQ ID NO: 20)
GTGCACGAGT GGGTTACATC GAACTGGATC TCAACAGCGG TAAGATCCTT GAGAGTTTTC GCCCCGAAGA ACGTTTTCCA
ATGATGAGCA CTTTTAAAGT TCTGCTATGT GGCGCGGTAT TATCCCGTAT TGACGCCGGG CAAGAGCAAC TCGGTCGCCG
CATACACTAT TCTCAGAATG ACTTGGTTGA GTACTCACCA GTCACAGAAA AGCATCTTAC GGATGGCATG ACAGTAAGAG
AATTATGCAG TGCTGCCATA ACCATGAGTG ATAACACTGC GGCCAACTTA CTTCTGACAA CGATCGGAGG ACCGAAGGAG
CTAACCGCTT TTTTGCACAA CATGGGGGAT CATGTAACTC GCCTTGATCG TTGGGAACCG GAGCTGAATG AAGCCATACC
AAACGACGAG CGTGACACCA CGATGCCTGT AGCAATGGCA ACAACGTTGC GCAAACTATT AACTGGCGAA CTACTTACTC
TAGCTTCCCG GCAACAATTA ATAGACTGGA TGGAGGCGGA TAAAGTTGCA GGACCACTTC TGCGCTCGGC CCTTCCGGCT
GGCTGGTTTA TTGCTGATAA ATCTGGAGCC GGTGAGCGTG GGTCTCGCGG TATCATTGCA GCACTGGGGC CAGATGGTAA
GCCCTCCCGT ATCGTAGTTA TCTACACGAC GGGGAGTCAG GCAACTATGG ATGAACGAAA TAGACAGATC GCTGAGATAG
GTGCCTCACT GATTAAGCAT TGGTAACTGT CAGACCAAGT TTACTCATAT ATACTTTAGA TTGATTTAAA ACTTCATTTT
TAATTTAAAA GGATCTAGGT GAAGATCCTT TTTGATAATC TCATGACCAA AATCCCTTAA CGTGAGTTTT CGTTCCACTG
AGCGTCAGAC CCCGTAGAAA AGATCAAAGG ATCTTCTTGA GATCCTTTTT TTCTGCGCGT AATCTGCTGC TTGCAAACAA
AAAAACCACC GCTACCAGCG GTGGTTTGTT TGCCGGATCA AGAGCTACCA ACTCTTTTTC CGAAGGTAAC TGGCTTCAGC
AGAGCGCAGA TACCAAATAC TGTTCTTCTA GTGTAGCCGT AGTTAGGCCA CCACTTCAAG AACTCTGTAG CACCGCCTAC
ATACCTCGCT CTGCTAATCC TGTTACCAGT GGCTGCTGCC AGTGGCGATA AGTCGTGTCT TACCGGGTTG GACTCAAGAC
GATAGTTACC GGATAAGGCG CAGCGGTCGG GCTGAACGGG GGGTTCGTGC ACACAGCCCA GCTTGGAGCG AACGACCTAC
ACCGAACTGA GATACCTACA GCGTGAGCTA TGAGAAAGCG CCACGCTTCC CGAAGGGAGA AAGGCGGACA GGTATCCGGT
AAGCGGCAGG GTCGGAACAG GAGAGCGCAC GAGGGAGCTT CCAGGGGGAA ACGCCTGGTA TCTTTATAGT CCTGTCGGGT
TTCGCCACCT CTGACTTGAG CGTCGATTTT TGTGATGCTC GTCAGGGGGG CGGAGCCTAT GGAAAAACGC CAGCAACGCG
GCCTTTTTAC GGTTCCTGGC CTTTTGCTGG CCTTTTGCTC ACATGTTCTT TCCTGCGTTA TCCCCTGATT CTGTGGATAA
CCGTATTACC GCCTTTGAGT GAGCTGATAC CGCTCGCCGC AGCCGAACGA CCGAGCGCAG CGAGTCAGTG AGCGAGGAAG
CGGAAGAGCG CCCAATACGC AAACCGCCTC TCCCCGCGCG TTGGCCGATT CATTAATGCA GCTGGCACGA CAGGTTTCCC
GACTGGAAAG CGGGCAGTGA GCGCAACGCA ATTAATGTGA GTTAGCTCAC TCATTAGGCA CCCCAGGCTT TACACTTTAT
GCTTCCGGCT CGTATGTTGT GTGGAATTGT GAGCGGATAA CAATTTCACA CAGGAAACAG CTATGACCAT GATTACGCCA
AGCGCGCAAT TAACCCTCAC TAAAGGGAAC AAAAGCTGGA GCTGCAAGCT TAATGTAGTC TTATGCAATA CTCTTGTAGT
CTTGCAACAT GGTAACGATG AGTTAGCAAC ATGCCTTACA AGGAGAGAAA AAGCACCGTG CATGCCGATT GGTGGAAGTA
AGGTGGTACG ATCGTGCCTT ATTAGGAAGG CAACAGACGG GTCTGACATG GATTGGACGA ACCACTGAAT TGCCGCATTG
CAGAGATATT GTATTTAAGT GCCTAGCTCG ATACATAAAC GGGTCTCTCT GGTTAGACCA GATCTGAGCC TGGGAGCTCT
CTGGCTAACT AGGGAACCCA CTGCTTAAGC CTCAATAAAG CTTGCCTTGA GTGCTTCAAG TAGTGTGTGC CCGTCTGTTG
TGTGACTCTG GTAACTAGAG ATCCCTCAGA CCCTTTTAGT CAGTGTGGAA AATCTCTAGC AGTGGCGCCC GAACAGGGAC
TTGAAAGCGA AAGGGAAACC AGAGGAGCTC TCTCGACGCA GGACTCGGCT TGCTGAAGCG CGCACGGCAA GAGGCGAGGG
GCGGCGACTG GTGAGTACGC CAAAAATTTT GACTAGCGGA GGCTAGAAGG AGAGAGATGG GTGCGAGAGC GTCAGTATTA
AGCGGGGGAG AATTAGATCG CGATGGGAAA AAATTCGGTT AAGGCCAGGG GGAAAGAAAA AATATAAATT AAAACATATA
GTATGGGCAA GCAGGGAGCT AGAACGATTC GCAGTTAATC CTGGCCTGTT AGAAACATCA GAAGGCTGTA GACAAATACT
GGGACAGCTA CAACCATCCC TTCAGACAGG ATCAGAAGAA CTTAGATCAT TATATAATAC AGTAGCAACC CTCTATTGTG
TGCATCAAAG GATAGAGATA AAAGACACCA AGGAAGCTTT AGACAAGATA GAGGAAGAGC AAAACAAAAG TAAGACCACC
GCACAGCAAG CGGCCGCTGA TCTTCAGACC TGGAGGAGGA GATATGAGGG ACAATTGGAG AAGTGAATTA TATAAATATA
AAGTAGTAAA AATTGAACCA TTAGGAGTAG CACCCACCAA GGCAAAGAGA AGAGTGGTGC AGAGAGAAAA AAGAGCAGTG
GGAATAGGAG CTTTGTTCCT TGGGTTCTTG GGAGCAGCAG GAAGCACTAT GGGCGCAGCG TCAATGACGC TGACGGTACA
GGCCAGACAA TTATTGTCTG GTATAGTGCA GCAGCAGAAC AATTTGCTGA GGGCTATTGA GGCGCAACAG CATCTGTTGC
AACTCACAGT CTGGGGCATC AAGCAGCTCC AGGCAAGAAT CCTGGCTGTG GAAAGATACC TAAAGGATCA ACAGCTCCTG
GGGATTTGGG GTTGCTCTGG AAAACTCATT TGCACCACTG CTGTGCCTTG GAATGCTAGT TGGAGTAATA AATCTCTGGA
ACAGATTTGG AATCACACGA CCTGGATGGA GTGGGACAGA GAAATTAACA ATTACACAAG CTTAATACAC TCCTTAATTG
AAGAATCGCA AAACCAGCAA GAAAAGAATG AACAAGAATT ATTGGAATTA GATAAATGGG CAAGTTTGTG GAATTGGTTT
AACATAACAA ATTGGCTGTG GTATATAAAA TTATTCATAA TGATAGTAGG AGGCTTGGTA GGTTTAAGAA TAGTTTTTGC
TGTACTTTCT ATAGTGAATA GAGTTAGGCA GGGATATTCA CCATTATCGT TTCAGACCCA CCTCCCAACC CCGAGGGGAC
CCGACAGGCC CGAAGGAATA GAAGAAGAAG GTGGAGAGAG AGACAGAGAC AGATCCATTC GATTAGTGAA CGGATCTCGA
CGGTATCGAT TAGACTGTAG CCCAGGAATA TGGCAGCTAG ATTGTACACA TTTAGAAGGA AAAGTTATCT TGGTAGCAGT
TCATGTAGCC AGTGGATATA TAGAAGCAGA AGTAATTCCA GCAGAGACAG GGCAAGAAAC AGCATACTTC CTCTTAAAAT
TAGCAGGAAG ATGGCCAGTA AAAACAGTAC ATACAGACAA TGGCAGCAAT TTCACCAGTA CTACAGTTAA GGCCGCCTGT
TGGTGGGCGG GGATCAAGCA GGAATTTGGC ATTCCCTACA ATCCCCAAAG TCAAGGAGTA ATAGAATCTA TGAATAAAGA
ATTAAAGAAA ATTATAGGAC AGGTAAGAGA TCAGGCTGAA CATCTTAAGA CAGCAGTACA AATGGCAGTA TTCATCCACA
ATTTTAAAAG AAAAGGGGGG ATTGGGGGGT ACAGTGCAGG GGAAAGAATA GTAGACATAA TAGCAACAGA CATACAAACT
AAAGAATTAC AAAAACAAAT TACAAAAATT CAAAATTTTC GGGTTTATTA CAGGGACAGC AGAGATCCAG TTTGGCTGCA
TACGCGTCGT GAGGCTCCGG TGCCCGTCAG TGGGCAGAGC GCACATCGCC CACAGTCCCC GAGAAGTTGG GGGGAGGGGT
CGGCAATTGA ACCGGTGCCT AGAGAAGGTG GCGCGGGGTA AACTGGGAAA GTGATGTCGT GTACTGGCTC CGCCTTTTTC
CCGAGGGTGG GGGAGAACCG TATATAAGTG CAGTAGTCGC CGTGAACGTT CTTTTTCGCA ACGGGTTTGC CGCCAGAACA
CAGGTAAGTG CCGTGTGTGG TTCCCGCGGG CCTGGCCTCT TTACGGGTTA TGGCCCTTGC GTGCCTTGAA TTACTTCCAC
CTGGCTGCAG TACGTGATTC TTGATCCCGA GCTTCGGGTT GGAAGTGGGT GGGAGAGTTC GAGGCCTTGC GCTTAAGGAG
CCCCTTCGCC TCGTGCTTGA GTTGAGGCCT GGCCTGGGCG CTGGGGCCGC CGCGTGCGAA TCTGGTGGCA CCTTCGCGCC
TGTCTCGCTG CTTTCGATAA GTCTCTAGCC ATTTAAAATT TTTGATGACC TGCTGCGACG CTTTTTTTCT GGCAAGATAG
TCTTGTAAAT GCGGGCCAAG ATCTGCACAC TGGTATTTCG GTTTTTGGGG CCGCGGGCGG CGACGGGGCC CGTGCGTCCC
AGCGCACATG TTCGGCGAGG CGGGGCCTGC GAGCGCGGCC ACCGAGAATC GGACGGGGGT AGTCTCAAGC TGGCCGGCCT
GCTCTGGTGC CTGGCCTCGC GCCGCCGTGT ATCGCCCCGC CCTGGGCGGC AAGGCTGGCC CGGTCGGCAC CAGTTGCGTG
AGCGGAAAGA TGGCCGCTTC CCGGCCCTGC TGCAGGGAGC TCAAAATGGA GGACGCGGCG CTCGGGAGAG CGGGCGGGTG
AGTCACCCAC ACAAAGGAAA AGGGCCTTTC CGTCCTCAGC CGTCGCTTCA TGTGACTCCA CTGAGTACCG GGCGCCGTCC
AGGCACCTCG ATTAGTTCTC GTGCTTTTGG AGTACGTCGT CTTTAGGTTG GGGGGAGGGG TTTTATGCGA TGGAGTTTCC
CCACACTGAG TGGGTGGAGA CTGAAGTTAG GCCAGCTTGG CACTTGATGT AATTCTCCTT GGAATTTGCC CTTTTTGAGT
TTGGATCTTG GTTCATTCTC AAGCCTCAGA CAGTGGTTCA AAGTTTTTTT CTTCCATTTC AGGTGTCGTG AGCTAGAGCC
ACCATGGAGT TTGGGCTGAG CTGGCTTTTT CTTGTGGCTA TTTTAAAAGG TGTCCAGTGC GGATCCAATA GTTGCAGCAT
GCCATTGGGA ATGGAGAGTA AAGCAATATC AGATGCACAG ATTACTGCTT CATCCTACTT TACCAATATG TTTGCCACCT
GGTCTCCTTC AAAAGCTCGA CTTCACCTCC AAGGGAGGAG TAATGCCTGG AGACCTCAGG TGAATAATCC AAAAGAGTGG
CTGCAAGTGG ACTTCCAGAA GACAATGAAA GTCACAGGAG TAACTACTCA GGGAGTAAAA TCTCTGCTTA CCAGCATGTA
TGTGAAGGAG TTCCTCATCT CCAGCAGTCA AGATGGCCAT CAGTGGACTC TCTTTTTTCA GAATGGCAAA GTAAAGGTTT
TTCAGGGAAA TCAAGACTCC TTCACACCTG TGGTGAACTC TCTAGACCCA CCGTTACTGA CTCGCTACCT TCGAATTCAC
CCCCAGAGTT GGGTGCACCA GATTGCCCTG AGGATGGAGG TTCTGGGCTG CGAGGCACAG GACCTCTACG CTAGCACCAC
GACGCCAGCG CCGCGACCAC CAACACCGGC GCCCACCATC GCGTCGCAGC CCCTGTCCCT GCGCCCAGAG GCGTGCCGGC
CAGCGGCGGG GGGCGCAGTG CACACGAGGG GGCTGGACTT CGCCTGTGAT TCCGGAATCT ACATCTGGGC CCCTCTGGCC
GGCACCTGTG GCGTGCTGCT GCTGTCCCTG GTCATCACCC TGTACTGCAA GCGGGGCAGA AAGAAGCTGC TGTACATCTT
CAAGCAGCCC TTCATGCGGC CTGTGCAGAC CACACAGGAA GAGGACGGCT GTAGCTGTAG ATTCCCCGAG GAAGAGGAAG
GCGGCTGCGA GCTGAGAGTG AAGTTCAGCA GAAGCGCCGA CGCCCCTGCC TATCAGCAGG GCCAGAACCA GCTGTACAAC
GAGCTGAACC TGGGCAGACG GGAGGAATAC GACGTGCTGG ACAAGAGAAG AGGCCGGGAC CCTGAGATGG GCGGCAAGCC
CAGACGGAAG AACCCCCAGG AAGGCCTGTA TAACGAACTG CAGAAAGACA AGATGGCCGA GGCCTACAGC GAGATCGGCA
TGAAGGGCGA GCGGAGAAGA GGCAAGGGCC ATGACGGCCT GTACCAGGGC CTGAGCACCG CCACCAAGGA CACCTACGAC
GCCCTGCACA TGCAGGCCCT GCCTCCAAGA TGAGTCGACA ATCAACCTCT GGATTACAAA ATTTGTGAAA GATTGACTGG
TATTCTTAAC TATGTTGCTC CTTTTACGCT ATGTGGATAC GCTGCTTTAA TGCCTTTGTA TCATGCTATT GCTTCCCGTA
TGGCTTTCAT TTTCTCCTCC TTGTATAAAT CCTGGTTGCT GTCTCTTTAT GAGGAGTTGT GGCCCGTTGT CAGGCAACGT
GGCGTGGTGT GCACTGTGTT TGCTGACGCA ACCCCCACTG GTTGGGGCAT TGCCACCACC TGTCAGCTCC TTTCCGGGAC
TTTCGCTTTC CCCCTCCCTA TTGCCACGGC GGAACTCATC GCCGCCTGCC TTGCCCGCTG CTGGACAGGG GCTCGGCTGT
TGGGCACTGA CAATTCCGTG GTGTTGTCGG GGAAGCTGAC GTCCTTTCCT TGGCTGCTCG CCTGTGTTGC CACCTGGATT
CTGCGCGGGA CGTCCTTCTG CTACGTCCCT TCGGCCCTCA ATCCAGCGGA CCTTCCTTCC CGCGGCCTGC TGCCGGCTCT
GCGGCCTCTT CCGCGTCTTC GCCTTCGCCC TCAGACGAGT CGGATCTCCC TTTGGGCCGC CTCCCCGCCT GGAATTCGAG
CTCGGTACCT TTAAGACCAA TGACTTACAA GGCAGCTGTA GATCTTAGCC ACTTTTTAAA AGAAAAGGGG GGACTGGAAG
GGCTAATTCA CTCCCAACGA AGACAAGATC TGCTTTTTGC TTGTACTGGG TCTCTCTGGT TAGACCAGAT CTGAGCCTGG
GAGCTCTCTG GCTAACTAGG GAACCCACTG CTTAAGCCTC AATAAAGCTT GCCTTGAGTG CTTCAAGTAG TGTGTGCCCG
TCTGTTGTGT GACTCTGGTA ACTAGAGATC CCTCAGACCC TTTTAGTCAG TGTGGAAAAT CTCTAGCAGT AGTAGTTCAT
GTCATCTTAT TATTCAGTAT TTATAACTTG CAAAGAAATG AATATCAGAG AGTGAGAGGA ACTTGTTTAT TGCAGCTTAT
AATGGTTACA AATAAAGCAA TAGCATCACA AATTTCACAA ATAAAGCATT TTTTTCACTG CATTCTAGTT GTGGTTTGTC
CAAACTCATC AATGTATCTT ATCATGTCTG GCTCTAGCTA TCCCGCCCCT AACTCCGCCC AGTTCCGCCC ATTCTCCGCC
CCATGGCTGA CTAATTTTTT TTATTTATGC AGAGGCCGAG GCCGCCTCGG CCTCTGAGCT ATTCCAGAAG TAGTGAGGAG
GCTTTTTTGG AGGCCTAGCT AGGGACGTAC CCAATTCGCC CTATAGTGAG TCGTATTACG CGCGCTCACT GGCCGTCGTT
TTACAACGTC GTGACTGGGA AAACCCTGGC GTTACCCAAC TTAATCGCCT TGCAGCACAT CCCCCTTTCG CCAGCTGGCG
TAATAGCGAA GAGGCCCGCA CCGATCGCCC TTCCCAACAG TTGCGCAGCC TGAATGGCGA ATGGGACGCG CCCTGTAGCG
GCGCATTAAG CGCGGCGGGT GTGGTGGTTA CGCGCAGCGT GACCGCTACA CTTGCCAGCG CCCTAGCGCC CGCTCCTTTC
GCTTTCTTCC CTTCCTTTCT CGCCACGTTC GCCGGCTTTC CCCGTCAAGC TCTAAATCGG GGGCTCCCTT TAGGGTTCCG
ATTTAGTGCT TTACGGCACC TCGACCCCAA AAAACTTGAT TAGGGTGATG GTTCACGTAG TGGGCCATCG CCCTGATAGA
CGGTTTTTCG CCCTTTGACG TTGGAGTCCA CGTTCTTTAA TAGTGGACTC TTGTTCCAAA CTGGAACAAC ACTCAACCCT
ATCTCGGTCT ATTCTTTTGA TTTATAAGGG ATTTTGCCGA TTTCGGCCTA TTGGTTAAAA AATGAGCTGA TTTAACAAAA
ATTTAACGCG AATTTTAACA AAATATTAAC GCTTACAATT TAGGTGGCAC TTTTCGGGGA AATGTGCGCG GAACCCCTAT
TTGTTTATTT TTCTAAATAC ATTCAAATAT GTATCCGCTC ATGAGACAAT AACCCTGATA AATGCTTCAA TAATATTGAA
AAAGGAAGAG TATGAGTATT CAACATTTCC GTGTCGCCCT TATTCCCTTT TTTGCGGCAT TTTGCCTTCC TGTTTTTGCT
CACCCAGAAA CGCTGGTGAA AGTAAAAGAT GCTGAAGATC AGTTGG
DAP12-T2A-A2-KIRS2
(SEQ ID NO: 21)
ATGGGGGGAC TTGAACCCTG CAGCAGGTTC CTGCTCCTGC CTCTCCTGCT GGCTGTAAGT GGTCTCCGTC CTGTCCAGGT
CCAGGCCCAG AGCGATTGCA GTTGCTCTAC GGTGAGCCCG GGCGTGCTGG CAGGGATCGT GATGGGAGAC CTGGTGCTGA
CAGTGCTCAT TGCCCTGGCC GTGTACTTCC TGGGCCGGCT GGTCCCTCGG GGGCGAGGGG CTGCGGAGGC AGCGACCCGG
AAACAGCGTA TCACTGAGAC CGAGTCGCCT TATCAGGAGC TCCAGGGTCA GAGGTCGGAT GTCTACAGCG ACCTCAACAC
ACAGAGGCCG TATTACAAAG TCGAGGGCGG CGGAGAGGGC AGAGGAAGTC TTCTAACATG CGGTGACGTG GAGGAGAATC
CCGGCCCTAG GATGGCCTTA CCAGTGACCG CCTTGCTCCT GCCGCTGGCC TTGCTGCTCC ACGCCGCCAG GCCGGGATCC
TCAGTTGCCA AGAAGCATCC TAAAACTTGG GTACATTACA TTGCTGCTGA AGAGGAGGAC TGGGACTATG CTCCCTTAGT
CCTCGCCCCC GATGACAGAA GTTATAAAAG TCAATATTTG AACAATGGCC CTCAGCGGAT TGGTAGGAAG TACAAAAAAG
TCCGATTTAT GGCATACACA GATGAAACCT TTAAGACTCG TGAAGCTATT CAGCATGAAT CAGGAATCTT GGGACCTTTA
CTTTATGGGG AAGTTGGAGA CACACTGTTG ATTATATTTA AGAATCAAGC AAGCAGACCA TATAACATCT ACCCTCACGG
AATCACTGAT GTCCGTCCTT TGTATTCAAG GAGATTACCA AAAGGTGTAA AACATTTGAA GGATTTTCCA ATTCTGCCAG
GAGAAATATT CAAATATAAA TGGACAGTGA CTGTAGAAGA TGGGCCAACT AAATCAGATC CTCGGTGCCT GACCCGCTAT
TACTCTAGTT TCGTTAATAT GGAGAGAGAT CTAGCTTCAG GACTCATTGG CCCTCTCCTC ATCTGCTACA AAGAATCTGT
AGATCAAAGA GGAAACCAGA TAATGTCAGA CAAGAGGAAT GTCATCCTGT TTTCTGTATT TGATGAGAAC CGAAGCTGGT
ACCTCACAGA GAATATACAA CGCTTTCTCC CCAATCCAGC TGGAGTGCAG CTTGAAGATC CAGAGTTCCA AGCCTCCAAC
ATCATGCACA GCATCAATGG CTATGTTTTT GATAGTTTGC AGTTGTCAGT TTGTTTGCAT GAGGTGGCAT ACTGGTACAT
TCTAAGCATT GGAGCACAGA CTGACTTCCT TTCTGTCTTC TTCTCTGGAT ATACCTTCAA ACACAAAATG GTCTATGAAG
ACACACTCAC CCTATTCCCA TTCTCAGGAG AAACTGTCTT CATGTCGATG GAAAACCCAG GTCTATGGAT TCTGGGGTGC
CACAACTCAG ACTTTCGGAA CAGAGGCATG ACCGCCTTAC TGAAGGTTTC TAGTTGTGAC AAGAACACTG GTGATTATTA
CGAGGACAGT TATGAAGATA TTTCAGCATA CTTGCTGAGT AAAAACAATG CCATTGAACC AAGAGCTAGC GGTGGCGGAG
GTTCTGGAGG TGGGGGTTCC TCACCCACTG AACCAAGCTC CAAAACCGGT AACCCCAGAC ACCTGCATGT TCTGATTGGG
ACCTCAGTGG TCAAAATCCC TTTCACCATC CTCCTCTTCT TTCTCCTTCA TCGCTGGTGC TCCAACAAAA AAAATGCTGC
TGTAATGGAC CAAGAGCCTG CAGGGAACAG AACAGTGAAC AGCGAGGATT CTGATGAACA AGACCATCAG GAGGTGTCAT
ACGCATAA
FVIII-A2-KIRS2
(SEQ ID NO: 22)
MALPVTALLL PLALLLHAAR PGSSVAKKHP KTWVHYIAAE EEDWDYAPLV LAPDDRSYKS QYLNNGPQRI GRKYKKVRPM
AYTDETFKTR EAIQHESGIL GPLLYGEVGD TLLIIFKNQA SRPYNIYPHG ITDVRPLYSR RLPKGVKHLK DFPILPGEIF
KYKWTVTVED GPTKSDPRCL TRYYSSFVNM ERDLASGLIG PLLICYKESV DQRGNQIMSD KRNVILFSVF DENRSWYLTE
NIQRFLPNPA GVQLEDPEFQ ASNIMHSING YVFDSLQLSV CLHEVAYWYI LSIGAQTDFL SVFFSGYTFK HKMVYEDTLT
LFPFSGETVF MSMENPGLWI LGCHNSDFRN RGMTALLKVS SCDKNTGDYY EDSYEDISAY LLSKNNAIEP RASGGGGSGG
GGSSPTEPSS KTGNPRHLHV LIGTSVVKIP FTILLFFLLH RWCSNKKNAA VMDQEPAGNR TVNSEDSDEQ DHQEVSYA*
DAP12-T2A-C2-KIRS2
(SEQ ID NO: 23)
ATGGGGGGAC TTGAACCCTG CAGCAGGTTC CTGCTCCTGC CTCTCCTGCT GGCTGTAAGT GGTCTCCGTC CTGTCCAGGT
CCAGGCCCAG AGCGATTGCA GTTGCTCTAC GGTGAGCCCG GGCGTGCTGG CAGGGATCGT GATGGGAGAC CTGGTGCTGA
CAGTGCTCAT TGCCCTGGCC GTGTACTTCC TGGGCCGGCT GGTCCCTCGG GGGCGAGGGG CTGCGGAGGC AGCGACCCGG
AAACAGCGTA TCACTGAGAC CGAGTCGCCT TATCAGGAGC TCCAGGGTCA GAGGTCGGAT GTCTACAGCG ACCTCAACAC
ACAGAGGCCG TATTACAAAG TCGAGGGCGG CGGAGAGGGC AGAGGAAGTC TTCTAACATG CGGTGACGTG GAGGAGAATC
CCGGCCCTAG GATGGCCTTA CCAGTGACCG CCTTGCTCCT GCCGCTGGCC TTGCTGCTCC ACGCCGCCAG GCCGGGATCC
AATAGTTGCA GCATGCCATT GGGAATGGAG AGTAAAGCAA TATCAGATGC ACAGATTACT GCTTCATCCT ACTTTACCAA
TATGTTTGCC ACCTGGTCTC CTTCAAAAGC TCGACTTCAC CTCCAAGGGA GGAGTAATGC CTGGAGACCT CAGGTGAATA
ATCCAAAAGA GTGGCTGCAA GTGGACTTCC AGAAGACAAT GAAAGTCACA GGAGTAACTA CTCAGGGAGT AAAATCTCTG
CTTACCAGCA TGTATGTGAA GGAGTTCCTC ATCTCCAGCA GTCAAGATGG CCATCAGTGG ACTCTCTTTT TTCAGAATGG
CAAAGTAAAG GTTTTTCAGG GAAATCAAGA CTCCTTCACA CCTGTGGTGA ACTCTCTAGA CCCACCGTTA CTGACTCGCT
ACCTTCGAAT TCACCCCCAG AGTTGGGTGC ACCAGATTGC CCTGAGGATG GAGGTTCTGG GCTGCGAGGC ACAGGACCTC
TACGCTAGCG GTGGCGGAGG TTCTGGAGGT GGGGGTTCCT CACCCACTGA ACCAAGCTCC AAAACCGGTA ACCCCAGACA
CCTGCATGTT CTGATTGGGA CCTCAGTGGT CAAAATCCCT TTCACCATCC TCCTCTTCTT TCTCCTTCAT CGCTGGTGCT
CCAACAAAAA AAATGCTGCT GTAATGGACC AAGAGCCTGC AGGGAACAGA ACAGTGAACA GCGAGGATTC TGATGAACAA
GACCATCAGG AGGTGTCATA CGCATAA
FVIII-C2-KIRS2
(SEQ ID NO: 24)
MALPVTALLL PLALLLHAAR PGSNSCSMPL GMESKAISDA QITASSYFTN MFATWSPSKA RLHLQGRSNA WRPQVNNPKE
WLQVDFQKTM KVTGVTTQGV KSLLTSMYVK EFLISSSQDG HQWTLFFQNG KVKVFQGNQD SFTPVVNSLD PPLLTRYLRI
HPQSWVHQIA LRMEVLGCEA QDLYASGGGG SGGGGSSPTE PSSKTGNPRH LHVLIGTSVV KIPFTILLFF LLHRWCSNKK
NAAVMDQEPA GNRTVNSEDS DEQDHQEVSY A*
A2-gs-BBz Nucleotide Sequence
(SEQ ID NO: 25)
ATGGAGTTTG GGCTGAGCTG GCTTTTTCTT GTGGCTATTT TAAAAGGTGT CCAGTGCGGA TCCTCAGTTG CCAAGAAGCA
TCCTAAAACT TGGGTACATT ACATTGCTGC TGAAGAGGAG GACTGGGACT ATGCTCCCTT AGTCCTCGCC CCCGATGACA
GAAGTTATAA AAGTCAATAT TTGAACAATG GCCCTCAGCG GATTGGTAGG AAGTACAAAA AAGTCCGATT TATGGCATAC
ACAGATGAAA CCTTTAAGAC TCGTGAAGCT ATTCAGCATG AATCAGGAAT CTTGGGACCT TTACTTTATG GGGAAGTTGG
AGACACACTG TTGATTATAT TTAAGAATCA AGCAAGCAGA CCATATAACA TCTACCCTCA CGGAATCACT GATGTCCGTC
CTTTGTATTC AAGGAGATTA CCAAAAGGTG TAAAACATTT GAAGGATTTT CCAATTCTGC CAGGAGAAAT ATTCAAATAT
AAATGGACAG TGACTGTAGA AGATGGGCCA ACTAAATCAG ATCCTCGGTG CCTGACCCGC TATTACTCTA GTTTCGTTAA
TATGGAGAGA GATCTAGCTT CAGGACTCAT TGGCCCTCTC CTCATCTGCT ACAAAGAATC TGTAGATCAA AGAGGAAACC
AGATAATGTC AGACAAGAGG AATGTCATCC TGTTTTCTGT ATTTGATGAG AACCGAAGCT GGTACCTCAC AGAGAATATA
CAACGCTTTC TCCCCAATCC AGCTGGAGTG CAGCTTGAAG ATCCAGAGTT CCAAGCCTCC AACATCATGC ACAGCATCAA
TGGCTATGTT TTTGATAGTT TGCAGTTGTC AGTTTGTTTG CATGAGGTGG CATACTGGTA CATTCTAAGC ATTGGAGCAC
AGACTGACTT CCTTTCTGTC TTCTTCTCTG GATATACCTT CAAACACAAA ATGGTCTATG AAGACACACT CACCCTATTC
CCATTCTCAG GAGAAACTGT CTTCATGTCG ATGGAAAACC CAGGTCTATG GATTCTGGGG TGCCACAACT CAGACTTTCG
GAACAGAGGC ATGACCGCCT TACTGAAGGT TTCTAGTTGT GACAAGAACA CTGGTGATTA TTACGAGGAC AGTTATGAAG
ATATTTCAGC ATACTTGCTG AGTAAAAACA ATGCCATTGA ACCAAGAGCT AGCGGTGGCG GAGGTTCTGG AGGTGGAGGT
TCCTCCGGAA TCTACATCTG GGCCCCTCTG GCCGGCACCT GTGGCGTGCT GCTGCTGTCC CTGGTCATCA CCCTGTACTG
CAAGCGGGGC AGAAAGAAGC TGCTGTACAT CTTCAAGCAG CCCTTCATGC GGCCTGTGCA GACCACACAG GAAGAGGACG
GCTGTAGCTG TAGATTCCCC GAGGAAGAGG AAGGCGGCTG CGAGCTGAGA GTGAAGTTCA GCAGAAGCGC CGACGCCCCT
GCCTATCAGC AGGGCCAGAA CCAGCTGTAC AACGAGCTGA ACCTGGGCAG ACGGGAGGAA TACGACGTGC TGGACAAGAG
AAGAGGCCGG GACCCTGAGA TGGGCGGCAA GCCCAGACGG AAGAACCCCC AGGAAGGCCT GTATAACGAA CTGCAGAAAG
ACAAGATGGC CGAGGCCTAC AGCGAGATCG GCATGAAGGG CGAGCGGAGA AGAGGCAAGG GCCATGACGG CCTGTACCAG
GGCCTGAGCA CCGCCACCAA GGACACCTAC GACGCCCTGC ACATGCAGGC CCTGCCTCCA AGATGA
A2-gs-BBz Amino Acid Sequence
(SEQ ID NO: 26)
MEFGLSWLFL VAILKGVQCG SSVAKKHPKT WVHYIAAEEE DWDYAPLVLA PDDRSYKSQY LNNGPQRIGR KYKKVRFMAY
TDETFKTREA IQHESGILGP LLYGEVGDTL LIIFKNQASR PYNIYPHGIT DVRPLYSRRL PKGVKHLKDF PILPGEIFKY
KWTVTVEDGP TKSDPRCLTR YYSSFVNMER DLASGLIGPL LICYKESVDQ RGNQIMSDKR NVILFSVFDE NRSWYLTENI
QRFLPNPAGV QLEDPEFQAS NIMHSINGYV FDSLQLSVCL HEVAYWYILS IGAQTDFLSV FFSGYTFKHK MVYEDTLTLF
PFSGETVFMS MENPGLWILG CHNSDFRNRG MTALLKVSSC DKNTGDYYED SYEDISAYLL SKNNAIEPRA SGGGGSGGGG
SSGIYIWAPL AGTCGVLLLS LVITLYCKRG RKKLLYIFKQ PFMRPVQTTQ EEDGCSCRFP EEEEGGCELR VKFSRSADAP
AYQQGQNQLY NELNLGRREE YDVLDKRRGR DPEMGGKPRR KNPQEGLYNE LQKDKMAEAY SEIGMKGERR RGKGHDGLYQ
GLSTATKDTY DALHMQALPP R*
C2-gs-BBz Nucleic Acid Sequence
(SEQ ID NO: 27)
ATGGAGTTTG GGCTGAGCTG GCTTTTTCTT GTGGCTATTT TAAAAGGTGT CCAGTGCGGA TCCAATAGTT GCAGCATGCC
ATTGGGAATG GAGAGTAAAG CAATATCAGA TGCACAGATT ACTGCTTCAT CCTACTTTAC CAATATGTTT GCCACCTGGT
CTCCTTCAAA AGCTCGACTT CACCTCCAAG GGAGGAGTAA TGCCTGGAGA CCTCAGGTGA ATAATCCAAA AGAGTGGCTG
CAAGTGGACT TCCAGAAGAC AATGAAAGTC ACAGGAGTAA CTACTCAGGG AGTAAAATCT CTGCTTACCA GCATGTATGT
GAAGGAGTTC CTCATCTCCA GCAGTCAAGA TGGCCATCAG TGGACTCTCT TTTTTCAGAA TGGCAAAGTA AAGGTTTTTC
AGGGAAATCA AGACTCCTTC ACACCTGTGG TGAACTCTCT AGACCCACCG TTACTGACTC GCTACCTTCG AATTCACCCC
CAGAGTTGGG TGCACCAGAT TGCCCTGAGG ATGGAGGTTC TGGGCTGCGA GGCACAGGAC CTCTACGCTA GCGGTGGCGG
AGGTTCTGGA GGTGGAGGTT CCTCCGGAAT CTACATCTGG GCCCCTCTGG CCGGCACCTG TGGCGTGCTG CTGCTGTCCC
TGGTCATCAC CCTGTACTGC AAGCGGGGCA GAAAGAAGCT GCTGTACATC TTCAAGCAGC CCTTCATGCG GCCTGTGCAG
ACCACACAGG AAGAGGACGG CTGTAGCTGT AGATTCCCCG AGGAAGAGGA AGGCGGCTGC GAGCTGAGAG TGAAGTTCAG
CAGAAGCGCC GACGCCCCTG CCTATCAGCA GGGCCAGAAC CAGCTGTACA ACGAGCTGAA CCTGGGCAGA CGGGAGGAAT
ACGACGTGCT GGACAAGAGA AGAGGCCGGG ACCCTGAGAT GGGCGGCAAG CCCAGACGGA AGAACCCCCA GGAAGGCCTG
TATAACGAAC TGCAGAAAGA CAAGATGGCC GAGGCCTACA GCGAGATCGG CATGAAGGGC GAGCGGAGAA GAGGCAAGGG
CCATGACGGC CTGTACCAGG GCCTGAGCAC CGCCACCAAG GACACCTACG ACGCCCTGCA CATGCAGGCC CTGCCTCCAA
GATGA
C2-gs-BBz Amino Acid Sequence
(SEQ ID NO: 28)
MEFGLSWLFL VAILKGVQCG SNSCSMPLGM ESKAISDAQI TASSYFTNMF ATWSPSKARL HLQGRSNAWR PQVNNPKEWL
QVDFQKTMKV TGVTTQGVKS LLTSMYVKEF LISSSQDGHQ WTLFFQNGKV KVFQGNQDSF TPVVNSLDPP LLTRYLRIHP
QSWVHQIALR MEVLGCEAQD LYASGGGGSG GGGSSGIYIW APLAGTCGVL LLSLVITLYC KRGRKKLLYI FKQPFMRPVQ
TTQEEDGCSC RFPEEEEGGC ELRVKFSRSA DAPAYQQGQN QLYNELNLGR REEYDVLDKR RGRDPEMGGK PRRKNPQEGL
YNELQKDKMA EAYSEIGMKG ERRRGKGHDG LYQGLSTATK DTYDALHMQA LPPR*

1. (canceled)

2. An isolated nucleic acid encoding a chimeric alloantigen receptor (CALLAR), wherein the isolated nucleic acid comprises nucleotide sequence encoding Factor VIII or a fragment thereof, a nucleotide sequence encoding a transmembrane domain, a nucleotide sequence encoding an intracellular domain of a costimulatory molecule, and a nucleotide sequence encoding an intracellular signaling domain.

3. (canceled)

4. The isolated nucleic acid of claim 2, wherein the Factor VIII or fragment thereof comprises an amino acid sequence selected from the group consisting of SEQ ID NO:2 and SEQ ID NO:4.

5. The isolated nucleic acid of claim 2, wherein the Factor VIII or fragment thereof is selected from the group consisting of an A2 subunit or a C2 subunit of Factor VIII.

6. The isolated nucleic acid of claim 2, wherein the nucleotide sequence encoding the transmembrane domain encodes a CD8 alpha chain hinge and transmembrane domain.

7. The isolated nucleic acid of claim 6, wherein the CD8 alpha chain hinge comprises an amino acid sequence of SEQ ID NO:7 and transmembrane domain comprises an amino acid sequence of SEQ ID NO:8.

8. The isolated nucleic acid of claim 2, wherein the nucleotide sequence encoding the intracellular domain of the costimulatory molecule comprises a nucleotide sequence encoding a 4-1BB signaling domain.

9. The isolated nucleic acid of claim 8, wherein the 4-1BB intracellular domain comprises an amino acid sequence of SEQ ID NO:10.

10. The isolated nucleic acid of claim 2, wherein the nucleotide sequence encoding the intracellular signaling domain comprises a nucleotide sequence encoding a CD3 zeta signaling domain.

11. The isolated nucleic acid of claim 10, wherein the CD3 zeta signaling domain comprises an amino acid sequence of SEQ ID NO:12.

12. A vector comprising the isolated nucleic acid of claim 2.

13. The vector of claim 12, wherein the vector is a lentiviral vector.

14. The vector of claim 12, wherein the vector is a RNA vector.

15. (canceled)

16. An isolated chimeric alloantigen receptor (CALLAR) comprising an extracellular domain comprising Factor VIII or a fragment thereof, a transmembrane domain, an intracellular domain of a costimulatory molecule, and an intracellular signaling domain.

17-25. (canceled)

26. A genetically modified cell comprising the CALLAR of claim 16.

27. The cell of claim 26, wherein:
a) the cell expresses the CALLAR and has high affinity to antibodies expressed on B cell s;
b) the cell expresses the CALLAR and induces killing of B cells expressing antibodies; and/or
c) the cell expresses the CALLAR and has limited toxicity toward healthy cells.

28-29. (canceled)

30. The cell of claim 26, wherein the cell is selected from the group consisting of a helper T cell, a cytotoxic T cell, a memory T cell, regulatory T cell, gamma delta T cell, a natural killer cell, a monocyte, a cytokine induced killer cell, a cell line thereof, and other effector cell.

31. (canceled)

32. A method for treating a disorder associated with FVIII antibodies in a subject with hemophilia, the method comprising: administering to the subject an effective amount of the genetically modified cell of claim 26, thereby treating the disorder associated with FVIII antibodies in the subject with hemophilia.

33. The method of claim 32, wherein the subject is a human.

34. The method of claim 32, wherein the modified T cell has high affinity for Factor VIII antibodies.

35-39. (canceled)

40. A genetically modified cell comprising: an isolated chimeric alloantigen receptor (CALLAR) and DAP12, wherein the CALLAR comprises an extracellular domain comprising A2 subunit of Factor VIII or C2 subunit of Factor VIII, a linker, and a fragment of a KIR, wherein the KIR comprises a transmembrane region and a cytoplasmic domain.

41. The genetically modified cell of claim 40, wherein the KIR is KIRS2 or KIR2DS2.

42-44. (canceled)

45. A method for treating a disorder associated with FVIII antibodies in a subject with hemophilia, the method comprising administering to the subject an effective amount of the genetically modified cell of claim 40, thereby treating the disorder associated with FVIII antibodies in the subject with hemophilia.
